(19) United States
(12) Patent Application Publication
(10) Pub. No.: US 2002/0151941 A1
(43) Pub. Date: Oct. 17, 2002

(54) Medical illuminator, and medical apparatus having the medical illuminator

(76) Inventors: Shinichi Okawa, Kyoto-shi (JP); Kazunari Matoba, Kyoto-shi (JP); Minoru Imazato, Kyoto-shi (JP)

Correspondence Address: KODA & ANDROLIA, Suite 3850, 2029 Century Park East, Los Angeles, CA, 90067-3024, US

(21) Appl. No.: 10/121,410

(22) Filed: Apr. 12, 2002

(30) Foreign Application Priority Data
Apr. 16, 2001 (JP) 2001-116787
Jun. 6, 2001 (JP) 2001-171285
Dec. 28, 2001 (JP) 2001-400192

(51) Int. Cl. A61F 7/00

(52) U.S. Cl. 607/99

(57) ABSTRACT

A small-sized and high-powered medical illuminating device, and other devices, such as a medical photopolymerizer and a medical hand-piece, including the medical illuminating device. The medical illuminating device includes plural light emitting components which are integrated with a base forming into a light emission module. The base includes a substrate member having at least a concave, and the light emitting component is mounted on a bottom surface of the concave. Side surfaces of the concave of the substrate member function as a reflector for reflecting the light emitted from the light emitting component towards its front.

### BACKGROUND OF THE INVENTION

1. Field of the Invention

The present invention generally relates to a medical illuminator and medical apparatuses having the medical illuminator, and particularly relates to the medical illuminator, a medical photopolymerizer (i.e. a medical light irradiator for photocuring), a medical instrument, and a medical unit, each of which is provided with the medical illuminator, in which these apparatus can be used in a dental clinic, or can be used for bleaching at home, for example.

2. Description of the Related Art

It is necessary to condense (collect or converge) light in a narrow range for illumination, or irradiation, for medical use, concretely, for a photopolymerizer, for illumination for a variety of types of instruments (for example, illumination within the oral cavity for a turbine, a motor, a scaler for dental use) or for illumination of a unit. In this respect, such an illumination differs from general illumination employed in other fields in which bright illumination is required over a broad range.

Although a halogen lamp or a xenon lamp is used for an illumination apparatus of a photopolymerizer for medical use emiting light for curing a photopolymerizing resin material (i.e. photocuring resin material) which is a dental resin, the use of a light emitting element, such as an LED (light emitting diode), or a semiconductor laser, having characteristics such that the longevity is superior to a lamp and of a lower power consumption, has been proposed.

Japanese Laid-Open Patent Publication No. 7-240536 (Gazette of Japanese Patent No. 2979522) and Japanese Laid-Open Patent Publication No. 2000-271155, disclose a photopolymerizer (i.e. a medical light irradiator for photocuring) for collecting light to be emitted from a plurality of LED elements. Also, Japanese Laid-Open Patent Publication No. 9-187825 discloses an illumination apparatus (i.e. illuminator) in which a plurality of light emitting diodes is provided within one capsule. Also, Japanese Laid-Open Patent Publication No. 2000-316881 discloses a light illuminator for directly illuminating, or irradiating, photopolymerizing resin material in which a compact light emitting element is mounted at the tip of a supporter. Also, U.S. Pat. No. 6,102,696 discloses a light illuminator for collecting light by providing a plurality of LED elements on a curved surface.

In general, a halogen lamp or a xenon lamp is used in an illumination apparatus of an instrument for medical use. In addition, in general, light is guided to the tip of an instrument by means of a light guide such as a fiber for illumination.

For example, Japanese Laid-Open Patent Publication No. 10-337292 discloses a hand piece for dental use in which a visible light LED is built into the turbine head so as to illuminate an area to be treated. However, the concrete configuration of the visible light LED is not described. Also, Japanese Laid-Open Patent Publication No. 10-137263 discloses a treatment apparatus for dental use that emits white light by providing a recess in the cathode terminal as well as an LED chip at the bottom surface and by forming a fluorescent layer on top of that.

It is necessary for the illumination of a unit for dental use to be bright and to have a natural color temperature in order to reduce, to as great a degree as possible, the creation of a silhouette by allowing the unit to be compact, light and inexpensive, and a lamp has been used conventionally for this purpose.

Other technologies in which (an) LED(s) is (are) used for the illumination, are as follows.

For example, Japanese Laid-Open Patent Publication No. 11-202164 discloses a light source module in which a great number of LEDs are arranged on a substrate, optical fibers are connected to the respective LEDs having a one-to-one relationship, and the optical fibers are bundled and drawn out. In the same Publication, the utilization of a bare chip is suggested in place of the LEDs. Also, Japanese Laid-Open Patent Publication No. 11-162232 discloses an LED illumination module in which a plurality of LED chips is mounted on a substrate in a form of a bare chip. This LED illumination module replaces a conventional fluorescent light and is used to illuminate a broad range.

On the other hand, a photopolymerizer for medical use is required to condense light in a narrow range and needs to have a high output power for shortening the illumination time, or the like. In addition, it is required to be small, light and compact in order to carry out a sensitive operation in a narrow space such as in the oral cavity. In particular, in the case that the light source itself is mounted to a portion that enters the oral cavity, or the like, the demand for miniaturization is very great. In addition, in the case that the light source itself is mounted to a portion that enters the oral cavity, or the like, it is required not to emit heat, to be able to be sterilized and to be water-resistant in addition to requirements with respect to the form. In the case in which a light emitting element is used, it is necessary to fulfill these requirements.

In the illumination provided by medical equipment it is required to condense light in a narrow range and, in particular, a photopolymerizer or an illumination apparatus mounted in a medical instrument requires a high output power. On the other hand, an illumination apparatus for sensitive operations, for example in the oral cavity, is required to be small, light and compact. In particular, in the case that the illumination apparatus itself is mounted on a part that is brought into the oral cavity, or the like, the requirement for miniaturization is very great. In addition, in the case that the illumination apparatus itself is mounted on a part that is brought into the oral cavity, or the like, it is required for the apparatus not to generate heat, to be able to be sterilized and to be water-resistant in addition to requirements relating to the form.

Though it has been proposed to use an LED or a semiconductor laser in order to meet these requirements, a plurality of such light emitting elements need to be used so as to gain the desired amount of light because the light emitting elements provided at present (for example LED elements, semiconductor laser elements) are gained by sealing LED chips or semiconductor laser chips in packages and have small outputs. However, the above described light emitting elements themselves are limited in size by the packages and, therefore, there is a limit to miniaturization of an illumination apparatus for dental use in which LEDs or semiconductor lasers are used.

In addition, as shown in the characteristics diagram of FIG. 28, even light from an LED element of which the directivity is narrow spreads to a certain degree and, therefore, the amount of light per unit area becomes further reduced.

On the other hand, light emitting elements, such as LEDs or semiconductor lasers, are provided at present in a form of devices for mounting in which bare chips in a naked form cut out of a wafer are sealed within cases or in a form of bare chips and, in general, the output per piece is small. Though an increase in the number of light emitting elements should be taken into consideration in order to gain a desired amount of light, this acts to prevent miniaturization. Therefore, the ratio of light from the light emitting elements that is practically utilized can be increased so that the miniaturization and higher output power of a photopolymerizer can be achieved while preventing an increase in the number of light emitting elements.

### SUMMARY OF THE INVENTION

Accordingly, one object of the present invention is to provide a medical illuminator (or an illumination apparatus for medical use) of which further miniaturization is possible.

Another object of the present invention is to provide a medical light irradiator for photocuring (or a photopolymerizer for medical use), a medical instrument, and a medical unit, each of which is provided with the medical illuminator that is suitably employed for medical use.

Still another object of the present invention is to provide the medical illuminator that can emit light, of which the amount per unit area is greater, by using light emitting elements.

Still another object of the present invention is to provide the medical light irradiator for photocuring in which miniaturization and higher output power can be achieved by effectively utilizing light from the light emitting elements.

In accomplishing these and other objects of the present invention, according to one aspect thereof, there is provided an illumination apparatus for medical use, or a medical illuminator, having the following configuration.

That is, the illumination apparatus for medical use illuminates by means of a light emitting element module wherein a plurality of light emitting elements are integrated.

According to the above described configuration, a compact light emitting element module of a high brightness can be used as a light source in the illumination apparatus for medical use by integrating light emitting elements.

Accordingly, it is possible to further miniaturize the illumination apparatus for medical use.

Preferably, the above described light emitting elements are bare chips or chip elements.

In the above described configuration, the bare chips cut out of a wafer are not contained in packages and are, therefore, of a small size. Accordingly, a compact light emitting element module of a high output power can be easily formed by integrating bare chips. On the other hand, even in the case where the bare chips are chip elements contained within packages, it is possible to form a compact light emitting element module with a high output power by integrating the bare chips if the power per unit area (or unit volume) is high due to the containment of a plurality of bare chips.

Preferably, the above described light emitting element module includes a condensing means in a form, or in a configuration, so as to condense light from the above described bare chips or from the above described chip elements.

In accordance with the above described configuration, the directivity can be narrowed and light can be condensed in a narrow range and, thereby, the light output from the illumination apparatus for medical use can be efficiently utilized in the case that the output light directly illuminates an area to be treated, or the like, or in the case that illumination is carried out via a light guide member.

Here, the word of “condensing” indicates “collecting light from a light source without having the purpose of image formation” and is a concept that includes all the cases wherein light is prevented from dispersing, such as a case wherein spread light is converted to parallel light.

Preferably, the above described light emitting element module is formed in a planar manner in order to secure good operability within the oral cavity and emits light from one of its major surfaces.

In the above described configuration, a plurality of light emitting elements are arranged on, for example, a substrate and, thereby, the light emitting element module is formed in a planar manner and outputs light from a major surface of which the area is comparatively large. This is favorable as a configuration for miniaturization and for enhancing the output power wherein light from light emitting elements is efficiently utilized. In addition, since the heat radiating area becomes broad, it is possible to efficiently cool the light emitting element module.

Preferably, the above described light emitting element module is covered with a transparent resin at least on the side from which the above described bare chips or the above described chip elements emit light.

According to the above described configuration, the bare chips or the chip elements can be protected by means of the resin. In addition, it is possible to condense light from the bare chips or the chip elements by forming the resin into an appropriate shape.

More preferably, the above described light emitting element module is sealed by the above described resin.

According to the above described configuration, it is possible to realize the characteristics of being able to withstand processing by an autoclave and water resistance by sealing it, and it becomes possible to be treated by a sterilization process or cleaning process by means of high temperature steam so that the light emitting element module can be repeatedly utilized.

Preferably, a condensing lens for condensing light emitted from the above described bare chips or the above described chip elements, or a parallel light conversion mechanism for converting light emitted from the above described bare chips or the above described chip elements into parallel light, is incorporated into the above described light emitting element module on the side from which the above described bare chips or the above described chip elements emit light.

According to the above described configuration, light of which the brightness is enhanced can be outputted without providing a lens, or the like, for condensing the light outside the light emitting element module, by condensing light within the light emitting element module or by restricting the light path and, thereby, the configuration of the illumination apparatus for medical use can be simplified. In addition, it is possible to efficiently condense light emitted from the bare chips or the chip elements. For example, it is possible to efficiently condense light by providing condensing lenses that correspond to individual bare chips or chip elements, respectively.

Preferably, a cooling means for cooling the above described light emitting element module is provided.

In general, a light emitting element (for example an LED) is known for the feature of not generating heat. In the light emitting element module wherein light emitting elements are integrated, however, a considerable amount of heat is generated and this cannot be ignored. According to the above described configuration, the rise in temperature in the light emitting element module can be prevented by use of the cooling means. Thereby, it becomes unnecessary to pay attention to the part with high temperature during utilization so that handling can be made easily. For example, the light emitting element module can be connected to a portion that is arranged within the oral cavity of a patient in the photopolymerizer for medical use or in an instrument for medical use.

Preferably, the above described light emitting elements are light emitting diodes or semiconductor lasers.

Though a laser, organic EL, or the like, can be utilized as a light emitting element in the illumination apparatus for medical use, a light emitting diode (LED) or a semiconductor laser is most practical.

According to another aspect of the present invention, there are provided a photopolymerizer for medical use (or a medical light irradiator for photocuring), a medical instrument and a medical unit, each of which is provided with the aforementioned illumination apparatus for medical use.

That is, the photopolymerizer for medical use is provided with the illumination apparatus for medical use having each of the above described configurations. Light from the light emitting element module such as is described above is used for illumination for curing a photopolymerizing resin material. That is to say, the light emitting elements emit light of a wavelength (for example blue light) suitable for curing the photopolymerizing resin material.

Preferably, the above described light emitting elements emit light having differing wavelengths.

According to the above described configuration, it becomes possible to cure a plurality of photopolymerizing resin materials cured by differing wavelengths through the combination of light emitting elements having differing wavelengths.

More preferably, the above described illumination apparatus for medical use includes a first light emitting element, such as is described above, that emits white light and a second light emitting element, such as is described above, that emits blue light and selectively emits the above described white light and the above described blue light.

In the above described configuration, the first and second light emitting elements may be allowed to emit light independently in order to selectively emit the above described white light and the above described blue light. In this case, the power may be separately supplied to the first light emitting element and to the second light emitting element and, for example, electrode terminals may be separately provided or the power supply may be switched by providing a switching circuit. Or, a part that includes the first light emitting element and a part that includes the second light emitting element may be exchanged so that the illuminating light can be mechanically selected in the configuration.

According to the above described configuration, white light and blue light can be used separately. For example, white light from the first light emitting element is used for illumination while blue light from the second light emitting element can be used for curing a photopolymerizing resin material.

Preferably, a light condensing mechanism, or a light collecting mechanism, is formed within the above described light emitting module.

According to the above described configuration, light from the light emitting elements is prevented from dispersing by means of the light condensing mechanism so that the light can be efficiently utilized. In addition, light that has already been condensed is emitted from the light emitting module and, therefore, the member for condensing light emitted from the light emitting module can be eliminated so that the configuration can be simplified.

The light condensing mechanism can be formed in a variety of modes as follows.

As for the first mode, the above described light emitting element module has light condensing characteristics due to its form.

For example, the light emitting element module is covered with a transparent resin and a portion of the resin through which light from the light emitting elements passes can be formed into an appropriate shape having light condensing characteristics such as a concave form or a convex form and, thereby, the dispersion of light, at least, can be prevented and light from the light emitting elements can be condensed.

As for the second mode, a light condensing lens for condensing light emitted from the above described light emitting elements or a parallel light conversion mechanism for converting light emitted from the above described light emitting elements into parallel light is incorporated into the above described light emitting element module on the side from which the above described light emitting elements emit light.

According to the above described configuration, light from the light emitting elements can be provided with an appropriate directivity.

As for the third mode, the above described light emitting elements are arranged so as to have angles so that the light emitting surfaces for emitting light respectively face a common point.

According to the above described configuration, light from the light emitting elements can be condensed to a common point.

Preferably, the above described light emitting element module is formed into a planar shape and emits light from one of its major surfaces.

The light emitting element module is formed into a planar shape by arranging the plurality of light emitting elements on, for example, a substrate so as to output light from a major surface of which the area is comparatively large. This configuration is favorable for miniaturization and for enhancing the output power by efficiently utilizing the light emitted from the light emitting elements. In addition, the heat radiating area is large and, therefore, it is possible to efficiently cool the light emitting element module.

Preferably, the above described light emitting element module is pulse driven.

According to the above described configuration, the pulse drive allows the curing rate of the photopolymerizing resin material to be easily controlled by adjusting the size, the period, or the like, of the pulse. For example, the photopolymerizing resin material is illuminated momentarily with light of a high output power and, thereby, it is possible to gain a deep polymerization depth. In addition, in the case that the photopolymerizing resin material shrinks when momentarily illuminated with a large amount of light, the amount of light is gradually increased by means of the pulse drive so that the shrinkage due to a sudden change in the amount of light can be prevented. Though, a pulse drive is not practical from the point of view of lifetime or responsiveness in the case where a lamp is used, it is possible to implement the pulse drive with the light emitting element module.

Preferably, the above described light emitting element module is arranged at a tip portion of a photopolymerizer for medical use.

According to the above described configuration, light can be illuminated from the tip portion of the photopolymerizer for medical use. At this time, light from the light emitting element module can be efficiently utilized by allowing light from the light emitting element module not to be transmitted through the photopolymerizer for medical use or by allowing the transmission distance within the photopolymerizer for medical use to be short.

Preferably, there are provided a light output part for outputting light from the above described light emitting element module to the outside in which the above described light emitting element module is arranged, and a long and narrow supporter to which this light output part is linked at one of the end portions of the supporter are provided. The direction of light that is outputted to the outside from the above described light output part is different from the longitudinal axis direction of the above described supporter.

According to the above described configuration, light is emitted from the light output part in the direction diagonal or perpendicular to the longitudinal axis direction of the supporter so that light is not emitted in the longitudinal axis direction of the supporter unlike in a conventional photopolymerizer for medical use. Accordingly, a portion that is conventionally difficult to be illuminated with light, such as a portion that is deep within a narrow space of an oral cavity, can be easily illuminated with light.

Preferably, a light output part for outputting light from the above described light emitting element module to the outside, in which the above described light emitting element module is arranged, and a long and narrow supporter to which this light output part is linked at one of the end portions of the supporter are provided. The above described supporter includes a flexible part wherein it can be bent and the bent condition can be maintained.

In the above described configuration, the angle of the light output part relative to the supporter can be appropriately set so as to emit light at an angle corresponding to the area for which the photopolymerizer for medical use is utilized. Accordingly, it is easy to use. In addition, it is not necessary to prepare a plural number of photopolymerizers for medical use having differing angles and, therefore, this is convenient.

Preferably, a cooling means for cooling the above described light emitting element module is provided.

In general, a light emitting element (for example, an LED) is characterized by not generating heat. However, when light emitting elements are integrated, the generated heat adds up to a considerable amount and this cannot be ignored. According to the above described configuration, the overheating of the light emitting element module can be prevented by means of the cooling means. Accordingly, it is not necessary pay attention to the part with the high temperature of the photopolymerizer for medical use during utilization so that handling is easy. For example, in the case that the light emitting element module is placed within the oral cavity of a patient, there is no risk of a burn, or the like.

The cooling means can be formed in a variety of modes as follows.

Preferably, the above described cooling means is a fan, a Peltier element or a heat sink.

Preferably, a light output part for outputting light from the above described light emitting element module to the outside, in which the above described light emitting element module is arranged, and a long and narrow supporter to which this light output part is linked at one of the end portions of the supporter, are provided. A path for air transmission through which air can be sent for cooling the above described light emitting element module is located in the above described supporter.

In the above described configuration, air for cooling may be sent to the light emitting element module by providing a fan within the photopolymerizer for medical use or air for cooling may be supplied from an air source provided outside of the photopolymerizer for medical use.

Preferably, a fan for cooling the above described light emitting element module is provided.

According to the above described configuration, it is not necessary to provide a supply source of air for cooling outside of the photopolymerizer for medical use and, therefore, the configuration can be made compact. In particular, in the case of a gun-type photopolymerizer for medical use, there is a sufficient space for placing a fan so that the photopolymerizer for medical use can be easily formed. It is, of course, possible to provide a fan with another type of photopolymerizer for medical use such as of a mirror-type.

Preferably, the above described light emitting element module and fan for cooling the above described light emitting element module, are placed at a tip portion of a photopolymerizer for medical use.

According to the above described configuration, light is emitted from the tip portion of the photopolymerizer for medical use and, thereby, light can be efficiently utilized within the photopolymerizer for medical use by allowing light from the light emitting element module not to be transmitted through the photopolymerizer for medical use or by allowing the transmission distance to be short. In addition, the light emitting element module can be efficiently cooled by means of the fan.

Preferably, a heat sink is attached to the above described light emitting element module.

According to the above described configuration, the heat generated by the light emitting element module can be dissipated from the heat sink.

More preferably, a fan for cooling the above described heat sink is provided. In the case that the fan is combined with the heat sink so that the heat sink provides a path for cooling air, more effective results are gained.

Preferably, the above described light emitting element module is incorporated, or built, in a metal housing.

According to the above described configuration, the heat generated by the light emitting element module can be dissipated through the metal housing. In this case, a heat sink is provided in the metal housing so that the heat can be efficiently dissipated.

Preferably, a light guide or an external lens is placed so as to be opposed to the above described light emitting element module.

According to the above described configuration, light from the light emitting element module can be led to a desired position by means of the light guide or can be condensed to a desired position by means of the external lens.

Preferably, the above described light guide is a tapered light guide.

In the above described configuration, the tapered light guide, wherein the plane of incidence from which light enters is greater than the plane of outgoing light from which light is emitted, narrows the light path from the light emitting element module. Accordingly, a narrow range can be intensively illuminated with light of a high brightness so as to increase the amount of light per unit area.

Preferably, the above described light guide or the above described external lens is removable.

According to the above described configuration, the light guide or the external lens can be removed and, therefore, it is easy to sterilize. In addition, whether approximately parallel light is emitted or condensed light is emitted, can be selected by mounting a light guide, or by mounting an external lens, to one photopolymerizer for medical use and, therefore, this is convenient.

Preferably, a plural number of light guides of the type described above, of which the forms differ from each other, can be mounted to the photopolymerizer for medical use.

According to the above described configuration, the direction in which the light is emitted or the position to which the light is emitted, can be switched by exchanging light guides and, therefore, this is convenient.

Preferably, a control part for controlling light emission of the above described light emitting elements and a power supply battery for supplying the power to the above described light emitting elements and to the above described control part, are provided within the housing.

According to the above described configuration, it is not necessary to supply the electric power from outside, or to control the photopolymerizer for medical from outside. Therefore, the photopolymerizer for medical use can be made of a cordless type.

The medical instrument is provided with the illumination apparatus for medical use having each of the above described configurations. Light from the above described light emitting element module is used for illumination within the oral cavity.

According to the above described configuration, a compact illumination apparatus for medical use of a high output power suitable for an instrument for medical use can be used.

Preferably, the above described light emitting elements are light emitting diodes that emit white light.

According to the above described configuration, white light that is favorable for illumination of the instrument for medical use can be used for illumination.

Preferably, the above described light emitting elements include a first light emitting element that emits white light and a second light emitting element that emits blue light so that the above described white light and the above described blue light can be selectively emitted.

In the above described configuration, the first and second light emitting elements may be allowed to emit light independently in order to selectively emit the above described white light and blue light. In this case, the electric power may be supplied separately to the first light emitting element and to the second light emitting element and, for example, electrode terminals may be provided separately or the electric power supply may be switched by providing a switching circuit. Or a portion that includes the first light emitting element and a portion that includes the second light emitting element, may be exchanged so that the emitted light can be mechanically selected in the configuration.

According to the above described configuration, white light and blue light can be used separately. For example, white light from the first light emitting element can be used for illumination. In addition, blue light from the second light emitting element can be used for curing a photopolymerizing resin material. Thereby, the instrument for medical use can also be used as a photopolymerizer for medical use.

Preferably, the above described light emitting element module is mounted to the head or in the vicinity thereof.

According to the above described configuration, the light emitting element module is also mounted to the head to which a tool for medical use is mounted or in the vicinity thereof and, therefore, the vicinity of the tip of the tool for medical use that is mounted to the head can be efficiently illuminated. In addition, in the case that the tool for medical use is inserted into a deep portion, the portion can be illuminated without being blocked by the surroundings.

Preferably, a light guide is provided, which leads light from the above described light emitting element module to the head or to a light projection part provided in the head or in the vicinity thereof.

According to the above described configuration, a light projection part is also provided on the head, or in the vicinity thereof, to which a tool for medical use is mounted and, therefore, the vicinity of the tip of the tool for medical use mounted to the head can be efficiently illuminated. In addition, in the case that the tool for medical use is inserted into a deep portion, the portion can be illuminated without being blocked by the surroundings. The illumination range or the directivity can be appropriately set by means of the light guide. In addition, in the case that the light emitting element module is arranged in a part at a distance away from the head, it is possible to make the head that is formed small, so as to be able to be placed within an oral cavity.

Preferably, air is utilized for cooling the above described light emitting element module.

According to the above described configuration, in an instrument for medical use that is air driven such as, for example, a turbine, the supplied air can also be utilized for cooling the light emitting element module.

The unit for medical use is provided with the illumination apparatus for medical use having each of the above described configurations. Light from the above described light emitting element module is used for illumination.

According to the above described configuration, a light source having a high brightness, of which the lifetime is long, can be used for illumination. In addition, it is possible to emit light having directivity from a simple configuration.

Preferably, the above described light emitting elements include a first light emitting element that emits white light and a second light emitting element that emits blue light so that the above described white light and the above described blue light can be selectively emitted.

In the above described configuration, the first and second light emitting elements may be allowed to emit light independently in order to selectively emit the above described white light and blue light. In this case, the power may be supplied separately to the first light emitting element and to the second light emitting element and, for example, electrode terminals may be provided separately or the power supply may be switched by providing a switching circuit. Or a portion that includes the first light emitting element and a portion that includes the second light emitting element may be exchanged so that the emitted light can be mechanically selected in the configuration.

According to the above described configuration, white light and blue light can be used separately. For example, white light from the first light emitting element is used for illumination. In addition, blue light from the second light emitting element is used for curing a photopolymerizing resin material by illuminating the entirety of an oral cavity, or by illuminating a craftwork (or an object prepared or made by a dental technician), with the blue light. Thereby, the unit for medical use can also be used as a photopolymerizer for medical use.

According to still another aspect of the present invention, there is provided the illumination apparatus for medical use having the following configuration.

That is, the illumination apparatus for medical use is provided with a beam output part and a light guide part. The plurality of light emitting elements for emitting light suitable for curing a photopolymerizing resin material is arranged in the above described beam output part. The above described light guide part leads light, from the above described beam output part, that has entered the plane of incidence to the plane of outgoing light, which is smaller than the above described plane of incidence, after the light enters the plane of incidence so as to allow the light to be emitted from the plane of outgoing light.

In the above described configuration, the light emitting elements of the beam output part are, for example, LED elements or semiconductor laser elements.

According to the above described configuration, even if a single light emitting element has a small output power, if a plurality of the light emitting elements are employed, and if a light guide part having light condensing features such as a tapered light guide, it is possible to reduce the range of light illumination in which the amount of light per unit area is greater.

According to still another aspect of the present invention, there is provided the illumination apparatus for medical use having the following configuration.

That is, the illumination apparatus for medical use is provided with a beam output part and a light guide part at its tip. The plurality of light emitting elements for emitting light suitable for curing a photo-polymerizing resin material is arranged in the above described beam output part. The above described light guide part leads light from the above described beam output part that has entered the plane of incidence to the plane of outgoing light after the light has entered the plane of incidence and allows light to be emitted from this plane of outgoing light.

In the above described configuration, the light emitting elements of the beam output part are, for example, LED elements or semiconductor laser elements.

According to the above described configuration, the plurality of light emitting elements, of which the output power is small, is collected, and approximately parallel light is emitted from the plane of outgoing light in the light guide part and, thereby, the light condensing feature can be enhanced by preventing light from the light emitting elements from spreading so that light of a high output power having a large amount of light per unit area can be emitted. In addition, the beam output part and the light guide part can be provided at the tip of the illumination apparatus for medical use that is moved closest to an area desired to be illuminated with light so that the transmission path of the light is shortened in order to reduce light transmission loss and the utilization efficiency of light can be enhanced.

Preferably, in the above described light guide part, the above described plane of outgoing light is smaller than the above described plane of incidence.

According to the above described configuration, light is condensed by means of the light guide part so that the amount of light per unit area can be further increased.

According to still another aspect of the present invention, there is provided the illumination apparatus for medical use having the following configuration.

That is, the illumination apparatus for medical use is provided at its tip with the beam output part and the narrow directivity conversion lens or with a condensing lens. The plurality of light emitting elements for emitting light suitable for curing photopolymerizing resin material is arranged in the above described beam output part. The above described conversion lens, having a narrow directivity, narrows the directivity of light from the above described beam output part. The above described condensing lens condenses light from the above described beam output part and directly emits light to the outside.

According to the above described configuration, light from the light emitting elements can be emitted after being condensed by means of the narrow directivity conversion lens or the condensing lens and, therefore, the amount light per unit area can be increased. Light can be emitted directly to the outside from the narrow directivity conversion lens or from the condensing lens so that the configuration can be simplified without using a light guide means, such as a light guide. In addition, the beam output part and the narrow directivity conversion lens or the condensing lens are provided at the tip the illumination apparatus for medical use that is moved closest to an area that is desired to be illuminated with light and, thereby, the transmission path of light is made short in order to reduce light transmission loss and the utilization efficiency of light can be enhanced.

Preferably, the narrow directivity conversion lens that is placed between the above described beam output part and the above described condensing lens, and that narrows the directivity of light from the above described respective light emitting elements, is provided.

According to the above described configuration, light from the light emitting elements enters the condensing lens after being narrowed in directivity by means of the narrow directivity conversion lens. As a result, the range into which light has been emitted from the condensing lens becomes smaller so that the amount light per unit area can be increased. It is, of course, possible to eliminate the condensing lens, and light from the light emitting elements can be directly emitted from the configuration having only the narrow directivity conversion lens.

According to still another aspect of the present invention, there is provided the illumination apparatus for medical use having the following configuration.

That is, the illumination apparatus for medical use is provided with two or more light emitting elements for emitting light suitable for curing photopolymerizing resin material and with a cooling means for cooling these light emitting elements.

For example, in the case of a light emitting element through which a large amount of current is used, the heat emission of the light emitting element cannot be ignored. According to the above described configuration, the cooling means cools the light emitting elements and, thereby, a problem due to heat emission of the light emitting element, can be prevented from occurring.

Preferably, the above described beam output part is supported at the tip portion of a long and narrow supporter. The output direction of light emitted from the above described beam output part differs from the direction in which the longitudinal axis of the above described supporter exists.

According to the above described configuration, a so-called mirror-type illumination apparatus for medical use wherein the direction of light emission is angled relative to the longitudinal axis direction of the supporter, can be formed so that, for example, a deep portion within a narrow space of the oral cavity can be easily illuminated with light.

Preferably, the above described beam output part is formed into a planar shape and outputs light from one of its major surfaces.

In the above described configuration, the beam output part is formed into a planar shape by arranging a plurality of light emitting elements on, for example, a substrate and outputs light from a major surface having a comparatively large area. This configuration is favorable for efficient utilization of emission light from the light emitting elements, for miniaturization and for enhancement of output power. In addition, since the heat dissipation area is large, it is possible to efficiently cool the light emitting elements. In addition, in the case of use within the oral cavity, when the beam output part is of a planar shape, it is easy to place the beam output part in a narrow space, such as the space between the teeth and the cheek, for utilization.

Preferably, a tip member is linked to the tip portion of the long and narrow supporter. The above described beam output part is arranged within this tip member.

According to the above described configuration, the illumination apparatus for medical use has a form, such as of a dental mirror, and the beam output part is provided in the portion corresponding to the mirror. According to the above described configuration, the illumination apparatus is easy to use because the area to be illuminated or the vicinity thereof can be easily seen.

Preferably, the above described beam output part is supported by the tip portion of the long and narrow supporter. This supporter includes a flexible part or a mechanical part wherein it can be bent and the bent condition can be maintained.

In the above described configuration, the angle of the beam output part relative to the supporter can be appropriately set so that light can be emitted at an optimal angle according to the area for which the apparatus is utilized, such as the front surface, back surface, side surface of the teeth, or the like. In addition, it is not necessary to prepare a plurality of apparatuses having different angles and, therefore, the illumination apparatus is versatile and convenient.

Preferably, the above described light guide part is removable.

According to the above described configuration, the light guide part can be removed and is easy to sterilize.

More preferably, a plurality of light guide parts of the type such as the above described light guide part having different forms can be mounted to the illumination apparatus.

According to the above described configuration, the direction in which light is emitted or the position to which the light is emitted, or the like, can be switched by exchanging light guide parts according to the symptoms or to the areas to be illuminated and, therefore, the illumination apparatus is convenient.

Preferably, the above described light emitting elements are provided, having angles wherein light is emitted toward a common point. The above described plane of incidence of the above described light guide part is placed at the above described common point.

In the above described configuration, the light emitting elements may be provided on a curved surface or may be provided on a plane by being appropriately tilted so as to have angles wherein light is emitted toward a common point. The plane of incidence of the light guide part can be made small by condensing light from the light emitting elements and, thereby, a narrower light guide part can be used.

Preferably, a cooling means for cooling the above described light emitting elements is provided.

In general, a light emitting element (for example, an LED) is characterized by not generating heat. However, when light emitting elements are integrated, the generated heat adds up to a considerable amount, and this cannot be ignored. According to the above described configuration, the overheating of the light emitting elements can be prevented by means of the cooling means. Accordingly, it is not necessary to take into account the high temperature part of the illumination apparatus for medical use during utilization so that handling is easy. For example, in the case that the beam output part of the illumination apparatus for medical use is placed within the oral cavity of a patient, there is no risk of a burn, or the like.

The cooling means can be formed in a variety of modes. For example, a fan, a Peltier element, a heat sink, or the like, can be used as the cooling means in order to dissipate heat from the light emitting elements. In addition, the housing may be formed of a material of which thermal conductivity is great, such as a metal, so that heat dissipation effects can be enhanced. In the case where a heat sink is used in the housing, heat dissipation effects can be further enhanced.

Preferably, the above described cooling means is incorporated into the above described beam output part.

According to the above described configuration, a cooling means is placed in the vicinity of the light emitting elements so that cooling can be effectively carried out and, thereby, it is easy to miniaturize the apparatus.

Preferably, the above described light emitting elements include a mixture of elements that output, or emit, light of differing wavelengths.

According to the above described configuration, there are a plural number of light emitting elements that emit light of different wavelengths for curing the respective materials of a photopolymerizing resin material gained by mixing a plurality of materials cured by differing wavelengths and, thereby, the photopolymerizing resin material can be completely cured.

Preferably, the above described light emitting elements are driven by pulse.

According to the above described configuration, the pulse drive allows the curing rate of the photopolymerizing resin material to be easily controlled by adjusting the size, the period, and the like, of the pulse. For example, the photopolymerizing resin material is illuminated momentarily with light of a high output power and, thereby, it is possible to gain a deep polymerization depth. In addition, in the case that the photopolymerizing resin material shrinks when momentarily illuminated with a large amount of light, the amount of light is gradually increased by means of the pulse drive so that the shrinkage due to a sudden change in the amount of light can be prevented. Though a pulse drive is not practical from the point of view of lifetime or responsiveness in the case when a lamp is used, it is possible to implement a pulse drive when the light emitting element is employed.

Preferably, a control part for controlling light emission of the above described light emitting elements and a power supply battery for supplying the power to the above described light emitting elements and to the above described control part, are provided within the housing.

According to the above described configuration, it is not necessary to supply the electric power from outside, or to control the electric power from outside. Therefore, the illumination apparatus for medical use can be made of a cordless type.

According to still another aspect of the present invention, there is provided a photopolymerizer for medical use having the following configuration.

The photopolymerizer for medical use is of a type that uses light emitting elements such as LEDs or semiconductor lasers and that emits light suitable for curing photopolymerizing resin material. The photopolymerizer for medical use is provided with a reflection surface for reflecting light from the above described light emitting elements.

According to the above described configuration, light from the light emitting elements can be reflected from the reflection surface so as to be directed in a desired direction and, thereby, for example, the area in front can be illuminated. The photopolymerizing resin material may be directly illuminated with light from the light emitting elements, including the reflected light, or the photopolymerizing resin material may be illuminated with light from the light emitting elements via an optical element such as a lens or a light guide. The light emitting elements may be in an arbitrary mode, such as, for example, in a mode of a device housed in a casing, in a mode of a bare chip that is in the naked condition cut out of a wafer, in a mode of a module wherein bare chips are aligned on a substrate, or in a mode of a module wherein bare chips are integrated.

Though, as for light from the light emitting elements in a conventional apparatus, only the direct light emitted toward the front, for example, is utilized while light emitted toward the sides or emitted toward the rear is not utilized, light emitted toward the sides or emitted toward the rear can be reflected from the reflection surface in a desired direction so as to be utilized, together with the direct light, for illumination of the photopolymerizing resin material according to the above described configuration.

Accordingly, light from the light emitting elements can be effectively utilized so that miniaturization and enhancement of output power can be achieved.

In addition, according to the above described configuration, a light path can be bent by reflecting, from the reflection surface, light from the light emitting elements. The reflection surface in an appropriately curved form can be used so that light from the light emitting elements can be condensed or can be converted to parallel light. Accordingly, freedom of design of the photopolymerizer is increased so that miniaturization becomes easy.

Preferably, the photopolymerizer for medical use is provided with a supporting member having two or more recesses, two or more light emitting elements placed within the above described recesses and reflection surface arranged within the above described recesses for reflecting light from the above described light emitting elements in the direction toward the openings of the above described recesses.

In the above described configuration, at least, a portion of light from a light emitting element is reflected from a reflection surface so as to be emitted from the opening of a recess. A portion of light from a light emitting element may be directly emitted from the opening of a recess without being reflected from a reflection surface. A reflection surface may be provided separately from the inner surface of a recess, or the entirety of, or a part of, the inner surface of a recess may be formed as a reflection surface. The photopolymerizing resin material may be directly illuminated with light emitted from the opening of a recess, or the photopolymerizing resin material may be illuminated with light emitted from the opening of a recess via an optical element such as a lens or a light guide. Or the light for illumination may be condensed or may be converted into parallel light. The light emitting elements are in an arbitrary mode such as, for example, in a device accommodated in a casing, in a bare chip that is in the naked condition cut out of a wafer or in a module wherein bare chips are aligned on a substrate.

In the above described configuration, light from the light emitting elements is reflected from the reflection surface within the recesses so that the light can be emitted from the openings in a desired direction.

According to the above described configuration, an increase in the amount of light for illumination of the photopolymerizing resin material can be achieved by utilizing the reflected light from the reflection surface in addition to the direct light from the light emitting elements or by maximally collecting light from the light emitting elements using the reflection surface for reflecting light in a desired direction.

Accordingly, light from the light emitting elements can be effectively utilized so that miniaturization and enhancement of output power can be achieved.

In addition, according to the above described configuration, a light path can be bent by reflecting, from the reflection surface, light from the light emitting elements. The reflecting surface in an appropriately curved form can be used so that light from the light emitting elements can be condensed or can be converted to parallel light. Accordingly, freedom of design of the photopolymerizer is increased so that miniaturization becomes easy.

Preferably, a cross sectional form of the above described reflection surface placed within the above described recesses includes a portion of an ellipse or of a parabola.

According to the above described configuration, it is easy to emit light from the light emitting elements after condensing or after conversion to parallel light.

The photopolymerizer for medical use can be formed in a variety of concrete modes as follows.

In the first mode, the above described light emitting elements are bare chips. The above described supporting member is a substrate wherein the above described recesses are created. The above described reflection surface is formed on, at least, a portion of the inner surface of the above described recesses.

According to the above described configuration, bare chips of which the volume is small are used and, therefore, the configuration for the same amount of light can be miniaturized in comparison with the case wherein a device or a module into which bare chips are incorporated is used. In addition, the recesses are created in a substrate and the reflecting surface is formed on the inner surface of the recesse and, therefore, the configuration is simplified. Furthermore, it is easy to create recesses in a substrate. Furthermore, in the case that recesses in a cup form are provided in, for example, a ceramic substrate and coating for reflection, the reflectance of the inner surface of the recesse increases so that the inner surface of the recesse can be used as the reflection surface without additionally being processed.

Preferably, an optical element is provided for condensing, or for converting into parallel light, light emitted from the above described openings of the above described recesse formed in the above described substrate.

In the above described configuration, the optical element may be placed at a position that is opposed to the openings of the substrate so as to be at a distance away from the substrate, or so as to contact the substrate. In addition, the optical element may be placed so that the entirety of, or part of, the optical element is within a recess of the substrate.

According to the above described configuration, the utilization ratio of light can be enhanced by condensing light from the light emitting elements, or by converting light from the light emitting elements into parallel light, by means of the optical element so as to prevent light from spreading.

Preferably, the above described optical element is a spherical, or aspherical, lens.

According to the above described configuration, a lens in an appropriate form wherein the two surfaces, or one surface, are (is) in a concave, or convex, form (one surface may be a plane) is used and, thereby, light from the light emitting elements can be corrected, or can be converted into parallel light. A spherical lens is inexpensive. An aspherical lens can reduce spherical aberration in comparison with a spherical lens.

Preferably, lenses of the same type as the above described lens are placed at the openings of the above described recesses created in the above described substrate and a transparent material is filled in into the insides of the above described recesses.

According to the above described configuration, the fixing of the lenses and protection of the light emitting elements can be simultaneously carried out by using a transparent material such as an epoxy resin or a silicon resin.

Preferably, the above described substrate is a ceramic substrate, an alumina substrate or a substrate wherein a metal plate is coated with an insulator.

According to the above described configuration, since the heat dissipation effect of the substrate is excellent, heat generated by the light emitting elements can be efficiently dissipated so that no problem arises due to the heat generated by the light emitting elements. In addition, the recesses can be created with high precision. In addition, it is possible to mount the substrate to a supporter for cooling.

Preferably, the above described light emitting elements are placed at a distance away from the bottoms of the above described recesses created in the above described substrate.

According to the above described configuration, an increase in the amount of light for illumination of the photopolymerizing resin material can be achieved by reflecting, from the bottom of the recesse or from the reflection surface placed above the bottom, light that is emitted from the light emitting elements and that travels toward the sides or toward the rear, that is to say, light that travels in the direction toward the bottom of the recesse so that the reflected light travels to the front.

Preferably, the above described bare chips are mounted to the above described substrate by means of wireless bonding.

According to the above described configuration, the electrodes of the bare chips and the leads of the substrate are, for example, adhered and connected. Though breaks tend to occur at the time of the autoclaving due to the difference in thermal expansion coefficients in the wire bonding wherein wires are used, the frequency of a break can be reduced in the wireless bonding wherein bare chips are directly connected to a substrate.

Preferably, the above described bare chips are an integrated wafer.

According to the above described configuration, the integrated wafer wherein bare chips are densely formed becomes a compact light source of a high brightness and, therefore, the amount of light per unit volume is large so that a highly efficient module for illumination can be formed. The integrated wafer can be regarded as a point light source and, therefore, the effects of the reflecting surfaces or the lenses become remarkable. In addition, the number of wired portions is small and manufacture is easy.

Preferably, a cross sectional form of the above described reflection surface formed on, at least, a portion of the inner surfaces of the above described recesse of the above described substrate includes a portion of an ellipse or of a parabola.

According to the above described configuration, in the case that a cross sectional form of the reflection surface includes a portion of an ellipse, light reflected from the reflection surface can be condensed to a focal point of the ellipse or to the vicinity thereof. In the case that a cross sectional form of the reflection surface includes a portion of a parabola, light reflected from the reflection surface can be converted to parallel light that is parallel to the access of the parabola. Accordingly, it is easy to emit light from the light emitting elements after condensing or after conversion to parallel light.

Preferably, a reflecting film is formed on the above described inner surfaces of the above described recesses of the above described substrate.

According to the above described configuration, in the case that the inner surfaces of the recesses after the creation of the recesses by processing a substrate are not mirror surfaces, a reflecting film having a high reflectance can be easily formed on the inner surfaces of the recesses by means of metal deposition or plating so as to form the reflection surface.

In the second mode, the above described light emitting elements are bare chips. The above described supporting member has a substrate on which the above described light emitting elements are arranged and a reflecting member. The above described reflecting plate has through holes and is placed on the above described substrate so that the inner surfaces of these through holes cover the surroundings of the above described light emitting elements arranged on the above described substrate. The above described substrate and the above described reflecting member may be connected after being formed separately or may be integrally formed at the same time. A reflecting surface, as part of the reflection surface, is formed on, at least, a portion of the above described inner surface of the above described through hole of the above described reflecting member.

According to the above described configuration, light from the light emitting elements is reflected by the reflecting surface formed on the inner surface of the through hole of the reflecting member or is emitted directly from the through holes without being reflected.

According to the above described configuration, bare chips of which the volume is small are used and, therefore, the configuration for the same amount of light can be miniaturized in comparison with the case wherein a device, or a module, into which bare chips are incorporated is used. Since the substrate and the reflecting member are formed separately, the reflecting surface can be formed without having a restriction in the process method due to the substrate. For example, even a reflecting surface in a complex form can be easily formed with a high precision. In addition, it is easy to finish as mirror surfaces, or to form reflecting films on, the inner surfaces (reflecting surface) of the through holes.

Preferably, an optical element for condensing, or conversion to parallel light, light emitted from the above described through holes of the above described reflecting member.

In the above described configuration, an optical element may be placed at a distance away from the reflecting member or on the reflecting member in a position opposed to the opening of a through hole. In addition, the optical element may be placed so that the entirety of, or part of, the optical element is within the through hole of the reflecting member.

According to the above described configuration, the utilization ratio of light can be enhanced by condensing light from the light emitting elements or converting light from the light emitting elements into parallel light by means of the optical element.

Preferably, the above described optical element is a spherical, or aspherical, lens.

According to the above described configuration, a lens in an appropriate form wherein the two surfaces or one surface are (is) in a concave, or convex, form (one surface may be a plane) is used and, thereby, light from the light emitting elements can be corrected, or can be converted into parallel light. A spherical lens is inexpensive. An aspherical lens can reduce spherical aberration in comparison with a spherical lens.

Preferably, the above described lens is arranged at the opening of the above described through hole of the above described reflecting member and a transparent material is filled in into the inside of the above described through hole.

According to the above described configuration, the fixing of the lenses and protection of the light emitting elements can be simultaneously carried out by means of a transparent material such as, for example, an epoxy resin or a silicon resin.

Preferably, the above described substrate is a ceramic substrate, an alumina substrate or a substrate wherein a metal plate is coated with an insulator.

According to the above described configuration, since the heat dissipation effect of the substrate is excellent, heat generated by the light emitting elements can be efficiently dissipated so that no problem arises due to the heat generated by the light emitting elements. In addition, it is possible to mount the substrate to a supporter for cooling.

Preferably, the above described light emitting elements are placed at a distance away from the above described substrate.

According to the above described configuration, an increase in the amount of light for illumination of the photopolymerizing resin material can be achieved by reflecting, from the bottoms of tho recesses or from the reflection surface placed above the bottom, light that is emitted from the light emitting elements and that travels toward the sides or toward the rear, that is to say, light that travels in the direction toward the bottom of the recesse so that the reflected light travels to the front.

Preferably, the above described bare chips are mounted to the above described substrate by means of wireless bonding.

According to the above described configuration, the electrodes of the bare chips and the leads of the substrate are, for example, adhered and connected. Though breaks tend to occur at the time of autoclaving due to the difference in thermal expansion coefficients in the wire bonding wherein wires are used, the frequency of breaks can be reduced in the wireless bonding wherein bare chips are directly connected to a substrate.

Preferably, the above described bare chips are an integrated wafer.

According to the above described configuration, the integrated wafer wherein bare chips are densely formed becomes a compact light source of a high brightness and, therefore, the amount of light per unit volume is large so that a highly efficient module for illumination can be formed. The integrated wafer can be regarded as a point light source and, therefore, the effects of the reflection surface or the lenses become remarkable. In addition, the number of wired portions is small and manufacture is easy.

Preferably, a cross sectional form of the above described reflecting surface formed on, at least, part of the above described inner surface of the above described through hole of the above described reflecting member includes a portion of an ellipse or of a parabola.

According to the above described configuration, it is easy to emit light from the light emitting elements as a condensing light or as a parallel light.

Preferably, a reflecting film is formed on the above described inner surfaces of the above described through holes of the above described reflecting member.

According to the above described configuration, in the case that the inner surfaces of the through holes after the creation of the through holes by processing a reflecting member are not mirror surfaces, a reflecting film having a high reflectance can be easily formed on the inner surfaces of the through holes by means of metal deposition or plating so as to form reflecting surfaces.

In the third mode, a grip part for gripping and an extension part that extends from this grip part are provided. An opening is created at the tip of this extension part or at the vicinity thereof and, then, the above described light emitting elements are arranged within a space that is connected to this opening.

According to the above described configuration, the light emitting elements are arranged close to the opening so that an outside area is directly illuminated with light emitted from the opening, and the distance (light path) between the light emitting elements and the area to be illuminated can be shortened to the minimum and, thereby, loss due to the light guide member such as a light guide can be prevented from occurring. Accordingly, the amount of light for illumination of the photopolymerizing resin material can be increased.

Here, in the above described configuration, the light emitting elements may be arranged within the recesses of the substrate as in the first mode or may be arranged on the substrate so that the surroundings of the light emitting elements are covered with the inner surfaces of the through holes of the reflecting member arranged on the substrate as in the second mode.

Preferably, condensed light or parallel light is emitted from the above described opening.

According to the above described configuration, the range of the photopolymerizing resin material that is irradiated can be prevented from spreading so that the amount of light for illumination per unit area can be increased. In addition, only the necessary range can be illuminated and, therefore, it is easy to handle this configuration.

Preferably, the above described light emitting elements are arranged so as to emit light in the direction in which the above described extension part extends. The reflection surface is provided that reflects light from the above described light emitting elements in a direction not parallel to the direction in which the above described extension part extends so as to be arranged within the above described space.

According to the above described configuration, the direction of light from the light emitting elements can be changed at the reflection surface. Accordingly, light can be emitted in a direction not parallel to the direction in which the extension part extends and, therefore, it is easy to handle the configuration. In addition, it is not necessary to provide a space in a portion on the side opposed to the light emitting elements, relative to the reflection surface, and, therefore, the tip of the extension portion can be formed of the minimum size. In addition, the thickness (denoted by the symbol “t” or example, in FIG. 76) of the part that comes into an oral cavity can be made thinner.

Preferably, a cross sectional form of the above described reflection surface arranged within the above described space includes a portion of an ellipse or of a parabola.

According to the above described configuration, in the case that a cross sectional form of the reflection surface includes a portion of an ellipse, light reflected from the reflection surface can be condensed to a focal point of the ellipse or to the vicinity thereof. In the case that a cross sectional form of the reflection surface includes a portion of a parabola, light reflected from the reflection surface can be converted to parallel light that is parallel to the access of the parabola. Accordingly, it is easy to emit light from the light emitting elements as a condensing light or a parallel light.

Preferably, the above described reflection surface arranged within the above described space is a plane and is arranged so as to form an angle of no smaller than 45 degrees and no greater than 135 degrees with respect to the direction in which the above described extension part extends or with respect to the side of the above described grip part.

According to the above described configuration, light is emitted from the recesses in a direction approximately −90° to +90° relative to the direction that the extension part extends and relative to the side of the grip part. That is to say, light is emitted from the opening at the tip of the extension part, or in the vicinity thereof, in the direction perpendicular to the direction in which the extension part extends or in the direction tilted to the user's side, that is to say, to the grip part side. Accordingly, it becomes easy to illuminate the photopolymerizing resin material with light.

In the fourth mode, a light guide is provided so that the above described light emitting elements are arranged so as to be opposed to the end surface of incidence of this light guide.

According to the above described configuration, light from the light emitting elements arranged inside of a photopolymerizer for medical use is allowed to enter the light guide so that the photopolymerizing resin material can be illuminated with light that is emitted from the end surface of outgoing light of the light guide. Light from the light emitting elements is allowed to efficiently enter the light guide and, thereby, the amount of light for illumination of the photopolymerizing resin material can be increased.

Here, in the above described configuration, the light emitting elements may be arranged within the recesses of the substrate in the same manner as in the first mode or may be arranged on the substrate so that the surroundings thereof are covered with the inner surfaces of the through holes in the reflecting member arranged on the substrate in the same manner as in the second mode.

Preferably, the condensed light or parallel light is emitted from the above described light emitting elements so as to illuminate the above described end surface of incidence of the above described light guide.

According to the above described configuration, light from the light emitting elements is allowed to efficiently enter the light guide and, thereby, the amount of light for illumination of the photopolymerizing resin material can be increased.

In addition, the heat generated by the light emitting elements cannot be ignored because it becomes necessary to arrange the light emitting elements in a sealed small space in a photopolymerizer for medical use. Moreover, in the case that the integration density of the light emitting elements is increased or light emitting elements of a large output power are used in order to increase the amount of the light, the amount of heat generation increases.

Preferably, a cooling means for cooling the light emitting elements is further provided.

According to the above described configuration, the light emitting elements are cooled by the cooling means and, thereby, a problem due to the heat generation of the light emitting elements can be prevented from occurring. Thereby, it becomes unnecessary to pay attention to the part becoming hot during utilization of a photopolymerizer for medical use, so that handling can be made easier. Therefore, with the constitution, for example, the light emitting element module can be provided in a portion thereof that is arranged within the oral cavity of a patient.

### BRIEF DESCRIPTION OF THE DRAWING

These and other objects and features of the present invention will become clear from the following description taken in conjunction with the preferred embodiments thereof with reference to the accompanying drawings.

FIG. 1 is a perspective view of an illumination apparatus for medical use, or a medical illuminator, according to a first embodiment of the present invention.

FIG. 2 is a configuration view (or construction view) of a mirror-type photopolymerizer for medical use, or a medical light irradiator for photocuring of a mirror-type, according to an embodiment of the present invention, in which the photopolymerizer has the illumination apparatus for medical use.

FIG. 3 is a configuration view (or construction view) of a gun-type photopolymerizer for medical use, or a medical light irradiator for photocuring of a gun-type, according to an embodiment of the present invention, in which the photopolymerizer has the illumination apparatus for medical use.

FIG. 4 is a perspective view of an illumination apparatus for medical use according to a second embodiment of the present invention.

FIG. 5 is a front view of the apparatus of FIG. 4.

FIG. 6 is a partially broken side view of the apparatus of FIG. 4.

FIG. 7 is a perspective view of an illumination apparatus for medical use according to a third embodiment of the present invention.

FIG. 8 is a front view of the apparatus of FIG. 7.

FIG. 9 is a partially broken side view of the apparatus of FIG. 7.

FIG. 10 is a configuration view of a mirror-type photopolymerizer for medical use provided with a cooling fin, according to an embodiment of the present invention, in which the photopolymerizer has the illumination apparatus for medical use.

FIG. 11 is a configuration view of a mirror-type photopolymerizer for medical use provided with a cooling fan, according to an embodiment of the present invention, in which the photopolymerizer has the illumination apparatus for medical use.

FIG. 12 is a perspective view of an illumination apparatus for medical use according to a third embodiment of the present invention.

FIG. 13 is a front view of the apparatus of FIG. 12.

FIG. 14 is a side view of the apparatus of FIG. 12.

FIG. 15 is a configuration view of a hand piece, as a medical instrument, according to an embodiment of the present invention, in which the hand piece has the illumination apparatus for medical use.

FIG. 16 is a configuration view of a hand piece according to a modification to that of FIG. 15.

FIG. 17 is an enlarged view of a portion of the hand piece of FIG. 16.

FIG. 18 is a configuration view of a mirror-type photopolymerizer for medical use provided with a flexible part, according to an embodiment of the present invention, in which the photopolymerizer has the illumination apparatus for medical use.

FIG. 19 is a configuration view of a unit for medical use, or a medical unit, according to an embodiment of the present invention, in which the medical unit has the illumination apparatus for medical use.

FIG. 20 is a configuration view of a light of FIG. 19.

FIG. 21 is a configuration view of a light according to a modification to that of FIG. 20.

FIG. 22 is a configuration view of a mirror-type photopolymerizer for medical use, or a medical light irradiator for photocuring of a mirror-type, according to a modification to the embodiment of the present invention, in which the photopolymerizer has the illumination apparatus for medical use.

FIG. 23 is a configuration view of a mirror-type photopolymerizer for medical use, or a medical light irradiator for photocuring of a mirror-type, according to a modification to the embodiment of the present invention, in which the photopolymerizer has the illumination apparatus for medical use.

FIG. 24 is a perspective view of an illumination apparatus for medical use, according to a modification to the embodiment.

FIG. 25 is a front view of the apparatus of FIG. 24.

FIG. 26 is a partially broken side view of the apparatus of FIG. 24.

FIG. 27 is a side view of an illumination apparatus for medical use, according to a modification to the embodiment.

FIG. 28 is a direction characteristic diagram of a light emitting element.

FIG. 29 is a front view of an illumination apparatus for medical use according to a fifth embodiment of the present invention.

FIG. 30 is a side view of the apparatus of FIG. 29.

FIG. 31 is a front view of an illumination apparatus for medical use according to a modification to that of the embodiment.

FIG. 32 is a side view of FIG. 31.

FIG. 33 is a cross-sectional side view of an illumination apparatus for medical use according to a sixth embodiment of the present invention.

FIG. 34 is a cross-sectional side view of an illumination apparatus for medical use according to a modification to that of the embodiment.

FIG. 35 is a cross-sectional side view of an illumination apparatus for medical use according to a modification to that of the embodiment.

FIG. 36 is a configuration view (or construction view) of a gun-type photopolymerizer for medical use, or a medical light irradiator for photocuring of a gun-type, according to an embodiment of the present invention, in which the photopolymerizer has the illumination apparatus for medical use.

FIG. 37 is a configuration view (or construction view) of a mirror-type photopolymerizer for medical use, or a medical light irradiator for photocuring of a mirror-type, according to an embodiment of the present invention, in which the photopolymerizer has the illumination apparatus for medical use.

FIG. 38 is a perspective view of an illumination apparatus for medical use, according to a modification to the embodiment.

FIG. 39 is a front view of FIG. 38.

FIG. 40 is a partially broken side view of FIG. 38.

FIG. 41 is a perspective view of an illumination apparatus for medical use, according to a modification to the embodiment.

FIG. 42 is a front view of FIG. 41.

FIG. 43 is a partially broken side view of FIG. 41.

FIG. 44 is a configuration view (or construction view) of a mirror-type photopolymerizer for medical use, or a medical light irradiator for photocuring of a mirror-type, according to an embodiment of the present invention, in which the photopolymerizer has the illumination apparatus for medical use.

FIG. 45 is a side view of an illumination apparatus for medical use, according to a modification to the embodiment.

FIG. 46 is a plan view of an illumination apparatus for medical use according to a seventh embodiment of the present invention.

FIG. 47 is a cross sectional side view of a portion of the illumination apparatus for medical use of FIG. 46.

FIG. 48 is a plan view of an illumination apparatus for medical use according to a eighth embodiment of the present invention.

FIG. 49 is a cross sectional side view of a portion of the illumination apparatus for medical use of FIG. 48.

FIG. 50 is a plan view of an illumination apparatus for medical use according to a ninth embodiment of the present invention.

FIG. 51 is a cross sectional side view of a portion of the illumination apparatus for medical use of FIG. 50.

FIG. 52 is a plan view of an illumination apparatus for medical use according to a tenth embodiment of the present invention.

FIG. 53 is a side view of FIG. 52.

FIG. 54 is a cross sectional side view of a portion of the illumination apparatus for medical use of FIG. 52.

FIG. 55 is a plan view of an illumination apparatus for medical use according to an eleventh embodiment of the present invention.

FIG. 56 is a cross-sectional side view of FIG. 55.

FIG. 57 is an enlarged cross-sectional side view of a portion of FIG. 56.

FIG. 58 is a plan view of an illumination apparatus for medical use according to a twelfth embodiment of the present invention.

FIG. 59 is a cross sectional side view of the illumination apparatus for medical use of FIG. 58.

FIG. 60 is a plan view of an illumination apparatus for medical use according to a thirteenth embodiment of the present invention.

FIG. 61 is a cross-sectional side view of the illumination apparatus for medical use of FIG. 60.

FIG. 62 is a plan view of an illumination apparatus for medical use according to a fourteenth embodiment of the present invention.

FIG. 63 is a cross-sectional side view of the illumination apparatus for medical use of FIG. 62.

FIG. 64 is a plan view of an illumination apparatus for medical use according to a fifteenth embodiment of the present invention.

FIG. 65 is an enlarged cross-sectional side view of a part of the illumination apparatus for medical use of FIG. 64.

FIG. 66 is a perspective view of an illumination apparatus for medical use according to a sixteenth embodiment of the present invention.

FIG. 67 is an enlarged cross-sectional side view of a part of the illumination apparatus for medical use of FIG. 67.

FIG. 68 is a perspective view of an illumination apparatus for medical use according to a seventeenth embodiment of the present invention.

FIG. 69 is an enlarged cross-sectional side view of a part of the illumination apparatus for medical use of FIG. 68.

FIG. 70 is a perspective view of an illumination apparatus for medical use according to an eighteenth embodiment of the present invention.

FIG. 71 is an enlarged cross-sectional side view of a part of the illumination apparatus for medical use of FIG. 70.

FIG. 72 is a perspective view of an illumination apparatus for medical use according to a nineteenth embodiment of the present invention.

FIG. 73 is an enlarged cross-sectional side view of a part of the illumination apparatus for medical use of FIG. 72.

FIG. 74 is a perspective view of an illumination apparatus for medical use according to a twentieth embodiment of the present invention.

FIG. 75 is an enlarged cross-sectional side view of a part of the illumination apparatus for medical use of FIG. 74.

FIG. 76 is a configuration view, as a cross-sectional side view, of a main part of a photopolymerizer for medical use, according to an embodiment of the present invention, in which the photopolymerizer has the illumination apparatus for medical use.

FIG. 77 is a configuration view, as a cross-sectional side view, of a main part of a photopolymerizer for medical use, according to an embodiment of the present invention, in which the photopolymerizer has the illumination apparatus for medical use.

FIG. 78 is a configuration view, as a cross-sectional side view, of a main part of a photopolymerizer for medical use, according to an embodiment of the present invention, in which the photopolymerizer has the illumination apparatus for medical use.

FIG. 79 is a configuration view, as a cross-sectional side view, of a main part of a photopolymerizer for medical use, according to an embodiment of the present invention, in which the photopolymerizer has the illumination apparatus for medical use.

FIG. 80 is a configuration view, as a cross-sectional side view, of a main part of a photopolymerizer for medical use, according to an embodiment of the present invention, in which the photopolymerizer has the illumination apparatus for medical use.

FIG. 81 is a configuration view, as a cross-sectional side view, of a main part of a photopolymerizer for medical use, according to an embodiment of the present invention, in which the photopolymerizer has the illumination apparatus for medical use.

FIG. 82 is a configuration view, as a cross-sectional side view, of a gun-type photopolymerizer for medical use, according to an embodiment of the present invention, in which the photopolymerizer has the illumination apparatus for medical use.

FIG. 83 is an enlarged cross-sectional view of a main part of the photopolymerizer of FIG. 82.

FIG. 84 is a configuration view, as a cross-sectional side view, of a gun-type photopolymerizer for medical use, according to an embodiment of the present invention, in which the photopolymerizer has the illumination apparatus for medical use.

FIG. 85 is an enlarged cross-sectional view of a main part of the photopolymerizer of FIG. 84.

FIG. 86 is a side configuration view of a cordless gun type photopolymerizer for medical use, according to an embodiment of the present invention, in which an electric cord or wire is not connected thereto.

FIG. 87 is a side configuration view of a dental mirror type photopolymerizer for medical use, according to an embodiment of the present invention.

FIG. 88 is a side configuration view of a dental mirror type photopolymerizer for medical use, according to an embodiment of the present invention.

FIG. 89 is a side configuration view of a dental mirror type photopolymerizer for medical use, according to an embodiment of the present invention.

FIG. 90 is a side configuration view of a dental mirror type photopolymerizer for medical use, according to an embodiment of the present invention.

FIG. 91 is a side configuration view of a dental mirror type photopolymerizer for medical use, according to an embodiment of the present invention.

FIG. 92 is a side configuration view of a dental mirror type photopolymerizer for medical use, according to an embodiment of the present invention.

FIG. 93 is an explanatory view of the progress of light emitted from a light emitting element.

FIG. 94 is an explanatory view of the progress of light emitted from a light emitting element.

FIG. 95 is a plan view of a wafer for an integrated circuit.

FIG. 96 is a side view of the wafer of FIG. 95.

### DETAILED DESCRIPTION OF THE PREFERRED EMBODIMENT

Before a description of preferred embodiments of the present invention proceeds, it is to be noted that like or corresponding parts or components are designated by like reference numerals throughout the accompanying drawings.

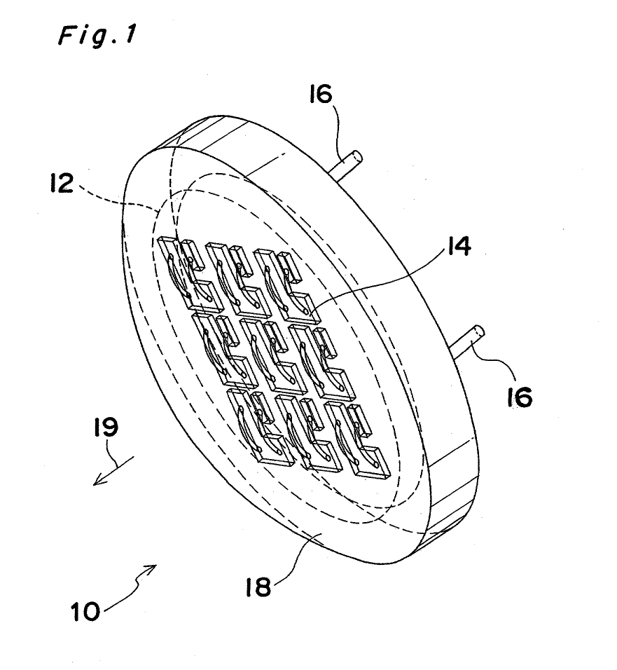

With reference to FIGS. 1 through 96, the description is made below upon an illumination apparatus for medical use (or a medical illuminator), according to each of 1st through 20th embodiments of the present invention, upon a photopolymerizer for medical use (or a medical light irradiator for photocuring) to which the illumination apparatus for medical use applies, upon an instrument for medical use (or a medical instrument) to which the illumination apparatus for medical use applies, and upon a unit for medical use (or a medical unit) to which the illumination apparatus for medical use applies.

First, with reference to FIG. 1, it is explained about an illumination apparatus for medical use according to the first embodiment of the present invention.

That is, FIG. 1 is a perspective view showing a configuration of a light emitting module 10 as an illumination apparatus for medical use. A substrate 12, on which a plurality of bare chips 14 are arranged, is covered with a resin mold 18 in the light emitting element module 10, which emits light in the direction shown by an arrow 19.

The bare chip 14 is cut out of a wafer and is a unit element forming a light emitting diode. Elements, such as resisters, are arranged on the substrate 12 so as to form a wired film integrated circuit in the same manner as a substrate of a hybrid IC (hybrid integrated circuit) into which a semiconductor circuit is incorporated. The bare chip 14 is incorporated onto the substrate 12 by means of wire bonding, or the like.

It is possible for such a configuration to be formed only of the bare chips 14 and the substrate 12 in order to reduce the amount of heat generation so that the elements such as resisters are not mounted onto the substrate 12 but inside a control circuit. It is also effective to utilize ceramic as a material for the substrate 12 in order to reduce the generation of heat.

The resin mold 18 is made of a transparent resin and covers the substrate 12 in which the bare chips 14 are built or incorporated. The light emitting elements 14 are sealed inside the resin mold 18, so that the inner components like the bare chips 14 are protected when the light emitting module 10 is cleaned and/or sterilized. It is preferable that the light emitting module 10 can be sterilized by autoclave.

The resin mold 18 is formed in a planar form along the substrate 12, and a portion on the side opposed to a bare chip 14 may be in a form (for example a form such as a convex lens or a concave lens) that is appropriate for condensing or dispersing light emitted from the bare chip 14. In a case where it is used for a photopolymerizer for medical use, for example, it is preferable for the light to be condensed so as to have a diameter of approximately 10 mm at a position 10 mm away from the emission surface.

Electrode pins 16 for the supply of the power source are provided on the side opposed to the bare chips 14 so that a voltage is applied to each of the base chips 14 via the substrate 12 in order to allow the bare chips 14 to emit light. This embodiment shows a construction in which two electrode pins 16 are mounted. However, the construction is not limited to this particular one. For example, the light emitting module 10 may have four electrode pins as described below. In addition, contacts in a spherical form may be provided in the configuration so that the electric power is supplied via the contacts.

A great number of bare chips 14 are incorporated in the light emitting module 10. Therefore, it is compact and has a high brightness in comparison with a general LED element containing one bare chip within a single package.

A plural number of types of bare chips, of which the characteristics differ from each other, may be arranged on the substrate 12, instead of arranging the bare chips 14 of the identical characteristics. For example, a plural number of bare chips which emit light of different wavelengths are incorporated into one light emitting module. In this case, control to select the wavelength of the emission light becomes easy when electrode pins are provided for the respective wavelength of the emission light.

In addition, the bare chips 14 may be placed on the substrate 12 having different angles so as to emit light toward the common point. In this case, light from the bare chips 14 can be condensed on the common point.

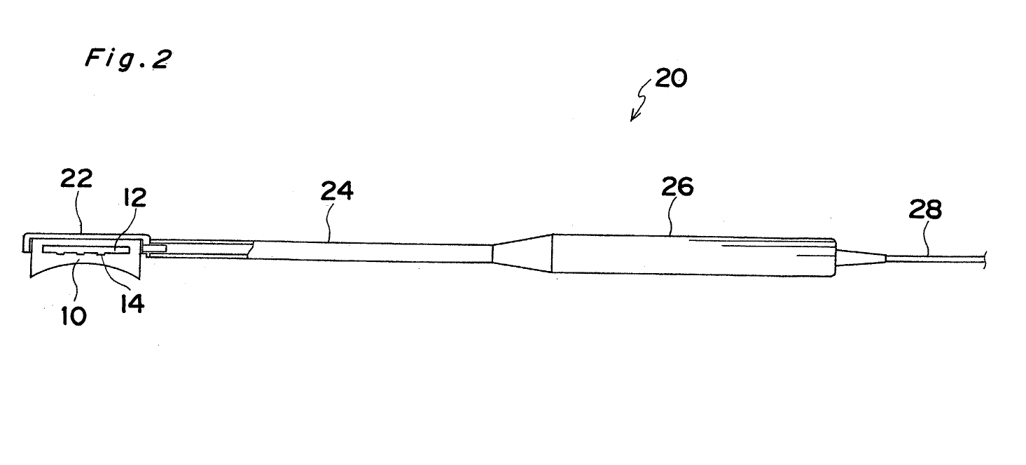

FIG. 2 shows an embodiment of a mirror-type photopolymerizer 20 for medical use provided with the light emitting module 10. The photopolymerizer 20 is, in particular, preferable for dental use.

The substrate 12 side of the light emitting module 10 is attached to a light output part 22 so as to emit light to the outside from the bare chips 14 side. The light output part 22 is supported by one end of a supporter 24 in a narrow and long axis form while the other end of the supporter 24 is fixed to a grip part 26 which can be held by hand. A power supply code 28 for supplying the electric power to the light emitting module 10 is connected to the grip part 26. Light is emitted from the light emitting module 10 in a direction different from the longitudinal direction of the supporter 24, for example, in the direction perpendicular to the longitudinal direction of the supporter 24.

Bare chips emitting light of different wavelengths may be incorporated into the light emitting module 10. A light emitting element module that emits white light and blue light can, for example, be used so that only the white light is turned on for the usage of illumination and only the blue light is turned on for photopolymerization. In addition, a light emitting element module that emits white light of which the wavelengths slightly differ can be used so that photopolymerizing resin materials (for example, dental resins) having different characteristics can be used.

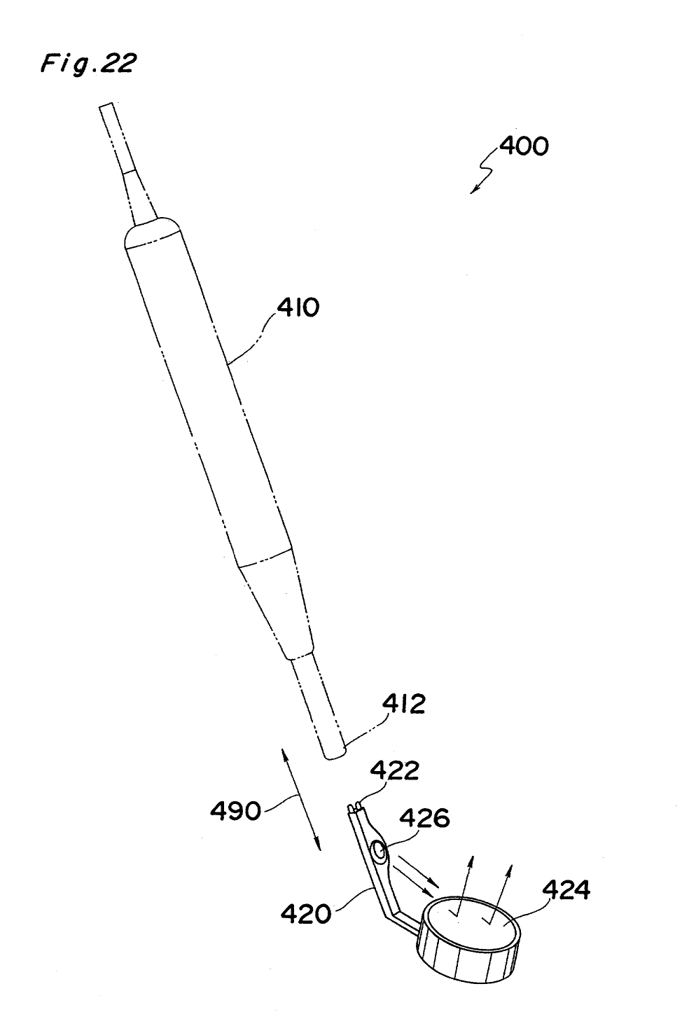

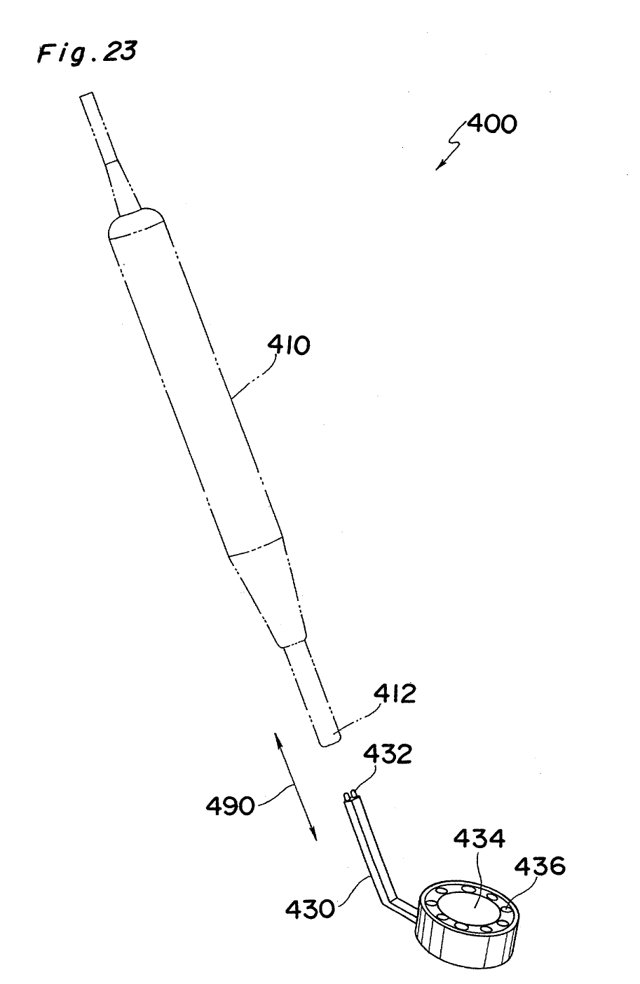

FIGS. 22 and 23 show other embodiments of mirror-type photopolymerizers 400 for medical use. As shown in the figures, white light or blue light may be mechanically selected by exchanging tip parts 420 and 430 that are removable from the body 410 in a manner indicated by an arrow 490.

More specifically, FIG. 22 shows a case where the photopolymerizer 400 for medical use is used as a mirror with a light. When the tip part 420 is connected to the body 410, connectors 412 and 422 are electrically connected so that light emitting diode 426 provided in the tip part 420 emits light. The light emitting diode 426 emits white light toward a mirror 424.

On the other hand, FIG. 23 shows a case where the photopolymerizer 400 for medical use is used as a photopolymerizer. When a tip part 430 is connected to the body 410, connectors 412 and 432 are electrically connected so that light emitting diodes 436 provided in the tip part 430 emit light. The light emitting diodes 436 are arranged around a mirror 434 and emit blue light that is suitable for curing a photopolymerizing resin material.

The output of the light emitting module 10 may be constant or may be varied. The output may, for example, be increased step by step. Or the amount of light may be gradually increased by gradually increasing the duty. In addition, a pulse drive for instant emissions of light may be carried out. The pulse drive can easily control the curing rate of a photopolymerizing resin material by adjusting the size, the period, or the like, of the pulse. In the case that, for example, a photopolymerizing resin material is instantly illuminated with output light of a high power, it is possible to gain a deep photopolymerization. Though the pulse drive is not practical from the point of view of longevity or from the point of view of response in the case that a lamp is used, it is possible to employ the pulse drive in case that a light emitting element module is used.

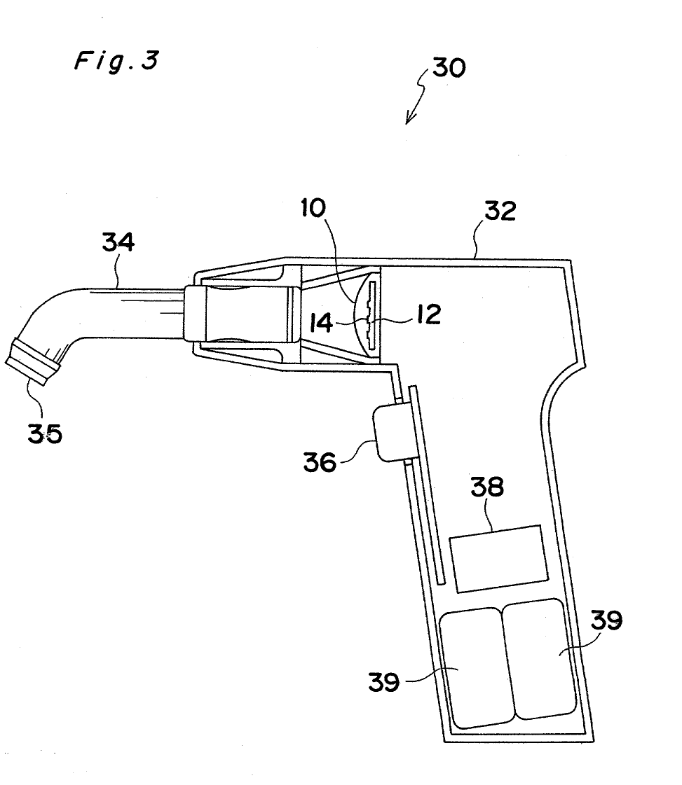

FIG. 3 shows an embodiment of a gun-type photopolymerizer 30 for medical use provided with the light emitting module 10. The photopolymerizer 30 is, in particular, favorable for dental use.

A light guide 34 is attached to an end portion of a housing 32 formed in an approximately L-shape in the photopolymerizer 30 for medical use. The light emitting module 10 is arranged within the housing 32 so as to be opposed to one end surface of the light guide 34 while light is emitted from the other end surface 35 of the light guide 34. A control circuit substrate 38 and power supply batteries 39 are accommodated within the housing 32 so that the bare chip 14 in the light emitting module 10 emits light when an operational switch 36 protruding from the housing 32 is pressed.

The light guide 34, which have a high possibility of coming into contact with the teeth, can be removed from the photopolymerizer 30 for medical use so as to be sterilized. In addition, a variety of light guides 34 of which the forms differ can be prepared, and a particular guide 34 can be selected and mounted on the housing 32 in accordance with the purpose of utilization. In the case that, for example, a tapered-type light guide wherein a large number of optical fibers in a tapered form are bundled in the same direction, is mounted thereon, a narrow range can be illuminated in a concentrated manner with light of a high brightness. In the case of a tapered-type light guide of which the incident surface has a diameter of 15 mm, wherein the emission surface has a diameter of 8 mm and of which the length is 10 mm, the amount of light per unit volume can be expected to become approximately three times greater.

A lens may be provided between the light emitting module 10 and the light guide 34 in order to enhance the characteristics of light condensing or in order to efficiently utilize light. In this case, the lens may be made removable so as to be exchangeable with a lens of an appropriate characteristic of light condensing compatible with the mounted light guide 34.

Here, in the same manner as in the case of the above described gun-type photopolymerizer for medical use, a mirror-type photopolymerizer for medical use as shown in FIG. 2 can be configured in which a light guide can be mounted or in which a lens can be provided. In this case, the light guide having a high possibility of coming into contact with the teeth may be removable so as to be sterilized in the same manner as in the above. In addition, when the tapered-type light guide is mounted, a narrow range can be illuminated in a concentrated manner with light of a high brightness.

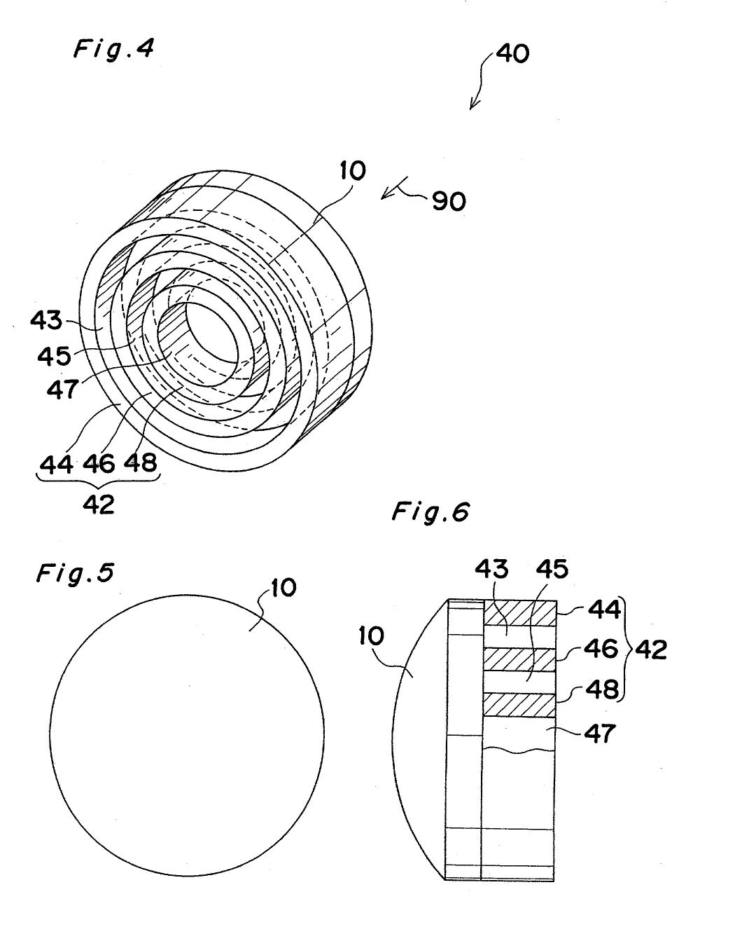

Next, with reference to FIGS. 4 to 6, it is explained about an illumination apparatus for medical use (or a medical illuminator) 40, according to the second embodiment of the present invention.

FIG. 4 is a perspective view seen from the substrate side of the light emitting module 10; FIG. 5 is a front view thereof seen from a direction (i.e. from a bare chip side) shown by an arrow 90 in FIG. 4; and FIG. 6 is a side view thereof. A heatsink 42 is attached to the substrate side of the light emitting module 10 so that the heat generated in the light emitting module 10 is dissipated therefrom. The heatsink 42 has a plurality of fins 44, 46 and 48 in a cylindrical concentric form that will not cause damage to the oral cavity, and has a plurality of spaces 43, 45 and 47 so as to increase the area of heat radiation.

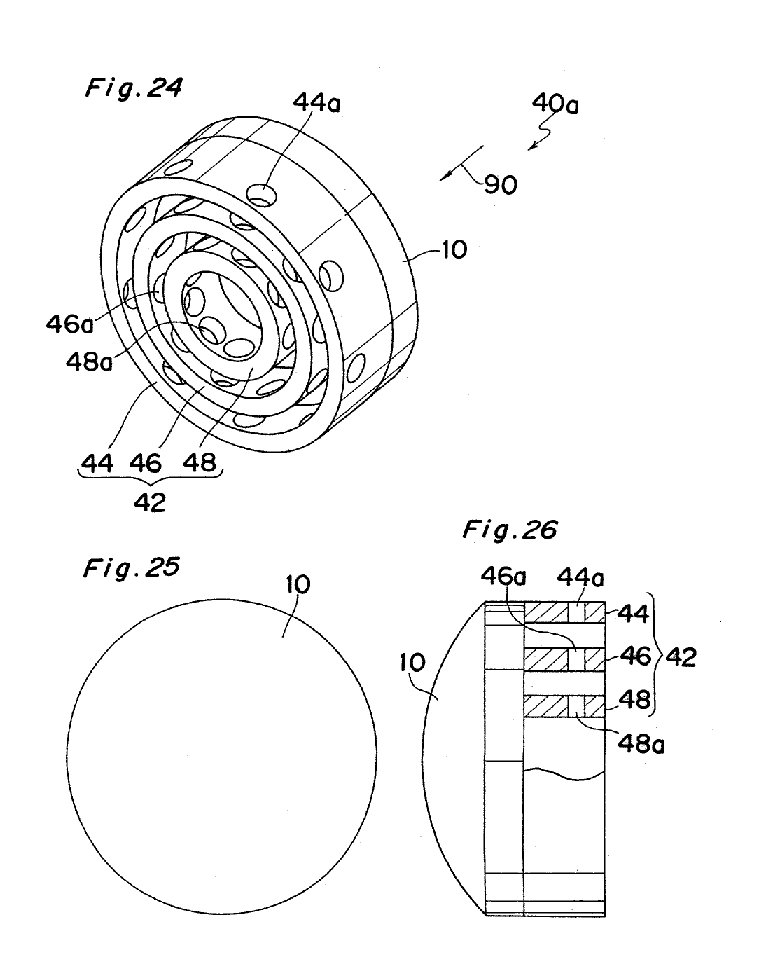

As the illumination apparatus for medical use, as a modification, shown in FIG. 24, in order to further enhance the cooling effect, penetrating holes 44a, 46a and 48a can be provided respectively in the fins 44, 46 and 48 of the heatsink 42 attached to the light emitting module 10, and air can be blown through the heat sink 42 by a fan so as to let air pass therein.

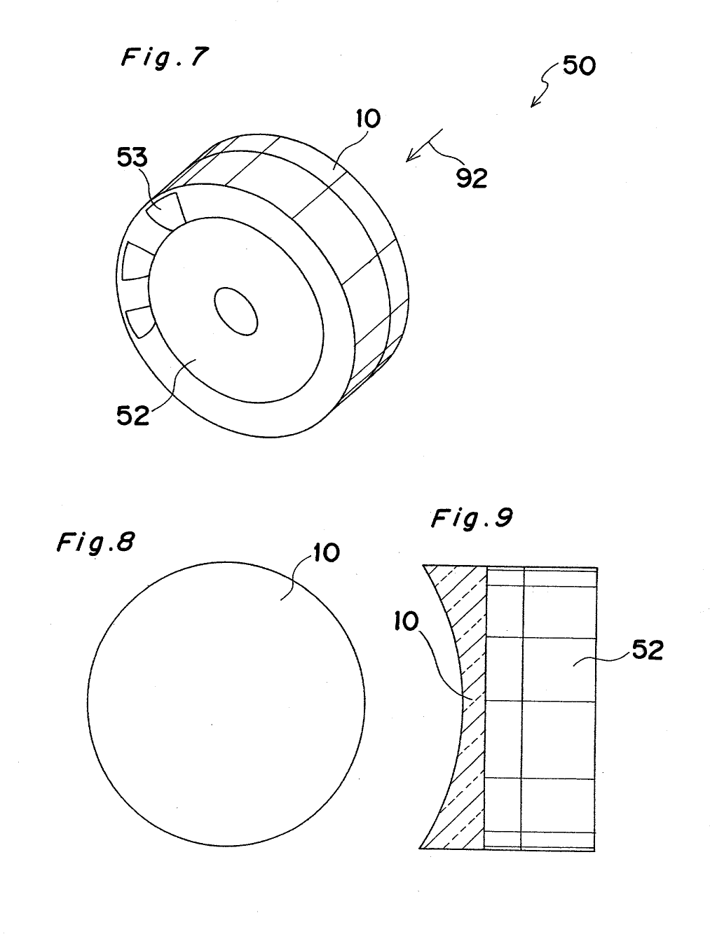

Next, with reference to FIGS. 7 through 9, it is explained about an illumination apparatus for medical use 50 according to the third embodiment of the present invention.

FIG. 7 is a perspective view of a light emitting module 10 seen from the substrate side; FIG. 8 is a front view thereof seen from a direction shown by an arrow 92 in FIG. 7; and FIG. 9 is a partially broken side view of FIG. 7. A fan 52 is attached to the substrate side of the light emitting module 10 in the illumination apparatus 50. The fan 52 blows air to the light emitting module 10 for cooling it by means of a plurality of rotating blades 53 (only three blades are illustrated in the figure, and the remaining blades are omitted therein).

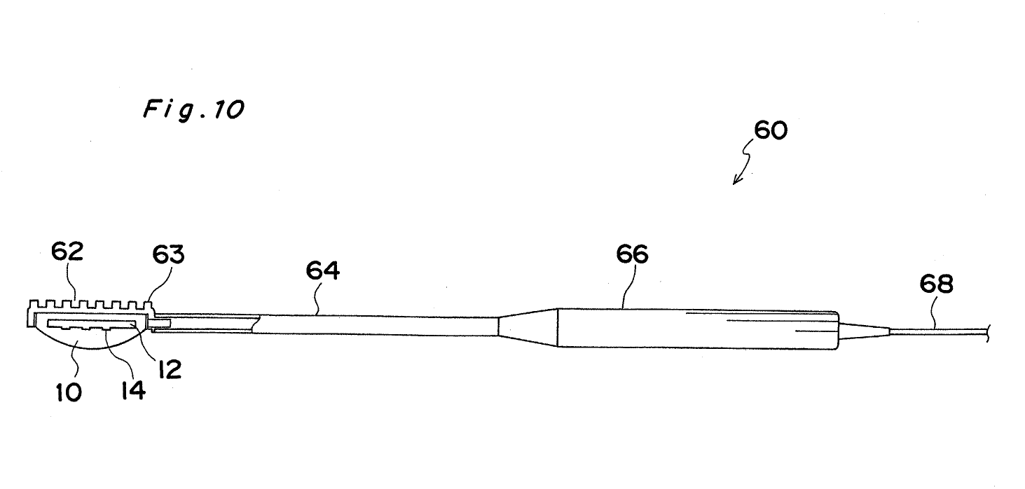

FIG. 10 shows an embodiment of a mirror-type photopolymerizer 60 for medical use provided with the light emitting module 10, in which a plurality of cooling fins 63 are provided on its light output part 62. The light output part 62 has a metal member so that the heat from the light emitting module 10 is efficiently conveyed to the cooling fins 63. The cooling fins 63 are formed in the cylindrical concentric form like the heat sink 42 in FIG. 4. A supporter 64, a grip part 66 and a power supply cord 68 are formed in the same manner as in photopolymerizer 20 for medical use in FIG. 2.

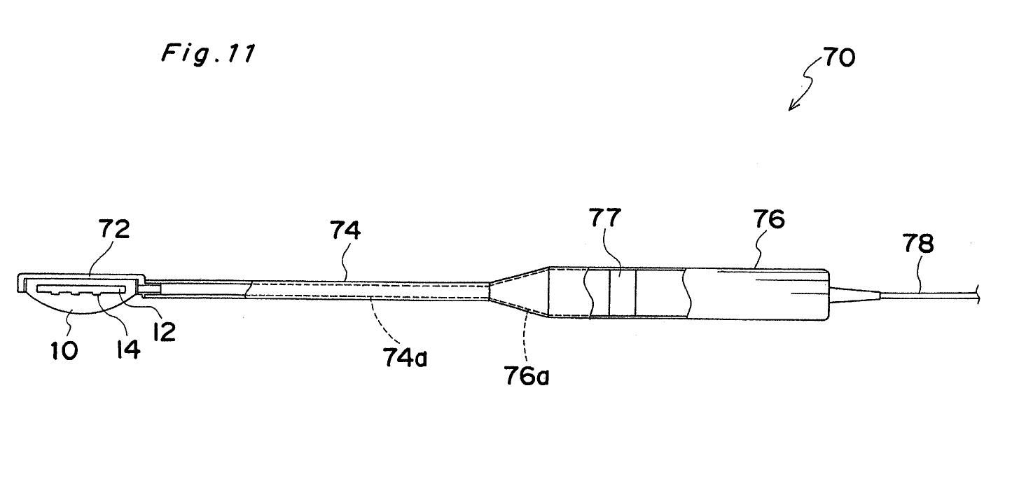

FIG. 11 shows an embodiment of a mirror-type photopolymerizer 60 for medical use provided with the light emitting module 10, in which the light emitting module 10 is cooled by providing a fan 77 within the grip part 76 thereof. The supporter 74 and the grip part 76 are formed hollow in which paths of blown air 74a and 76a are provided. In the construction, air for cooling the module 10 fed from the fan 77 is fed to the light emitting module 10 attached to its light output part 72. The electric power is supplied to the fan 77 from an electric power supply cord 78.

Next, with reference to FIGS. 12 through 14, it is explained about an illumination apparatus for medical use 50 according to the fourth embodiment of the present invention.

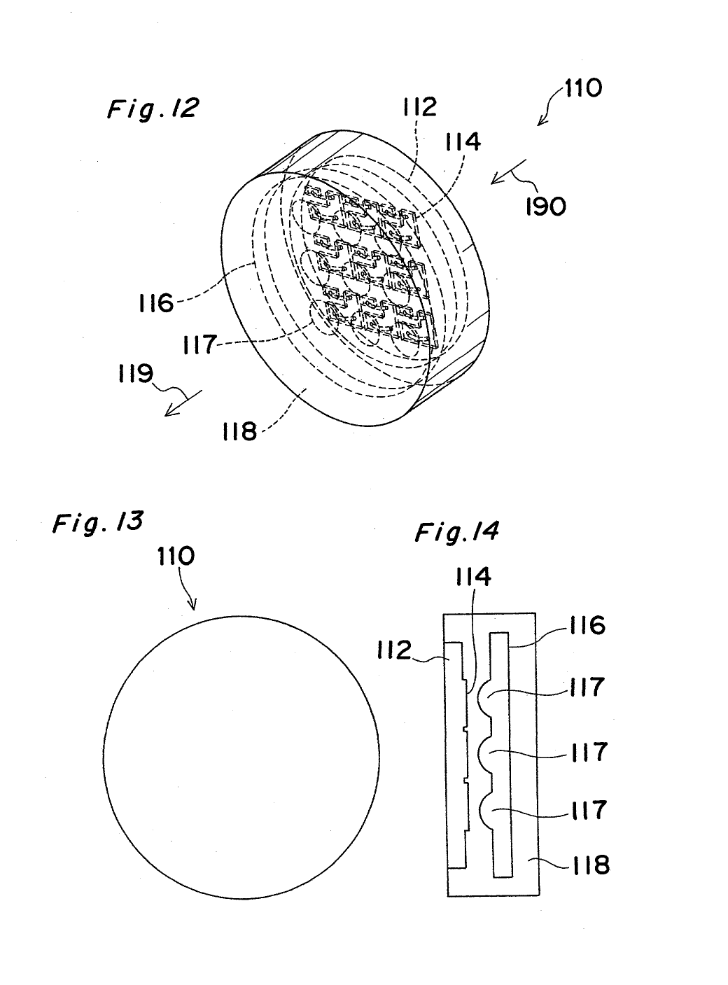

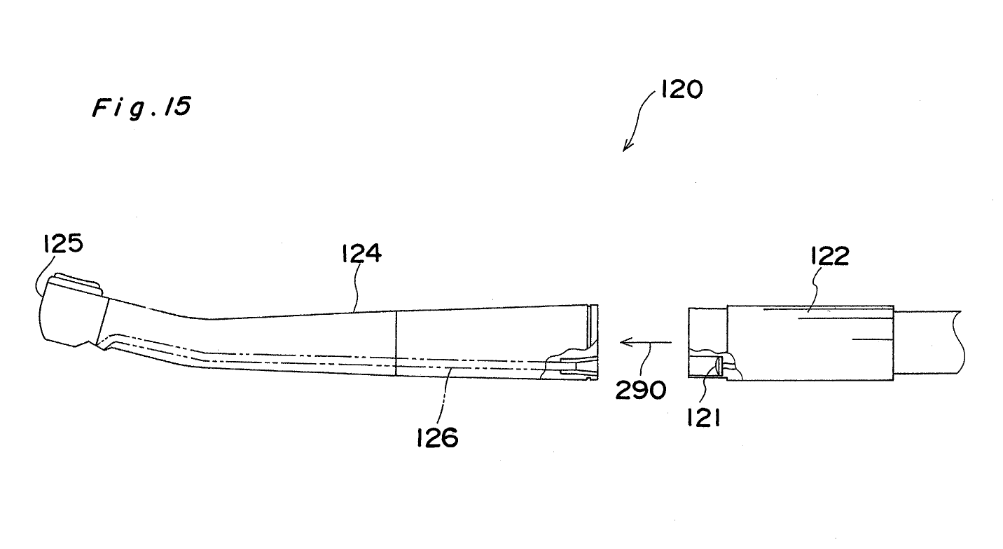

That is, FIG. 12 is a perspective view of a light emitting module 110 as an illumination apparatus for medical use; FIG. 13 is a front view thereof seen from a direction shown by an arrow 190 in FIG. 12; and FIG. 14 is a side view.

The light emitting module 110 is formed in approximately the same manner as the light emitting module 10 of FIG. 1, and a substrate 112 on which a plurality of bare chips 114 are arranged is covered with a resin mold 118.

Here, unlike the light emitting module 10 of FIG. 1, a lens plate 116 for leading out a beam of parallel light is arranged so as to be opposed to the bare chips 114 and is covered with a resin mold 118. The lens plate 116 has a plurality of lens elements 117 which are arranged in opposition to each of the bare chips 114 so that a spreading light beam emitted from the bare chips 14 is converted into a parallel beam of light. For example, a lens element 117 is a convex lens, and a light emitting portion of the bare chip 114 is arranged at a focal point of this convex lens.

Though the lens plate 116 and the bare chips 114 are located at a distance from each other in the figure, the lens plate 116 is, more preferably, made to contact the bare chips 114 in order to prevent the dispersion of light.

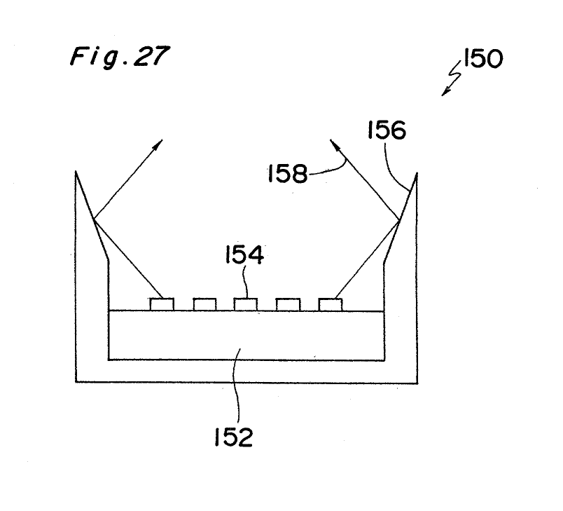

Alternatively, as a modification to the illumination apparatus shown in FIG. 27, a reflection plate 156 can be provided around the substrate 152 so that light emitted from the bare chips 154 is reflected thereby toward a center thereof as shown by an arrow 158. With the construction, the dispersion of light toward its peripheral area is prevented.

The lens plate 116, as shown in FIG. 12, allows parallel light to be emitted from the light emitting module 110, which can be appropriately utilized in a photopolymerizer for medical use, or in an instrument for medical use.

By making the lens elements 117 in an appropriate form or shape, it is possible to make parallel the light emitted from the light emitting module 10, to condense the light emitted therefrom, to disperse the light emitted therefrom, or to irradiate the light emitted therefrom at a predetermined angle.

FIG. 15 shows an embodiment as a hand piece 120 provided with a light emitting module 121. The light emitting module 121 is utilized for illumination in the oral cavity and, therefore, bare chips for emitting white light are used, in which they are constructed as shown in FIGS. 1, 12 through 14. The hand piece 120 is provided with a turbine head 124 and a coupling 122. The light emitting module 121 is provided on a side of the coupling 122, and the turbine head 124 is provided with a light guide 126. The coupling 122 is inserted into the turbine head 124 as shown by an arrow 290, so that, at the time of connection, light emitted from the light emitting module 121 passes through the light guide 126 and the light illuminates the vicinity of a tip of a tool for dental treatment attached to the head portion 125 of the turbine head 124.

The tool for dental treatment is driven by air supplied from outside. Employing the air, it is possible to cool the light emitting module 121.

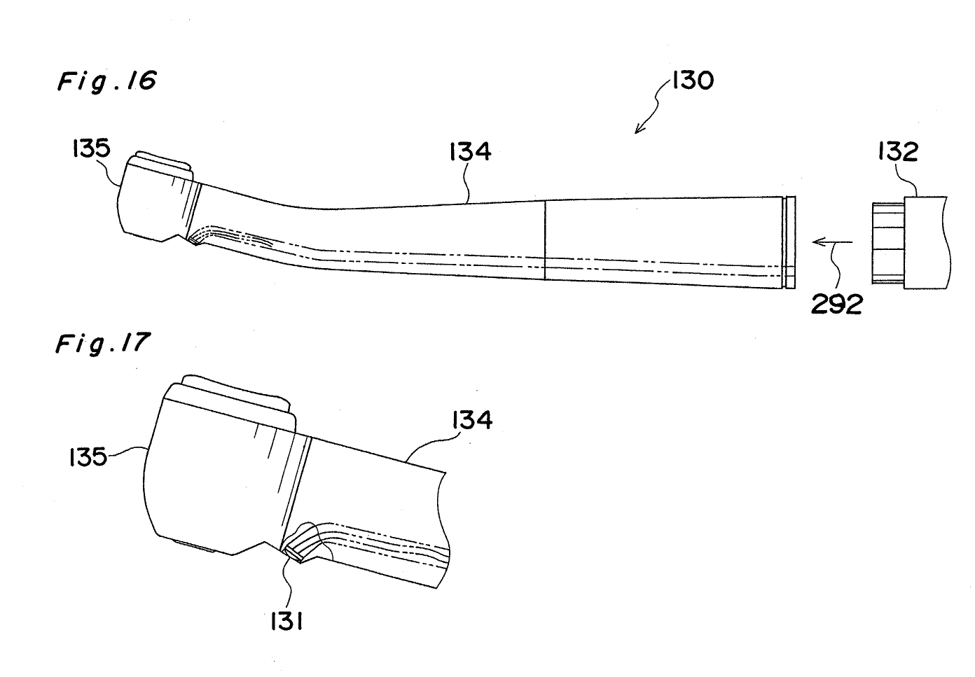

FIGS. 16 and 17 show an embodiment as a hand piece 130 provided with a light emitting module 131, in which the light emitting module 131 is provided on a side of the turbine head 134 of the hand piece 130. The light emitting module 131 is arranged in the vicinity of its head part 135 of the turbine head 134. When the coupling 132 is inserted into and is connected to the turbine head 134, as shown by an arrow 292, the electric power is supplied to the light emitting module 131 so that the vicinity of a tip of a tool for dental treatment mounted on its head part 135 can be illuminated.

The light emitting module 131 may be cooled down by means of the air that drives the tool for dental treatment.

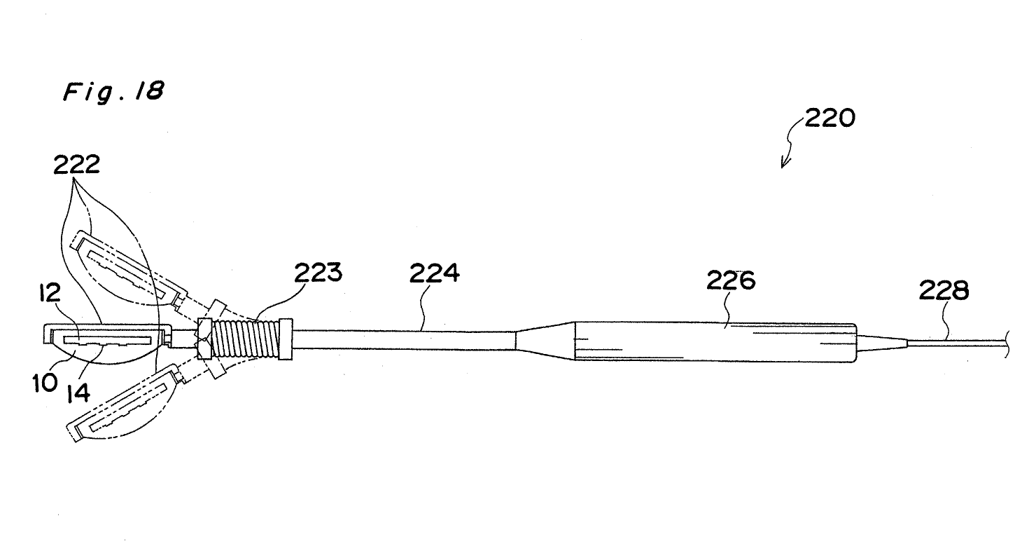

FIG. 18 shows an embodiment of a mirror-type photopolymerizer 220 for medical use provided with the light emitting module 10, in which a flexible part 223 is provided. A light output part 222, a supporter 224, a grip part 226 and an electric power supply cord 228, of the mirror-type photopolymerizer 220, are formed in the same manner as in the photopolymerizer 20 for medical use in FIG. 2. The light output part 222 is supported by the supporter 224 via the flexible part 223. The flexible part 223 has flexibility so that it can be bent by hand as shown by the chained lines in the figure and so that the bent condition can be maintained. By appropriately bending the flexible part 223, the grip part 226 can be held at an easily graspable angle so that light is allowed to be emitted in the desired direction.

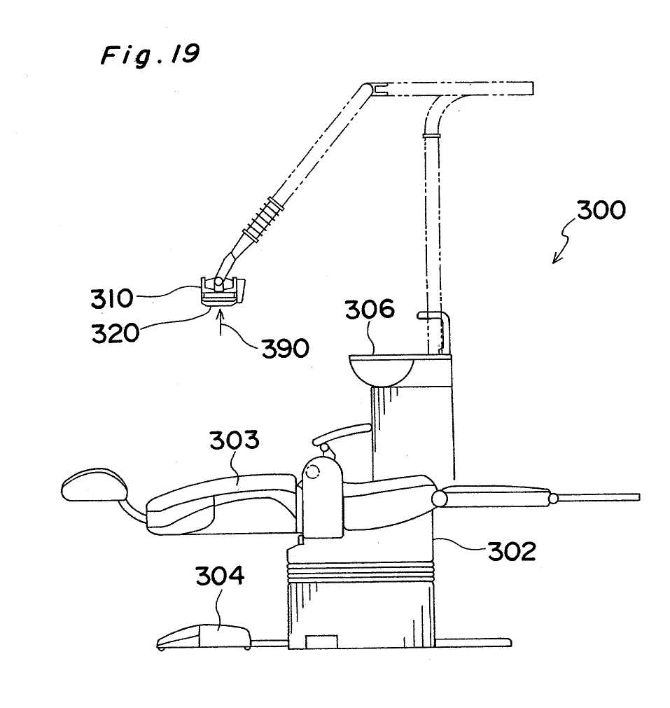

FIGS. 19 and 20 show an embodiment as a medical unit (or a dental unit) 300 provided with the light emitting module, in which the light emitting module formed by using bare chips, as shown in FIGS. 1, 12 through 14, is utilized for the illumination by the unit 300 for dental use.

The unit 300 for dental use is provided with a clinical chair 303 arranged on a base 302 so as to be capable of being freely lowered or raised, a spittoon 306, a light device 310 for illuminating the inside of the oral cavity, a foot controller 304 for foot operation, and the like.

As shown in FIG. 20 seen from the direction of an arrow 390, a pair of handles 314 are provided on two sides of a light part 320 which is in the center of the light device 310. A plurality of light emitting modules 321, 322, and 323 which emit blue light, and a plurality of light emitting modules 324, 325 and 326 which emit white light, are arranged in the light part 320. Since the light emitting modules 321, 322 and 323 which emit blue light, cover a broad illumination range, it can be used for photopolymerization in the entirety of the oral cavity or can be utilized for photopolymerization of craftwork(s) (object(s) prepared or made by a dental technician). The light emitting element modules 324, 325 and 326 which emit white light, are used for the illumination of the oral cavity. Switching of the illumination can be carried out by means of the foot controller 304.

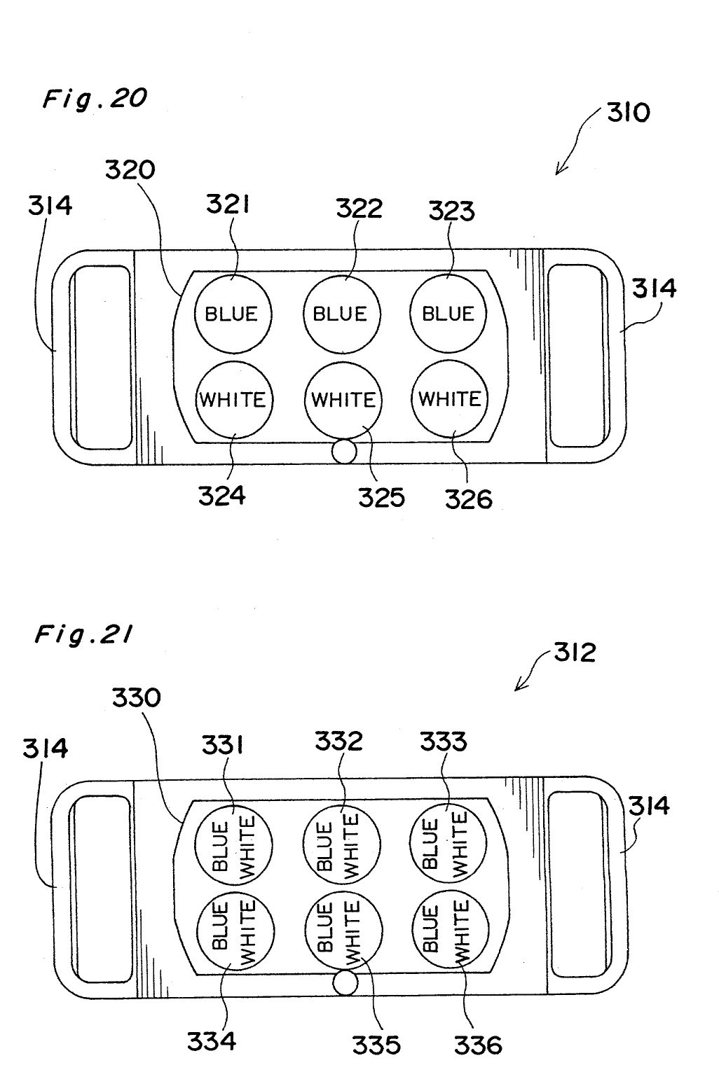

FIG. 21 shows a light device 312 according to a modification to the light device 310 of FIG. 20. In the construction, the plurality of light emitting modules 331 to 336 having bare chips for emitting white light and bare chips for emitting blue light arranged on the same substrate, are used for the light part 330 of the light device 312. Each of the light emitting modules 331 to 336 has two electrodes for the bare chips emitting white light and two electrodes for the bare chips emitting blue light. By selecting the electrodes for supplying the electric power through the operation of the foot controller 304, white light and blue light can be both emitted at the same time or one of them can be selected for emission.

The above described light emitting module 10, 110 is suitable as a light source for medical equipment, and it is possible to be miniaturized to a greater degree than the conventional light source.

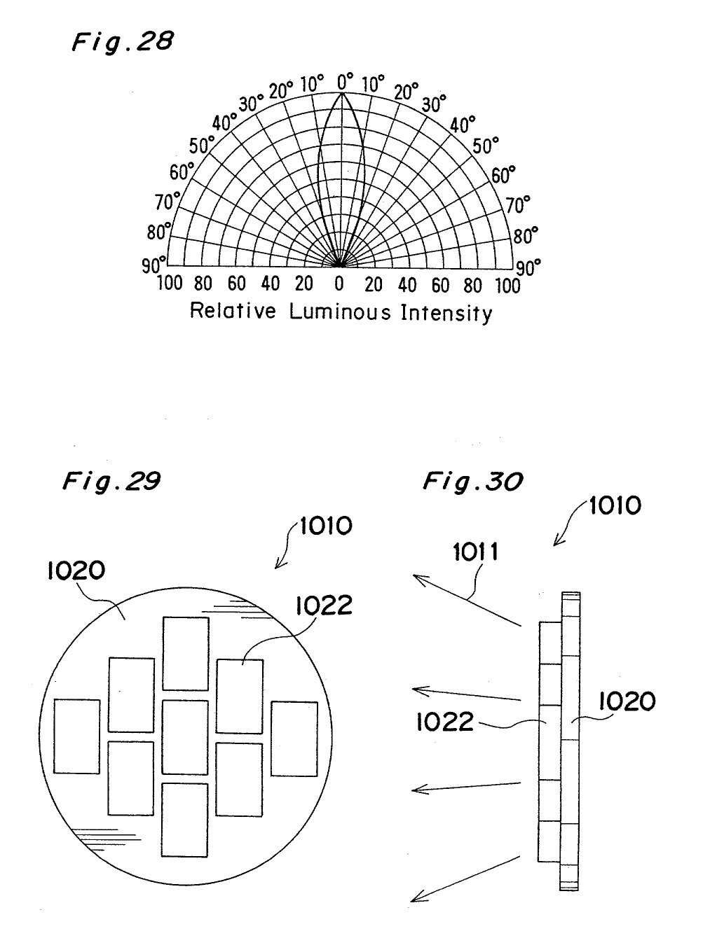

Next, with reference to FIGS. 28 through 45, it is explained about an illumination apparatus for medical use according to each of the fifth and sixth embodiments of the present invention, and about a photopolymerizer provided with the illumination apparatus.

That is, each of FIGS. 29 and 30 is a cross sectional view of a light emitting module 1010. A plurality of LED elements 1022 are collectively arranged on a substrate 1020 in the light emitting module 1010. Each of the LED elements 1022 used therein has a narrow directivity, of which the spread of light is narrow. Each thereof is, in addition, compact and of high performance having a great light emission. Light from the light emitting module 1010 spreads, as shown by, for example, arrows 1011, in compliance with the directivity of each LED element 1022.

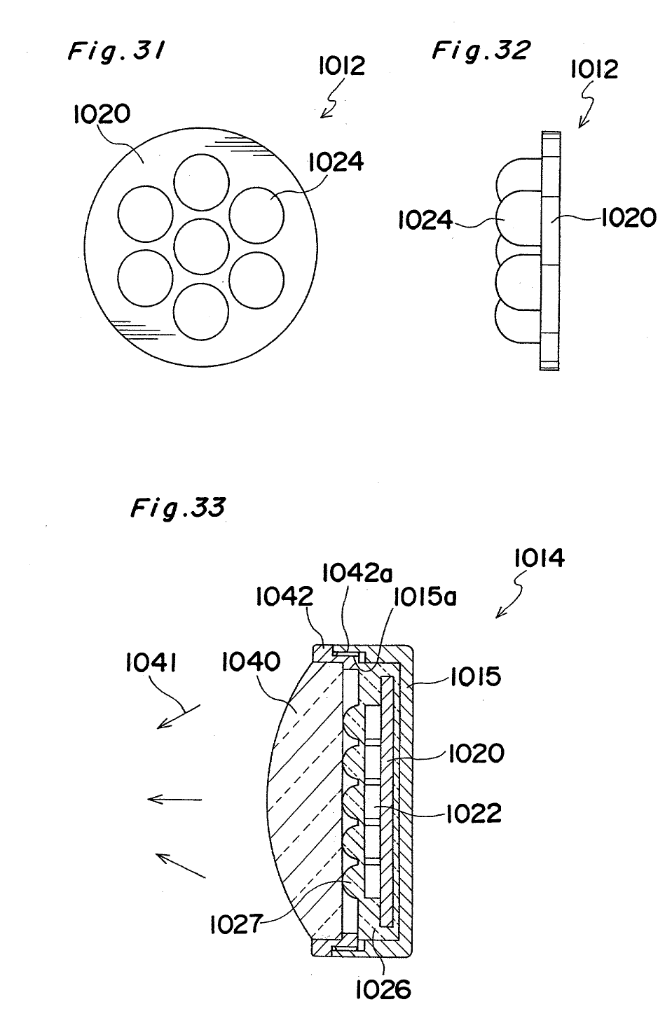

Therefore, LED elements 1022, of which the directivity is enhanced by condensing light through lenses housed within the packages, may be used as in the light emitting module 1012 shown in, for example, FIGS. 31 and 32.

In addition, in the case that the amount of light per unit area is insufficient, a greater amount of light can be, momentarily, gained by allowing a great amount of electric current to flow by means of a pulse drive. Thereby, a deep polymerization depth can be gained in the photopolymerization of, for example, a resin material for dental use.

Each of the light emitting modules 1010 and 1012 can be favorably utilized in a gun-type illumination apparatus.

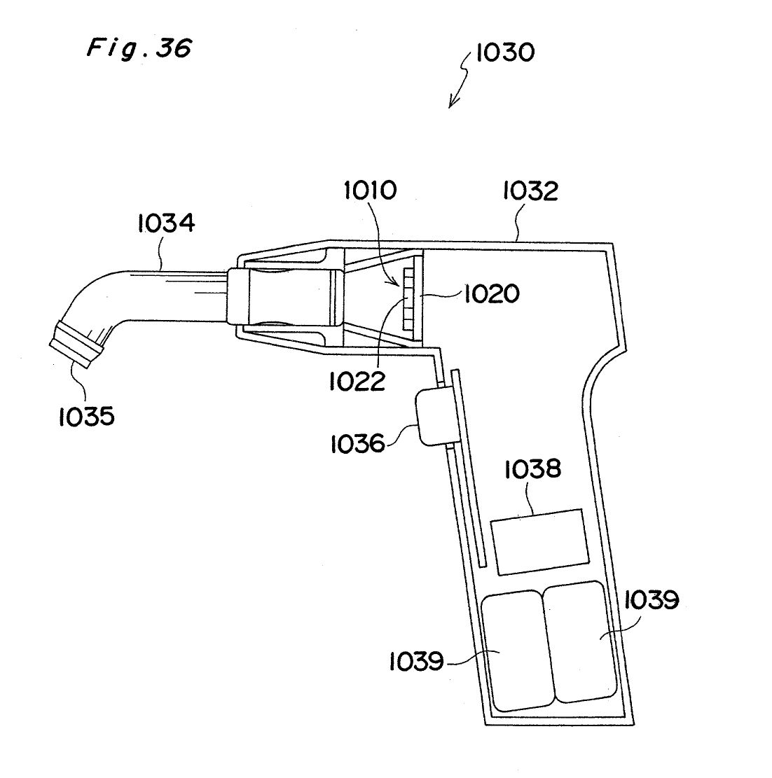

FIG. 36 shows an embodiment of the gun-type photopolymerizer 1030 provided with the light emitting module 1010. The photopolymerizer 1030 is preferable for, in particular, dental use. LED elements 1022 used in the light emitting module 1010 emit light (for example blue light) having a wavelength suitable for curing a photopolymerizing resin material (for example dental resin). LED elements 1022, which emit light of different wavelengths, are used for a photopolymerizing resin material gained by mixing a plurality of materials that are cured by differing wavelengths so that the respective materials are cured by the LED elements having different wavelengths.

In the photopolymerizer 1030 for medical use, a light guide 1034 is attached to an end portion of a housing 1032 in approximately an L-shape. The light emitting module 1010 is arranged so as to be opposed to one end surface of the light guide 1034 within the housing 1032 so that light is emitted from the other end surface 1035 of the light guide 1034. The photopolymerizer 1030 for medical use is of a cordless-type in which a control circuit substrate 1038 and electric power supply batteries 1039 are accommodated within the housing 1032 so that the LED elements 1022 in the light emitting module 1010 emit light when an operational switch 1036 protruding from the housing 1032 is pressed.

The light guide 1034, which has a high possibility of coming into contact with the teeth, can be sterilized after being removed from the photopolymerizer 1030 for medical use. In addition, a variety of types of light guides 1034 with different curved forms, sizes, or the like, can be prepare and selected, in accordance with the purpose of utilization, and any particular one of them can be mounted to the photopolymerizer.

Furthermore, a lens may be provided between the light emitting module 1010 and the light guide 1034 in order to enhance the condensing characteristics and/or in order to utilize light more efficiently. In this case, the lens can be made removable so that it can be exchanged with a lens of appropriate condensing characteristics so as to correspond to, for example, the light guide 1034, which is mounted to the photopolymerizer.

Or, a plurality of LED elements are aligned on a curved substrate so that light is emitted from the individual LED elements toward a common point, and the plane of incidence of the light guide is arranged at the common point. Or, the LED elements are aligned having angles on a planar substrate so that light is emitted from the individual LED elements toward a common point, and the plane of incidence of the light guide is arranged at the common point.

Next, with reference to FIGS. 33 through 35, 37, it is explained about an illumination apparatus for medical use according to the sixth embodiment of the present invention, and a mirror-type photopolymerizer provided with the illumination apparatus.

That is, FIG. 33 is a cross sectional view of a light emitting module 1014. In the light emitting module 1014, a substrate 1020, a plurality of LED elements 1022 and a resin mold 1026 are arranged in a housing 1015. In the resin mold 1026, a portion 1027 that is opposed to each of LED elements 1022 is formed in an appropriate shape, such as a concave lens form or a convex lens form, so that light from the LED elements 1022 is condensed. For example, light emitted from the LED elements 1022 is converted to parallel light. A condensing lens 1040 is arranged so as to be opposed to the resin mold 1026 in order to condense light as shown by arrows 1041.

The condensing lens 1040 is held by a holding frame 1042. An external thread 1042a is provided around the holding frame 1042 so as to be engaged with an internal thread 1015a provided inside the housing 1015. Thereby, the condensing lens 1040, which has a possibility of coming into contact with the teeth, or the like, can be removed for sterilization or can be exchanged with another lens of appropriate condensing characteristics.

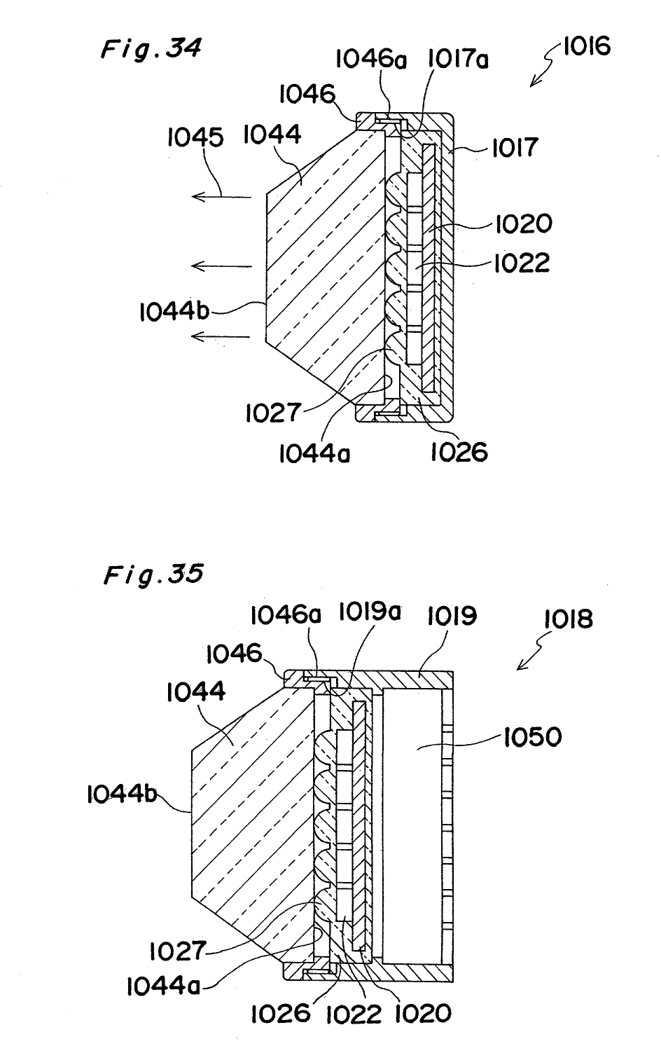

FIG. 34 is a cross sectional view showing a light emitting module 1016 according to a modification. The light emitting module 1016 is formed in approximately the same manner as the light emitting module 1014 in which a substrate 1020, a plurality of LED elements 1022 and a resin mold 1026 are arranged within a housing 1017, and portions 1027 opposed to the respective LED elements 1022 are formed so as to condense light from the LED elements 1022 in the resin mold 1026.

Here, the light emitting module 1016 differs from the light emitting module 1014 in that a tapered light guide 1044 is arranged so as to be opposed to the resin mold 1026. The tapered light guide 1044 is formed by, for example, bundling a plurality of optical fibers into a tapered form in which the side of the plane of incidence 1044a opposed to the resin mold 1026 is greater than the side of the plane of light irradiation 1044b. By mounting the tapered light guide 1044 to the light emitting module, a narrow range can be intensively irradiated with light of a high brightness. For example, in the case of the tapered light guide 1044 having a length of 10 mm, in which the plane of incidence 1044a has a diameter of 15 mm and the plane of light irradiation 1044b has a diameter of 8 mm, the amount of light per unit volume can be expected to approximately triple.

The tapered light guide 1044 is held in a holding frame 1046 in the same manner as the condensing lens 1040. An external thread 1046a is provided outside around the holding frame 1046 so as to be engaged with an inner thread 1017a provided inside the housing 1017. Thereby, the tapered light guide 1044, which has a possibility of coming into contact with the teeth, can be removed for sterilization or can be exchanged with another light guide of appropriate condensing characteristics.

FIG. 35 is a cross sectional view of a light emitting module 1018 according to a modification. The light emitting module 1018 is formed in approximately the same manner as the light emitting module 1016 in which a substrate 1020, a plurality of LED elements 1022 and a resin mold 1026 are arranged within a housing 1019. In addition, a tapered light guide 1044 is attached to the housing 1019 via a holding frame 1046 so as to be opposed to the resin mold 1026.

Here, unlike the light emitting module 1016, a fan 1050 is housed within the housing 1019 so as to make air strike the substrate 1020. In the case that the heat emission of the LED elements 1022 cannot be ignored such as in the case that, for example, the LED elements 1022 are integrated in a great number or the output of the LED elements 1022 is great, the heat generated by the LED elements 1022 can be efficiently dissipated (or discharged).

The light emitting modules 1014, 1016 and 1018 provided with the condensing lens 1040 or the tapered light guide 1044 can narrow the directivity of light and, therefore, can be favorably utilized in a mirror-type illumination apparatus. It can, of course, be utilized favorably in a gun-type illumination apparatus.

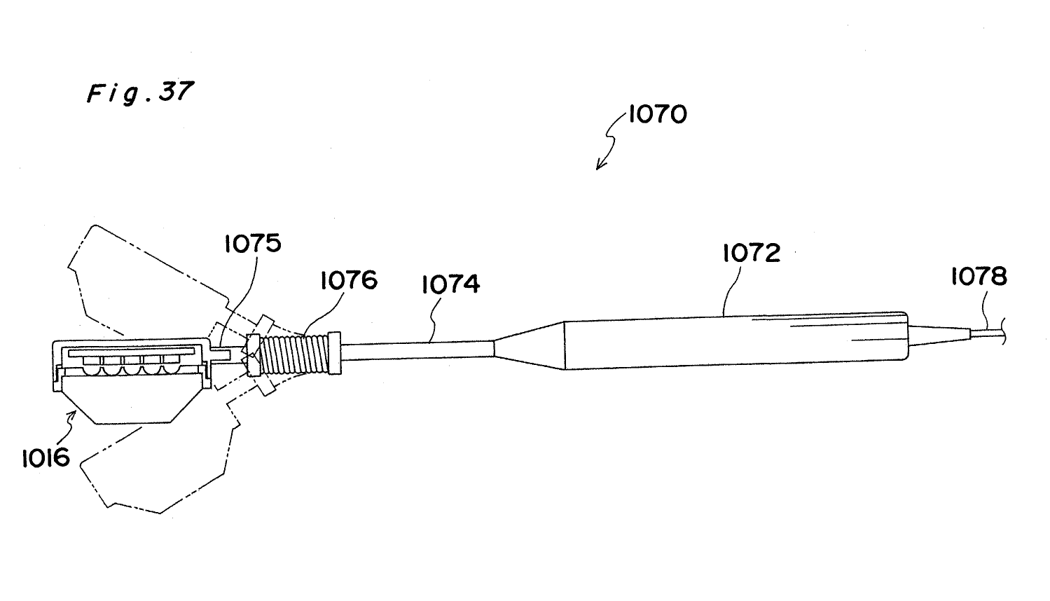

FIG. 37 shows an embodiment of a mirror-type photopolymerizer 1070 for dental use provided with the light emitting module 1016. The light emitting module 1016 is supported by one end 1075 of a supporter 1074 having a long and narrow axis form. The other end of the supporter 1074 is secured to a grip part 1072 which can be gripped by hand. An electric power supply cord 1078 for supplying electric power to the light emitting module 1016 is connected to the grip part 1072. In the case that the light emitting module 1016 is thin, it can easily be placed in a gap, or the like, within the oral cavity and is, therefore, this is convenient.

A flexible part 1076 is provided to the supporter 1074. The flexible part 1076 has flexibility to the degree that it can be bent by hand as shown in by the chained lines and the bent condition can be maintained. The flexible part 1076 may be a component in which a plurality of parts are connected under an appropriate binding force, such as an arm of an electric lamp, or may be formed of an elastic and flexible material as described above. Light can be emitted in a desired direction with the grip part 1072 being gripped at an easily graspable angle by appropriately bending the flexible part 1076.

As described above, light with high output power can be emitted by using the plurality of LED elements.

Here, the present invention is not limited to the above described embodiments, but, rather, can be implemented in a variety of other forms or modifications.

For example, the light emitting module 1014 of FIG. 33 may be used in the gun-type photopolymerizer of FIG. 36. In this case, light condensed in the light guide 1034 can be emitted in a state in which the convergence of light is enhanced. Also, a narrow light guide 1034 can be used.

Alternatively, the illumination device may have a construction in which the light emitting module is cooled down.

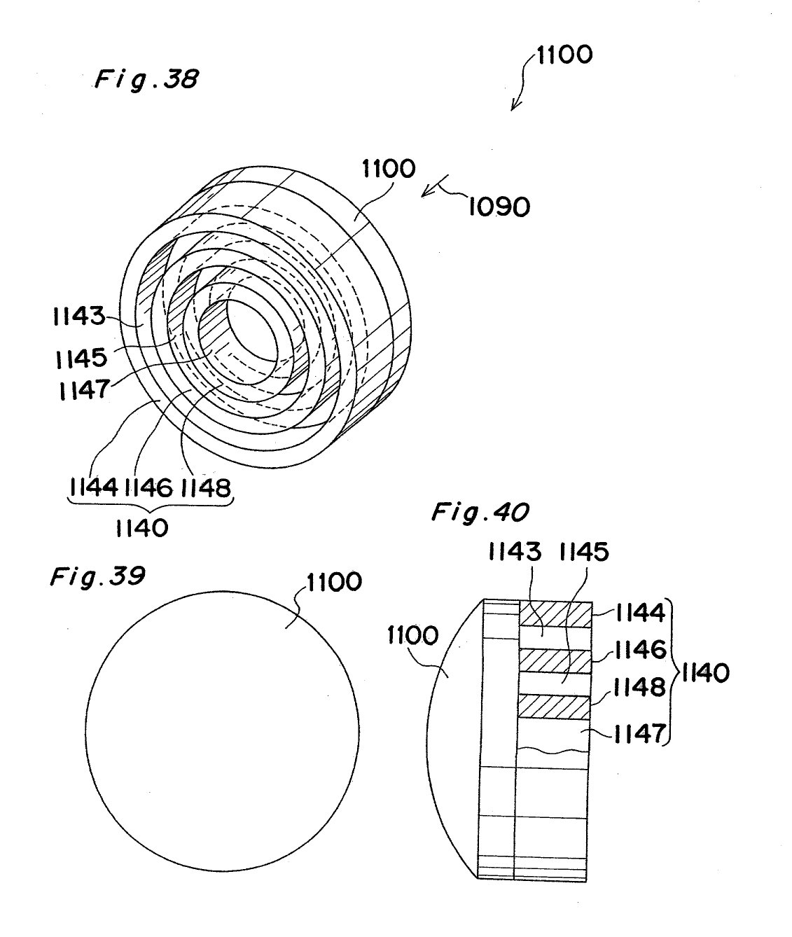

FIGS. 38 to 40 show an illumination apparatus for medical use in which a light emitting module 1100 and a heat sink 1140 are connected to each other. FIG. 38 is a perspective view seen from the substrate side of the light emitting module; FIG. 39 is a front view thereof seen from the direction (LED elements side) shown by an arrow 1090 in FIG. 38; and FIG. 40 is a side view of FIG. 38. The light emitting module 1100 is formed in the same manner as the above described light emitting modules 1010, 1012, 1014 and 1016. The heat sink 1140 is attached to the substrate side of the light emitting module 1100 so as to radiate the heat generated by the light emitting module 1100. The heat sink 1140 is provided with a plurality of fins 1144, 1146 and 1148. The fins 1144, 1146 and 1148 are formed in a concentric cylindrical form so as not to damage the oral cavity, or the like, even when contact is made therebetween. In addition, spaces 1143, 1145 and 1147 are formed between the fins 1144, 1146 and 1148 so as to increase the area of heat radiation.

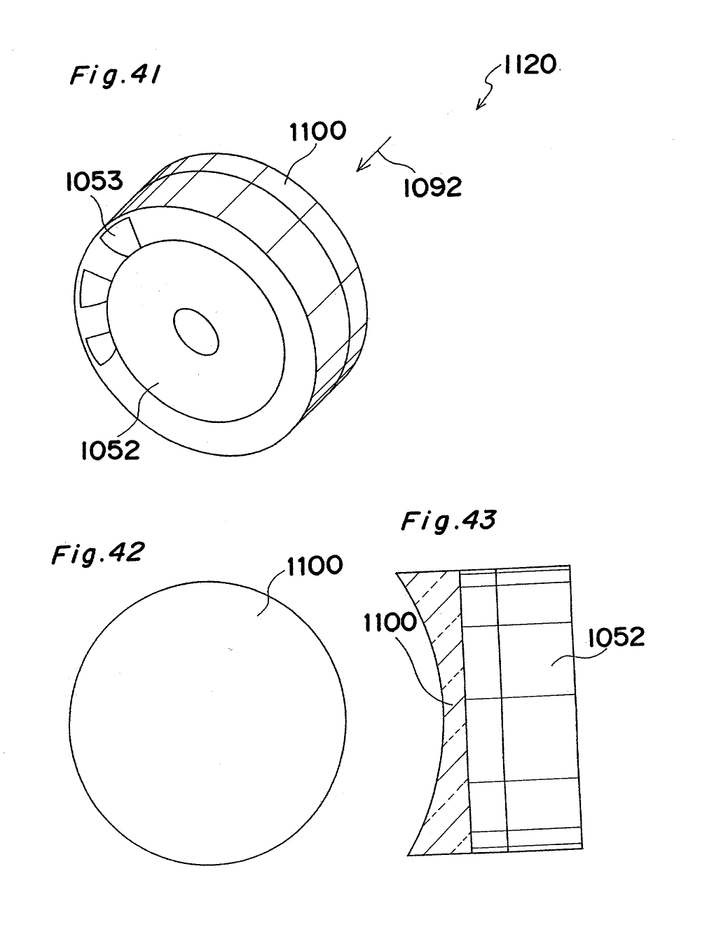

FIGS. 41 through 43 show an illumination apparatus for medical use 1120, in which a light emitting module 1100 and a fan 1052 are combined with each other. FIG. 41 is a perspective view thereof seen from the substrate side of the light emitting module 1100; FIG. 42 is a front view thereof seen from the direction shown by an arrow 1092 in FIG. 41; and FIG. 43 is a partially broken side view of FIG. 41. The fan 1052 is attached to the substrate side of the light emitting module 1100. The fan 1052 cools the light emitting module 1100 by making air strike it by means of a plurality of rotating blades 1053 (only three blades are shown in the figure, and the others are omitted).

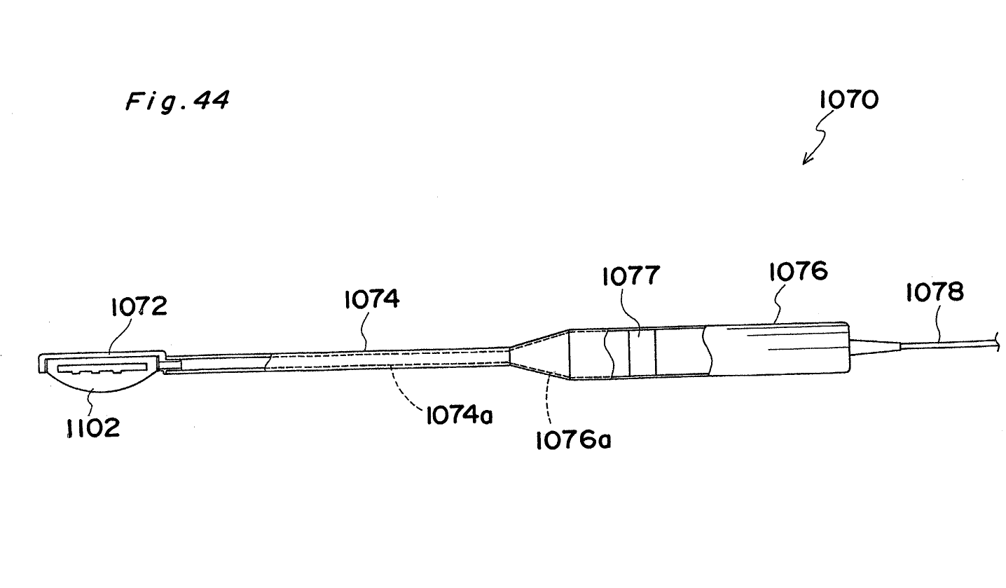

FIG. 44 shows an embodiment of a mirror-type photopolymerizer provided with the illumination apparatus for dental and medical use. In the construction, the light emitting module 1100 is cooled down by providing a fan 1077 mounted inside the grip part 1076 of the mirror-type photopolymerizer 1070. Each of the supporter 1074 and the grip part 1076 is formed hollow, in which paths 1074a and 1076a for allowing air from the fan 1077 to flow are arranged therein. In the construction, the air for cooling is sent (or forwarded or supplied) to the light emitting module 1102 attached to the light output part 1072. The electric power is supplied to the fan 1077 from an electric power supply cord 1078.

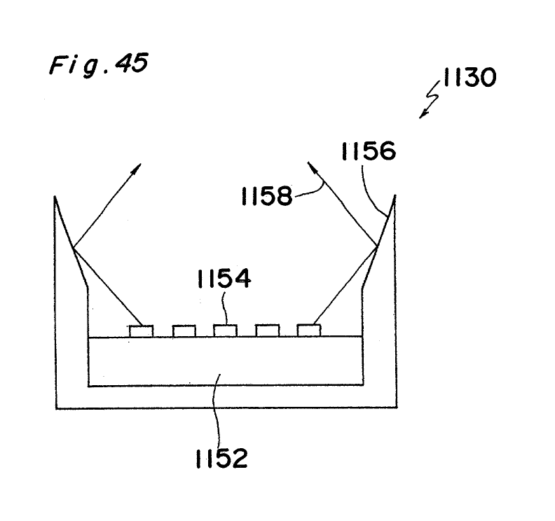

Alternatively, a reflection plate 1156 may be provided around the substrate 1152 as in the light emitting element module 1130, as an illumination apparatus, as shown in FIG. 45. With the construction, light emitted from the LED elements 1154 are reflected toward a center thereof as shown by arrows 1158 in a state in which the dispersion of light toward its peripheral area is prevented.

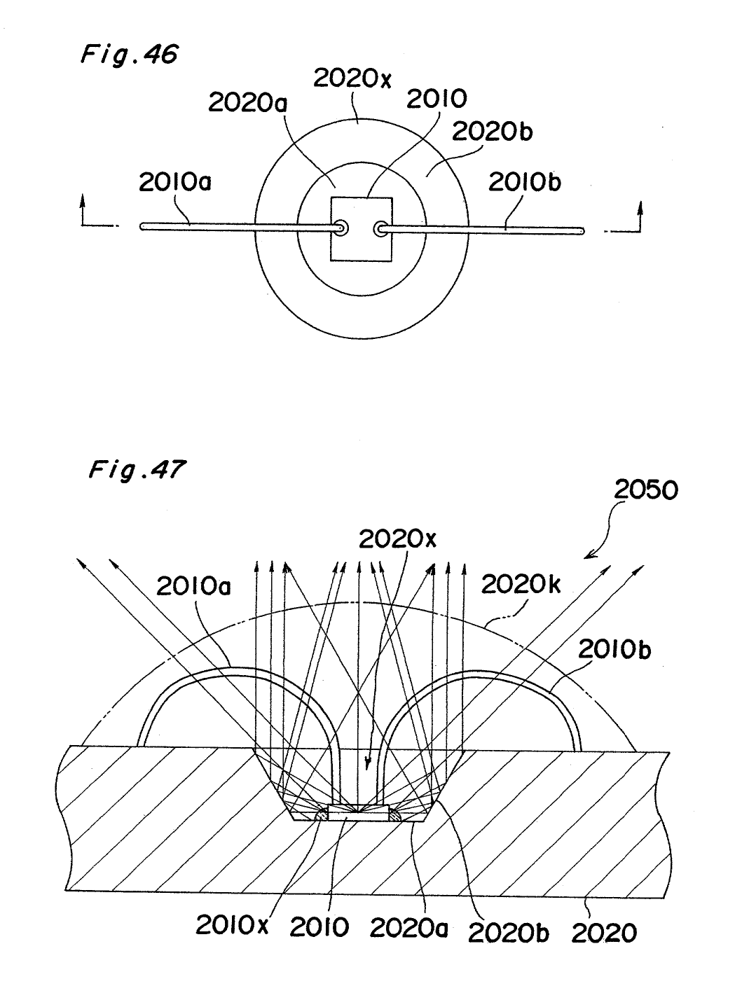

Next, with reference to FIGS. 46 through 96, it is explained about an illumination apparatus for medical use, as a light emitting module, according to each of the seventh through twentieth embodiments of the present invention, and a photopolymerizer provided with the illumination apparatus.

The illumination apparatus can be formed in a variety of embodiments, as shown in FIGS. 46 to 74.

That is, the illumination apparatus 2050, as a light emitting module, according to the seventh embodiment shown in FIGS. 46 and 47, is generally provided with a light emitting element 2010 and a substrate 2020, as a supporting member, to support the light emitting element 2010. A recess 2020x is formed on the substrate 2020, and the light emitting element 2010 is placed on the bottom 2020a of the recess 2020.

A ceramic or glass epoxy substrate, for example, is used for forming the substrate 2020, and the recess 2020x is formed by sintering, after machining or after molding. The light emitting element 2010 is a bare chip of a light emitting diode (LED) and is fixed as shown in FIG. 47 by means of a fixing agent, for example, by silver paste 2010x. The light emitting element 2010 is connected to the wiring pattern of the substrate 2010 by means of wires 2010a and 2010b so as to emit light when a voltage is applied. The light emitting element 2010 and the wires 2010a and 2010b are protected by a resin mold 2020k if necessary. In addition, the resin mold 2020k has the effect of increasing the amount of light. A silicon resin or an epoxy resin or a resin gained by appropriately combining a plurality of types of resins is used for the resin mold 2020k.

The bottom 2020a and the sides 2020b of the recess 2020x are formed so as to have a high reflectance. In the construction, the light from the emitting element 2010 is efficiently reflected by the sides relative to the light emitting element 2010 (right and left parts in the figure) and by the rear side relative thereto (lower part in the figure), towards its front (upward in the figure).

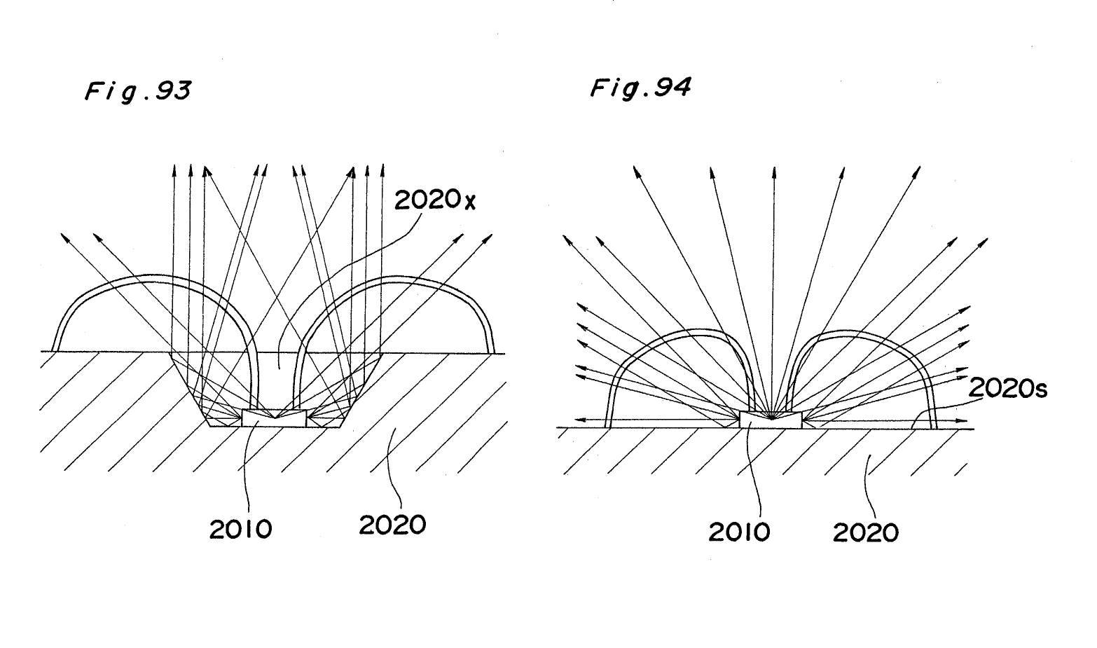

In the case that the light emitting element 2010 is fixed to the recess 2020x of the substrate 2020 as shown in FIG. 93, a greater amount of light can be collected to the front (upward in the drawing) of the light emitting element 2010 in comparison with the case in which the light emitting element 2010 is fixed to the plane 2020s of the substrate 2020 as shown in FIG. 94.

Here, a glass epoxy substrate, a ceramic substrate, an alumina substrate, or a substrate in which a metal plate is coated with an insulator, can be used for the substrate 2020, and the recess 2020x can be formed by an appropriate method in accordance with the type of the substrate 2020. Also, it is possible to use a bare chip such as a laser semiconductor (or semiconductor for emitting a laser beam) or organic EL (electroluminescence), as the light emitting element 2010. It is preferable to use a material, as the substrate 2020, that can easily dissipate the heat generated by the light emitting element 2010.

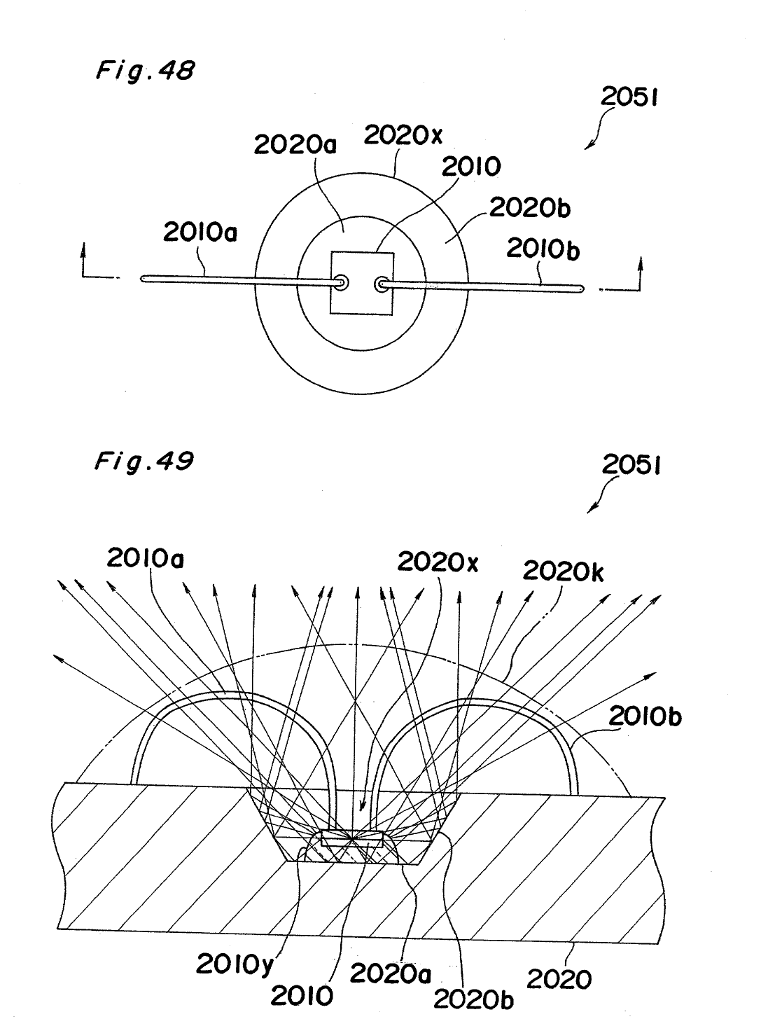

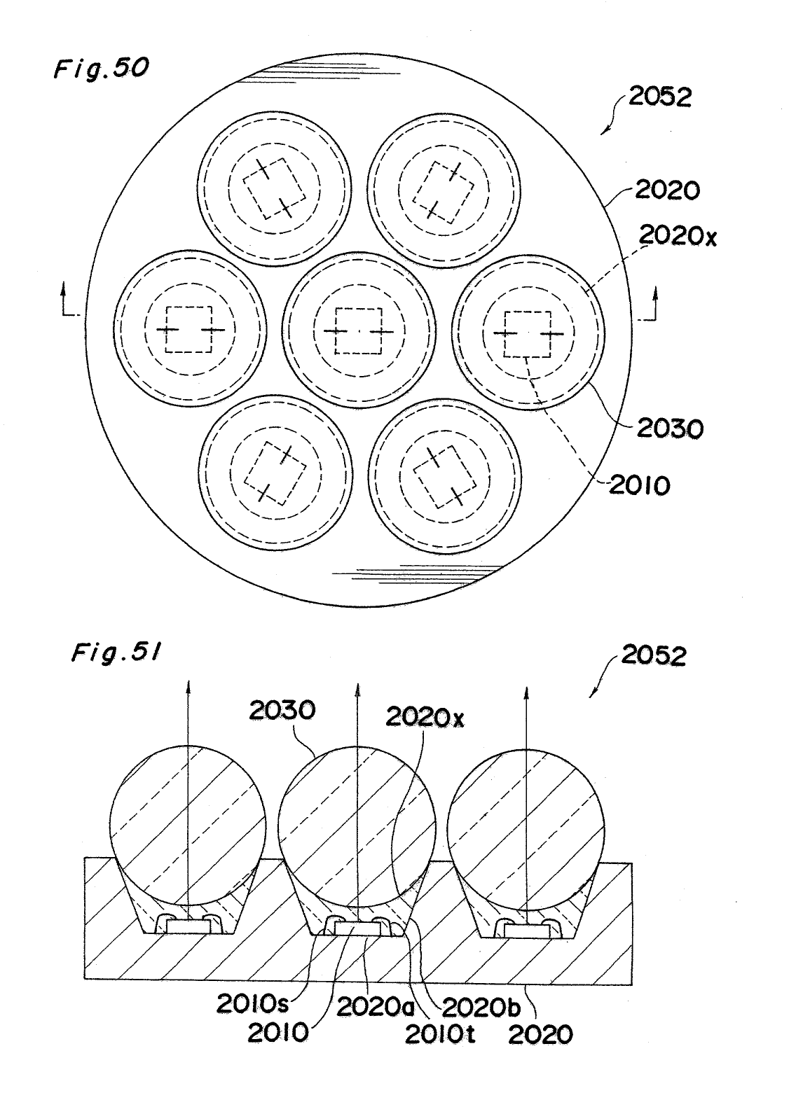

FIGS. 48 and 49 show an illumination apparatus 2051, as a light emitting module, according to the eighth embodiment. As shown in the figure, the light emitting element 2010 is fixed so as to be raised off from the bottom 2020a of the recess 2020x of the substrate 2010. The other parts of the configuration are the same as that of the illumination apparatus 2050 of FIGS. 46 and 47. In the illumination apparatus 2051, most of the light emitted in the backward direction from the light emitting element 2010 is reflected on the bottom 2020a so that the light travels towards the front and, therefore, the amount of light that travels toward the front of the light emitting element 2010 is greater than in the illumination apparatus 2050 of FIGS. 46 and 47. Namely, the condensing characteristics of light can be enhanced with the construction.

FIGS. 50 and 51 show an illumination apparatus 2052, as a light emitting module, according to the ninth embodiment. As shown in the figure, the illumination apparatus 2052 has a plurality of ball lenses 2030 for enhancing the light condensing characteristics. The ball lenses 2030 are fixed to recesses 2020x of the substrate 2020 by a transparent fixing agent filled into the recesses 2020x. As the transparent fixing agent, for example, a resin mold such as an epoxy resin, a silicon resin, a resin in which a plurality of types of resins are appropriately combined, or the like, can be employed. Since the ball lenses 2030 do not have directivity, they are easy to position relative to the recesses 2020x or to light emitting elements 2010, with a high precision.

Though in the illumination apparatus 2052 of FIGS. 50 and 51, a plurality of recesses 2020x are provided in the substrate 2020 so that light emitting elements 2010 are placed into the respective recesses, there may be only one pair of recesses each of which the light emitting element 2010 is installed in. Also, though wires 2010s and 2010t connected to the light emitting elements 2010 are arranged within the recesses 2020x, they may protrude from the recesses 2020x.

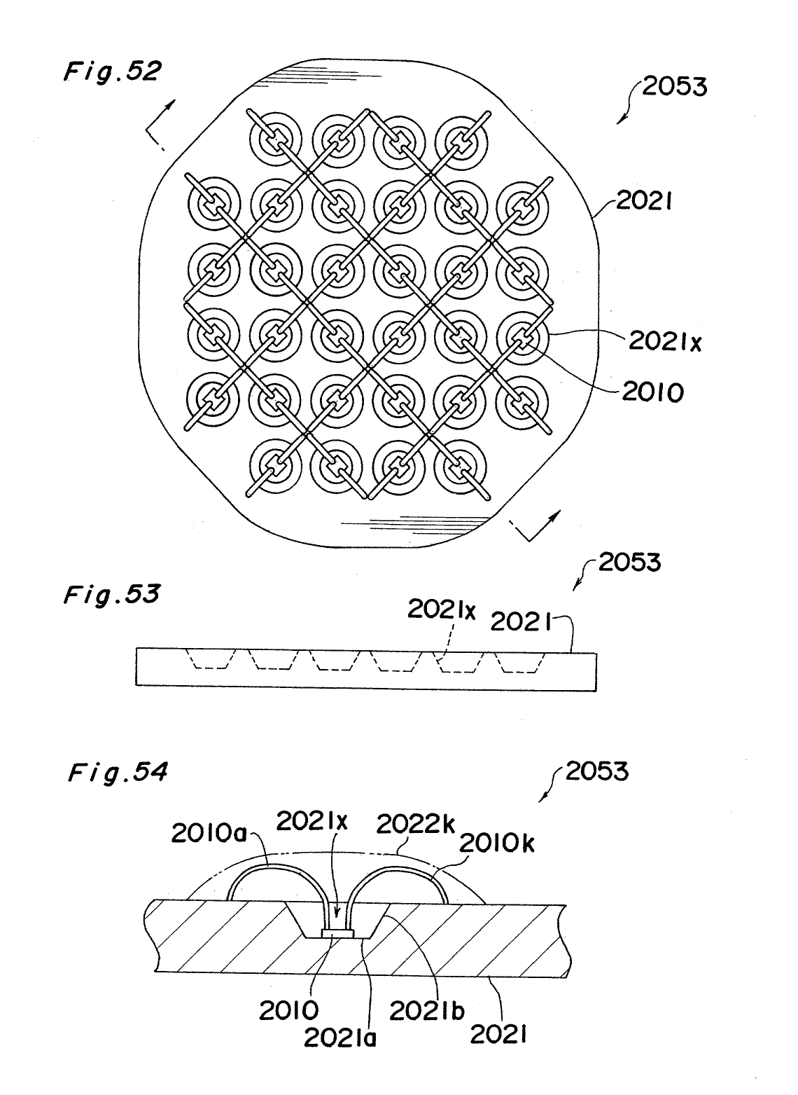

FIGS. 52 and 53 show an illumination apparatus 2053, as a light emitting module, according to the tenth embodiment. As shown in the figure, a plurality of recesses 2021x are provided on a substrate 2021 so that one light emitting element 2010 is placed in each of the respective recesses. The basic configuration in which a light emitting element 2010 is placed in a recess 2021x is the same as that of the illumination apparatus 2050 of FIGS. 46 and 47.

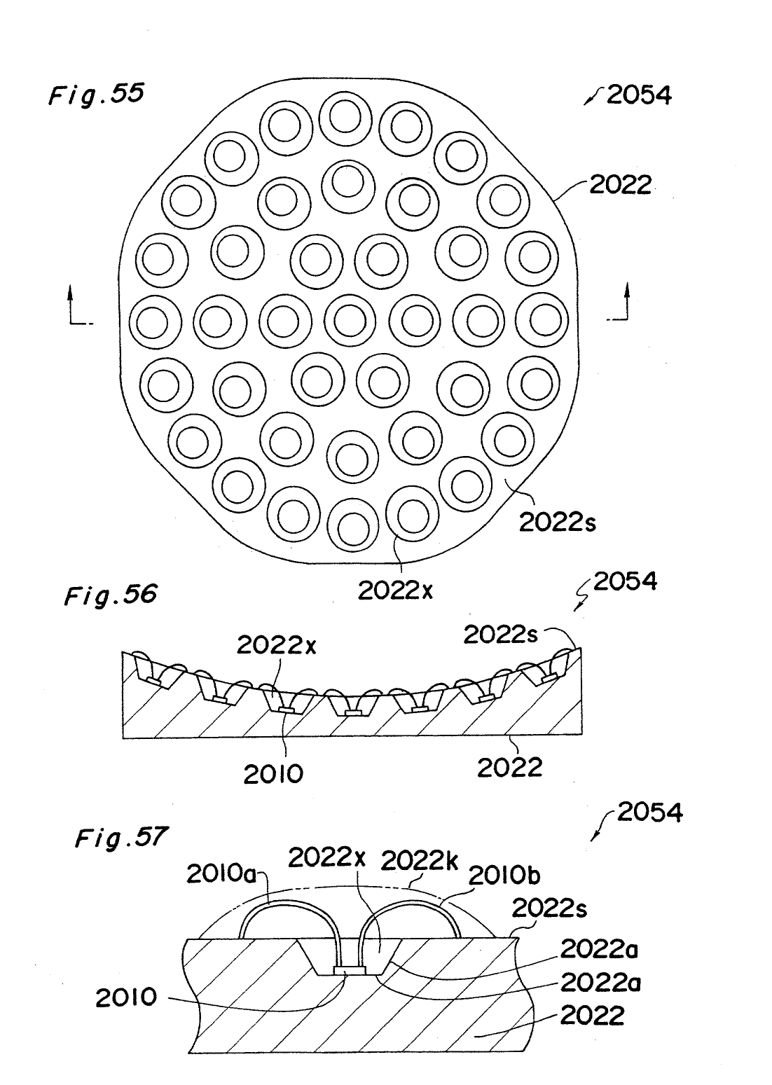

FIGS. 55 through 57 show an illumination apparatus 2054, as a light emitting module, according to the eleventh embodiment. As shown in the figure, a plurality of recesses 2022x are provided on a curved surface 2022s of the substrate 2022 so that one light emitting element 2010 is placed in each of the respective recesses. The basic configuration in which a light emitting element 2010 is placed in a recess 2022x is the same as that of the illumination apparatus 2050 of FIGS. 46 and 47. Here, in FIG. 55, the light emitting elements 2010 and the wires 2010a and 2010b are omitted in the figure.

Since the bottom surfaces 2022a of the recesses 2022x are formed so as to be gradually tilted along the curved surface 2022s of the substrate 2020, light from the respective light emitting elements 2010 arranged in the recesses 2022x can be collected in the vicinity of the center of curvature of the curved surface 2022s.

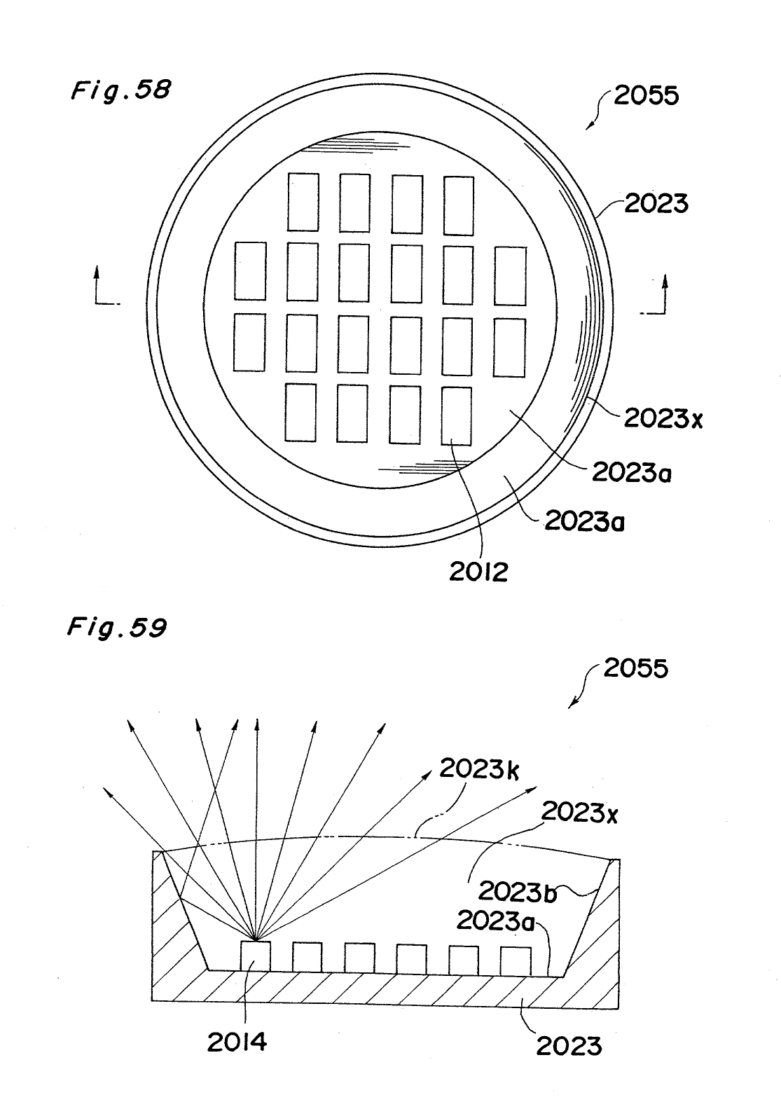

As shown in FIGS. 58 to 61, a plurality of light emitting elements may be arranged inside a recess.

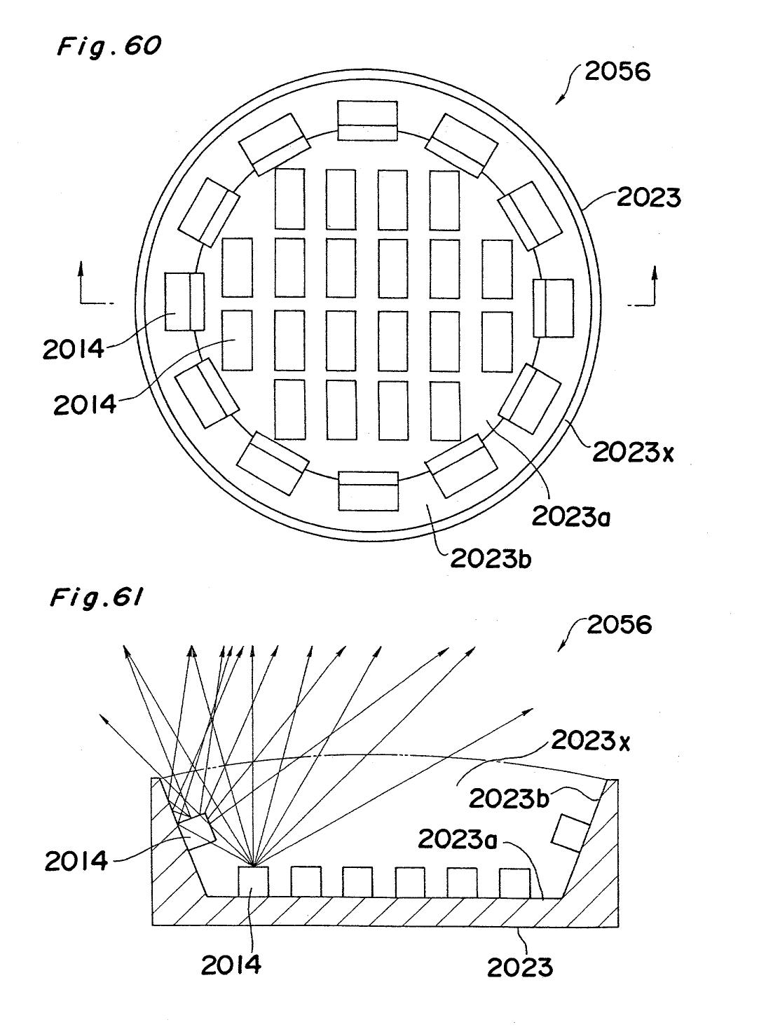

That is, FIGS. 58 and 59 show an illumination apparatus 2053, as a light emitting module, according to the twelfth embodiment. As shown in the figure, a plurality of light emitting elements 2014 are arranged on the bottom surface 2023a of a single recess 2023x formed in the center of a substrate 2023. A resin mold 2023k, or the like, may be filled into the recess 2023x. The side surface 2023b of the recess 2023x reflects light that has traveled sideways from the light emitting elements 2014, so that the light is directed towards the front as shown in FIG. 59. Thereby, the condensing characteristics of the light emitted from the light emitting elements 2014 is enhanced.

By the way, FIGS. 58 and 59 show a case in which the light emitting elements 2014 are chip LEDs (LED devices in which bare chips are housed in packages). Since a chip LED has a high directivity of light, only the side surface 2023b can be formed as a reflecting surface as shown in the figure.

In the construction, the side surface 2023b is formed with a cross section thereof being an ellipse or a parabola. Thereby, the reflected light is allowed to travel towards the front, in a parallel manner, in a condensing manner, or in a spreading manner, or light.

FIGS. 60 and 61 show an illumination apparatus 2056, as a light emitting module, according to the thirteenth embodiment. As shown in the figure, light emitting elements 2014 are additionally arranged on the side surfaces 2023b of the recess 2023x. In the illumination apparatus 2056, there can be provided a large number of the light emitting elements 2014, thus achieving an increase in amount of the light.

Here, it is possible to provide a plurality of configurations each of which is as shown in FIGS. 58 to 61, on a single substrate.

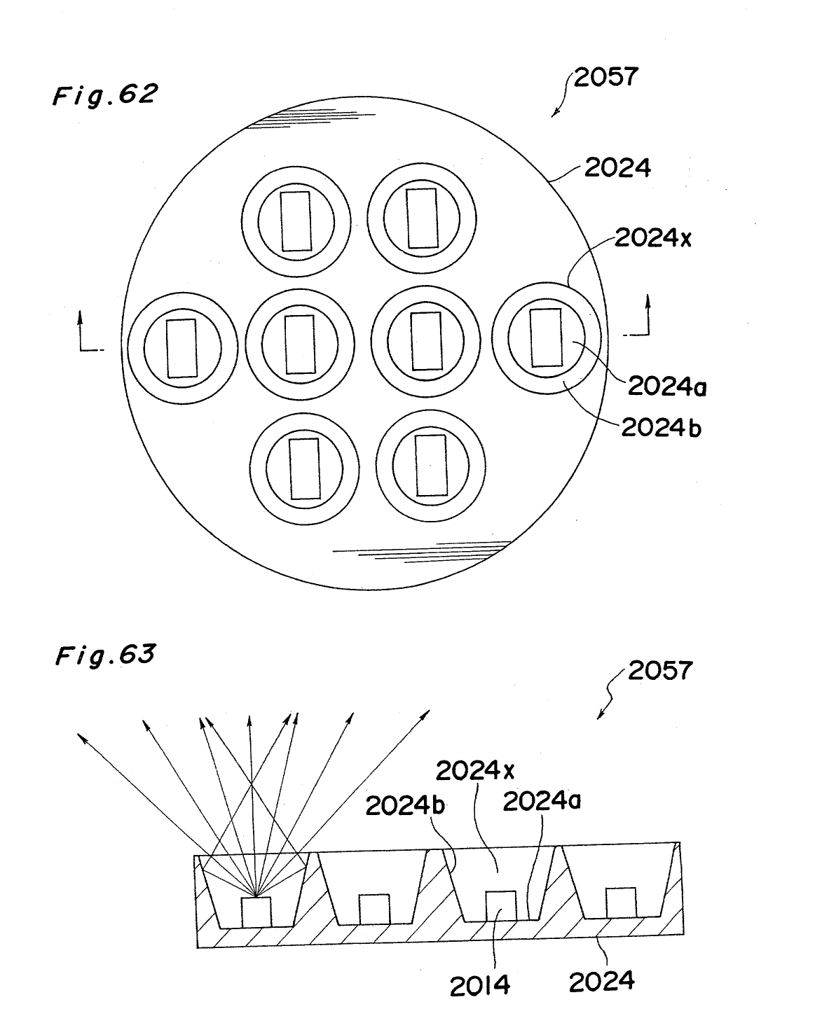

FIGS. 62 and 63 show an illumination apparatus 2057, as a light emitting module, according to the fourteenth embodiment. As shown in the figure, light emitting elements 2014 of chip LEDs are arranged on the bottom surfaces 2024a of the recesses 2024x formed on the substrate 2024. Even though a chip LED 2014 has a high directivity, light that has traveled in a direction away from the front can be reflected towards its frontal direction by reflection on the side surface 2024b of a recess 2024x. With the construction, an increase in the amount of light can be achieved.

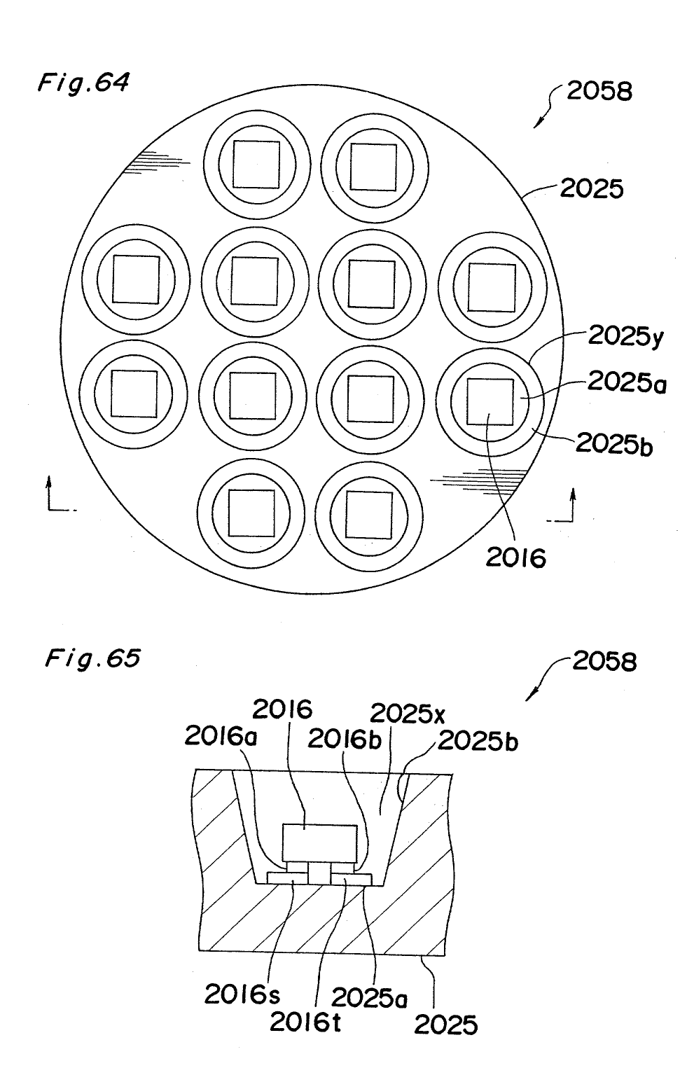

FIGS. 64 and 65 show an illumination apparatus 2058, as a light emitting module, according to the fifteenth embodiment. As shown in the figure, the illumination apparatus 2058 has a bonding-less structure (or non-bonding structure). In a light emitting element 2016, an electrode 2016a on the cathode side and an electrode 2016b on the anode side are formed of solder bumps, or of gold bumps, on one side of a bare chip. Wiring patterns 2016s and 2016t are formed on the bottom surface 2025a of a recess 2025x. The electrodes 2016a and 2016b of the light emitting element 2016, and the wiring patterns 2016s and 2016t, are, respectively, connected through thermal solder reflow or through ultrasonic vibration under pressure. A resin may be molded in the recess 2025x so that the light emitting element 2016 is protected and fixed therein. The resin mold makes possible treatment (sterilization process in a high temperature steam) in an autoclave, under a condition in which an electrical wiring portion is not exposed.

The aforementioned lens, resin and other components can, of course, be combined in different ways The number of light emitting element(s) may be one, or may be plural. Any type of light emitting elements may be used.

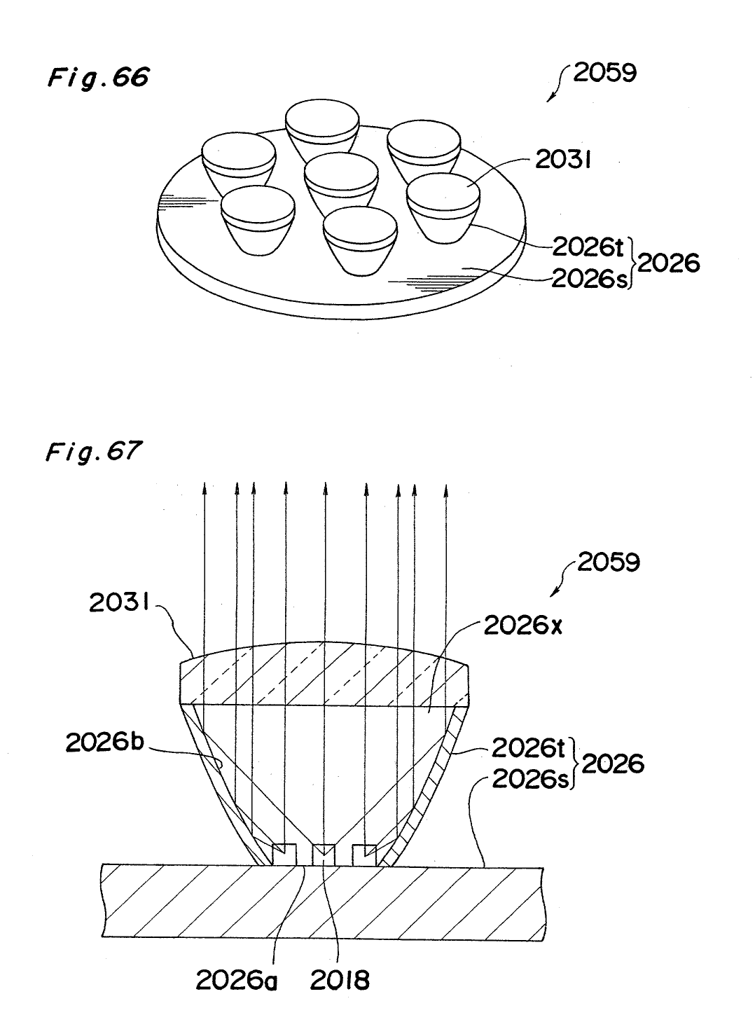

As shown in FIGS. 66 to 75, the illumination device can have a reflecting member.

FIGS. 66 and 67 show an illumination apparatus 2059, as a light emitting module, according to the sixteenth embodiment. As shown in the figure, the illumination apparatus has a supporting member 2026. The supporting member 2026 is formed of a substrate 2026s and reflecting members 2026t each of which is in a cup form.

In a reflecting member 2026t, a through hole 2026x is formed as a recess so that the inner surface 2026b of the through hole 2026x is used as a reflecting surface. The reflecting surface is prepared by finishing the inner surface 2026 as a mirror surface, or by forming a reflecting film thereon by plating or deposition.

The reflecting members 2026t and the substrate 2026s may be connected after being formed separately or may be integrally formed at the same time. In the case that they are formed separately, even if a through hole 2026x is of a complex form, its processing is easy. The cross section (inner surface 2026b) of the reflecting member 2026t is formed in an ellipse or in a parabola so that light is allowed to travel in the forward direction, in a parallel manner, in a condensing manner, or in a spreading manner, or light.

The surface 2026a of the substrate 2026s is exposed from one end of the opening of the through hole 2026s of the reflecting member 2026t inside which one, or two, or more, light emitting elements 2018 are fixed.

A lens 2031 is placed at the other of the opening of the through hole 2026x of the reflecting member 2026t. In accordance with the construction, cooperating with the inner surface 2026b, the light condensing characteristics by the lens 2021 is enhanced.

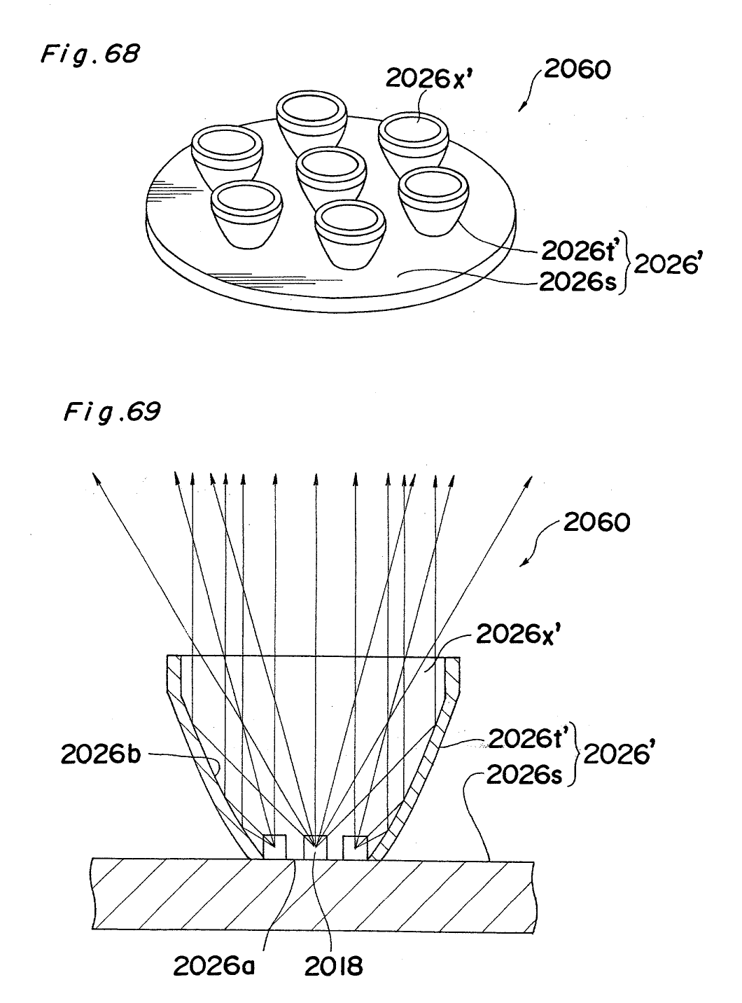

FIGS. 68 and 69 show an illumination apparatus 2060, as a light emitting module, according to the seventeenth embodiment. As shown in the figure, in the construction, there is not provided a lens.

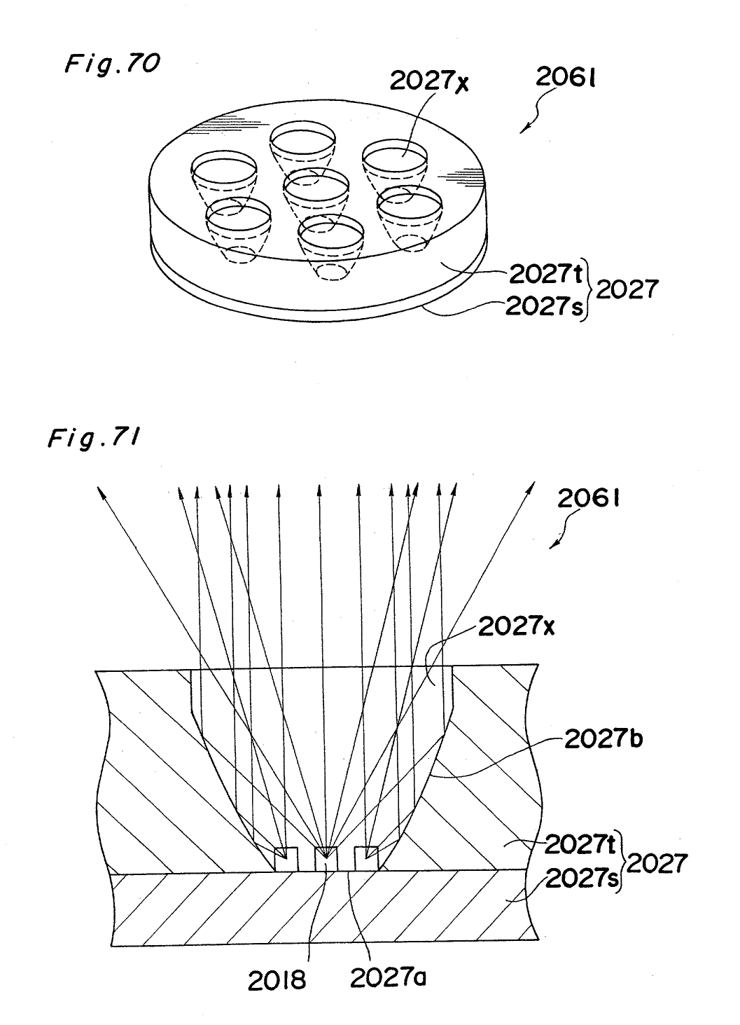

FIGS. 70 and 71 show an illumination apparatus 2061, as a light emitting module, according to the eighteenth embodiment. As shown in the figure, the illumination apparatus has a supporting member 2027. The supporting member 2027 is formed of a substrate 2027s and a reflecting member 2027t. In the construction, one reflecting plate member 2027t wherein a plurality of through holes 2027x is formed, is connected to the substrate 2027s. The illumination apparatus 2061 has a reduced number of components so that the configuration can be simplified. In addition, unevenness due to the formation of reflecting member is eliminated (or omitted) so that its handling also becomes easy.

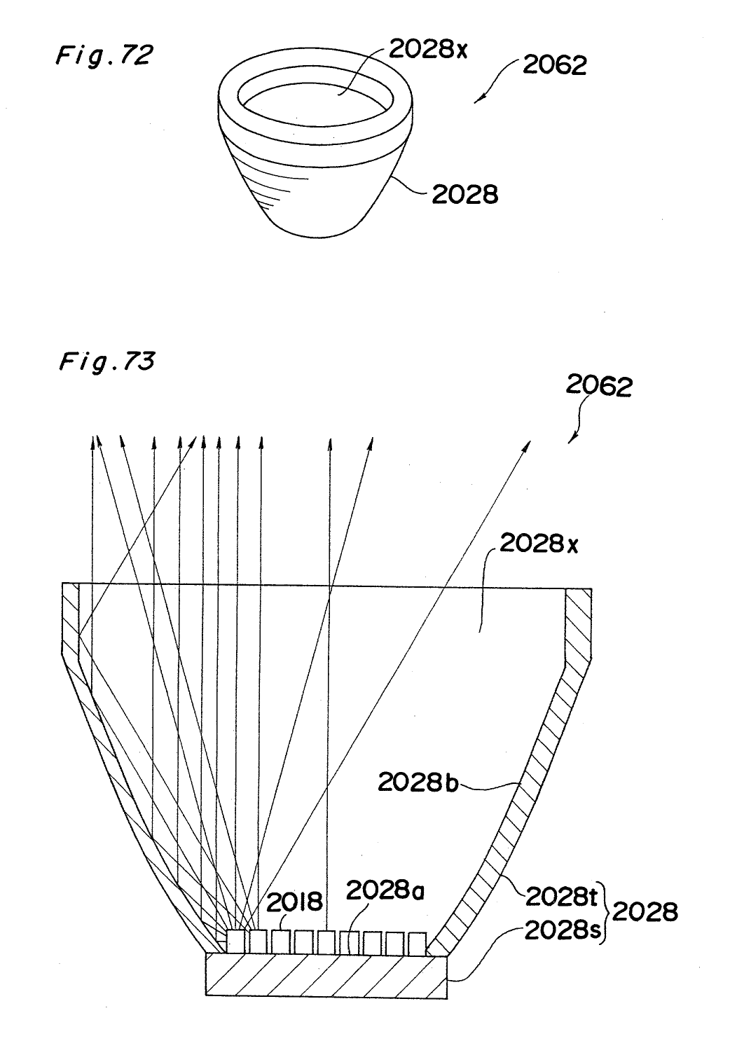

FIGS. 72 and 73 show an illumination apparatus 2062, as a light emitting module, according to the nineteenth embodiment. As shown in the figure, the illumination apparatus has a supporting member 2028. The supporting member 2028 is formed of a substrate 2028s and a reflecting member 2028t. In the construction, the one reflecting plate member 2028t wherein one through hole 2028x is formed, is connected to the substrate 2028s. A plurality of light emitting elements 2018 are arranged on the surface 2028a of the substrate 2028s, that is to say, on the bottom surface of the recess.

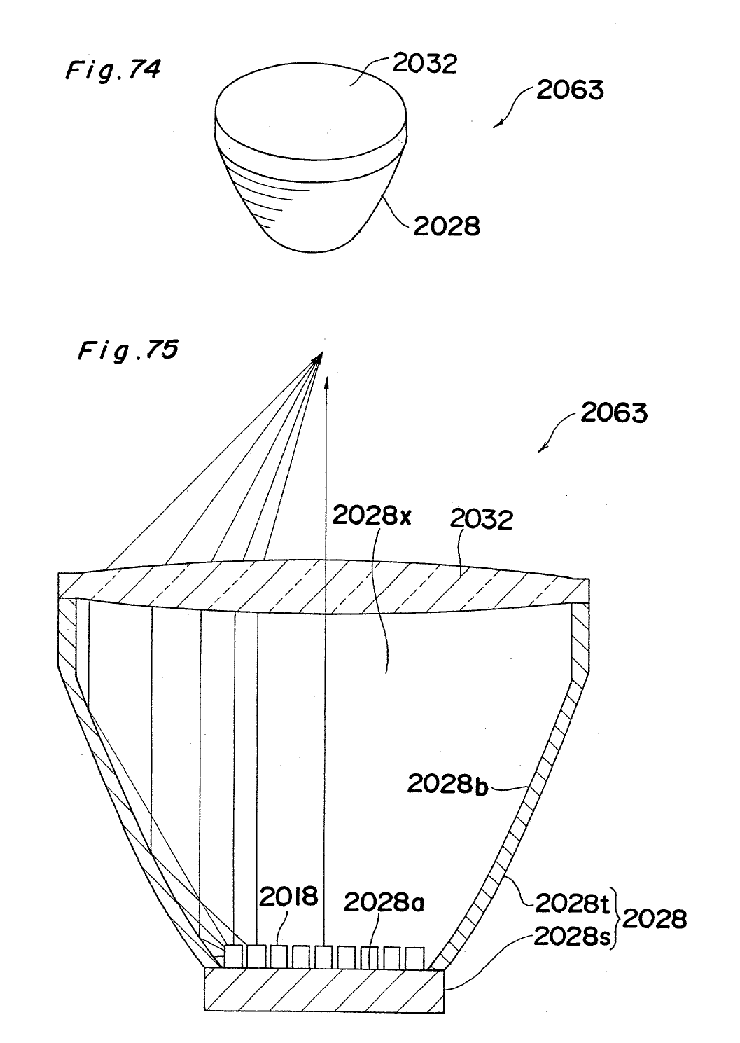

FIGS. 74 and 75 show an illumination apparatus 2063, as a light emitting module, according to the twentieth embodiment. As shown in the figure, the illumination apparatus has a supporting member 2028. The supporting member 2028 is formed of a substrate 2028s and a reflecting member 2028t. In the construction, there is further provided a lens 2032 at one end of an opening of the reflecting plate member 2028t in which one through hole 2028x is formed. Thereby, the condensing characteristics can be enhanced, as shown by arrows in FIG. 75.

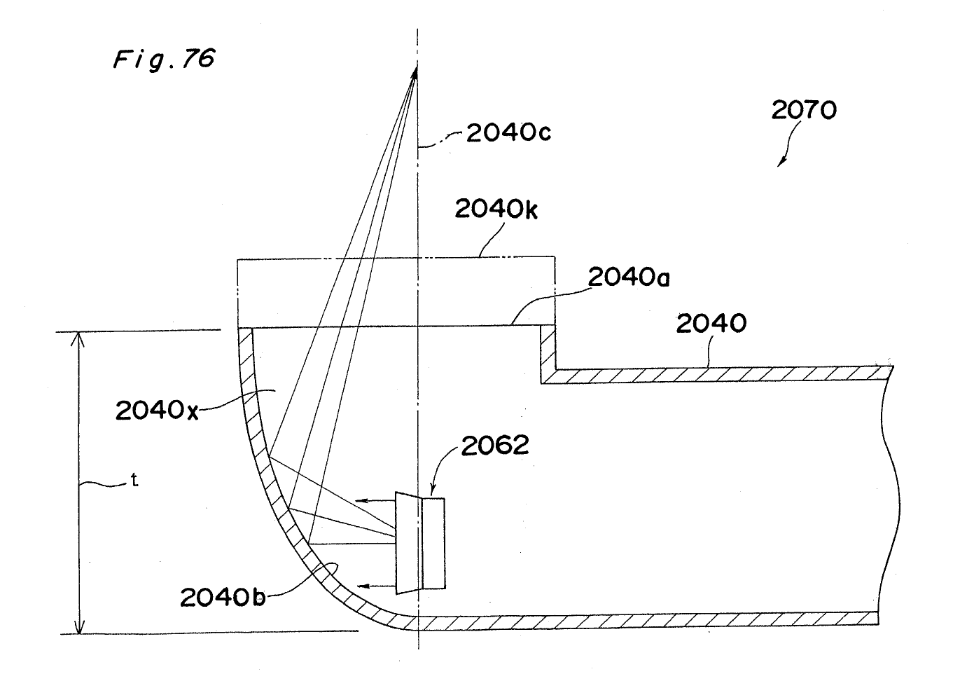

Next, with reference to FIGS. 76 through 92, it is explained about a photopolymerizers for medical use, preferably for dental use, provided with the illumination apparatus, according to each of the embodiments.

The light emitting elements used in the illumination apparatus emit light (for example, blue light) having a wavelength suitable for curing a photopolymerizing resin material (for example, dental resin) of 350 nm to 500 nm, for example, and preferably of 430 nm to 480 nm. The illumination apparatus in which different types of light emitting elements that emit light having differing wavelengths are combined is used, or a plural number of illumination apparatuses of which the light emitting elements emit light having differing wavelengths are used in combination, for a photopolymerizing resin material in which a plural number of materials cured by differing wavelengths are combined so that the respective materials are cured by light having differing wavelengths emitted by the corresponding light emitting elements.

FIGS. 76 to 81 show configuration views of major portions of photopolymerizers 2070 to 2075 for medical use that emit light from illumination apparatuses after reflecting the light from reflecting surfaces formed therein, so as to change the direction of the light. The portions on the tip sides of extension parts 2040 to 2045 that extend from grip parts for gripping, are illustrated. Openings 2040a to 2045b are formed at the tip portions, or in the vicinity thereof, of the extension parts 2040 to 2045, and the light emitting modules, as illumination apparatuses, are arranged in the spaces 2040x to 2045x that are connected to the openings 2040a to 2045a so that light reflected on the reflecting surfaces 2040b to 2045b is emitted to the outside. These photopolymerizers 2070 to 2075 for medical use, have the same appearances as, or similar appearances to, turbines for dental use, in which the openings 2040a to 2045a are formed in the portions corresponding to the tool attachment parts of the heads of the turbines for dental use. In the construction, light is emitted in the direction that forms an angle with respect to the direction in which the extension parts 2040 to 2045 extend.

The photopolymerizer 2070 for medical use shown in FIG. 76, has the opening 2040a in the vicinity of the tip of the extension part 2040, and the space 2040x is connected to the opening 2040a. The reflecting surface 2040b is formed inside of the tip side of the extension part 2040. The illumination apparatus (for example, illumination apparatus 2062 shown in FIGS. 72 and 73) provided with only one reflecting member, is arranged within the space 2040x so as to be opposed to the reflecting surface 2040b.

The reflecting surface 2040b is a portion of a concave surface of an elliptical body of revolution having the axis 2040c as a center, of which the cross section is a portion of an ellipse. The reflecting surface 2040b is formed by, for example, finishing, or polishing, the surface of the material as a mirror surface. or, a dielectric film and a metal film, such as of aluminum, gold, or silver, may be formed on the surface of the material. An optical coating may be applied to the reflecting surface 2040b in order to enhance, or promote, the reflectance thereof.

The illumination apparatus 2062 is placed at one of the focal points, or in the vicinity thereof, of the ellipse of the reflecting surface 2040b so as to emit light toward the reflecting surface 2040b. Light from the illumination apparatus 2062 is reflected by the reflecting surface 2040b as shown by the arrow in the figure and is emitted from the opening 2040a in the vicinity of the tip of the extension part 2040 so as to be collected to the other focal point, or to the vicinity thereof, of the ellipse of the reflecting surface 2040b.

In the case that a lens 2040k is provided at the opening 2040a, it is possible to adjust the convergence (or collection) of light such as by shifting the condensing position of light for illumination or by converting light into parallel light.

The illumination apparatus 2062 may be arranged so as to be shifted from the axis 2040c. Also, instead of forming the reflecting surface 2040b as a surface of an ellipse of revolution, the reflecting surface may be formed simply so that a cross section of the reflecting surface at an arbitrary position in the direction perpendicular to the paper surface in the figure becomes a portion of an ellipse.

In such a configuration, the thickness “t” can be made thin and, therefore, this can be easily inserted into an oral cavity.

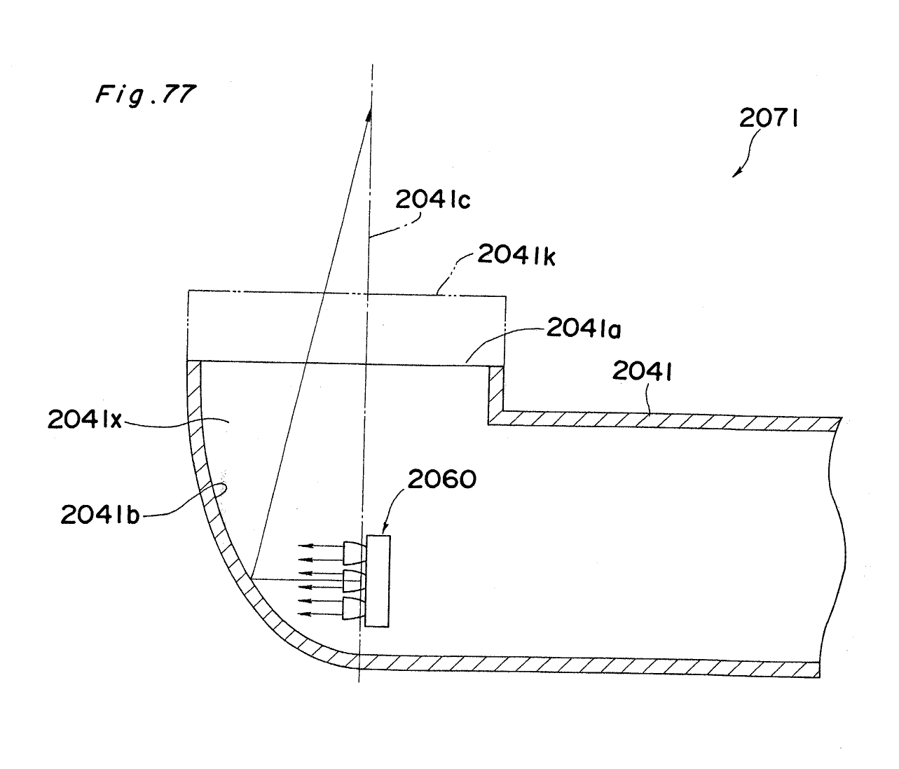

Another type of illumination apparatus may be utilized. For example, an illumination apparatus (for example, illumination apparatus 2059 shown in FIGS. 66 and 67) having a plurality of reflecting members may be used for the photopolymerizer 2071 for medical use shown in FIG. 77. Also, a conventional LED may be used. The configuration of the extension part 2041, the reflecting surface 2041b, and the like, of the photopolymerizer 2071 for medical use are the same as in the photopolymerizer 2070 for medical use of FIG. 76.

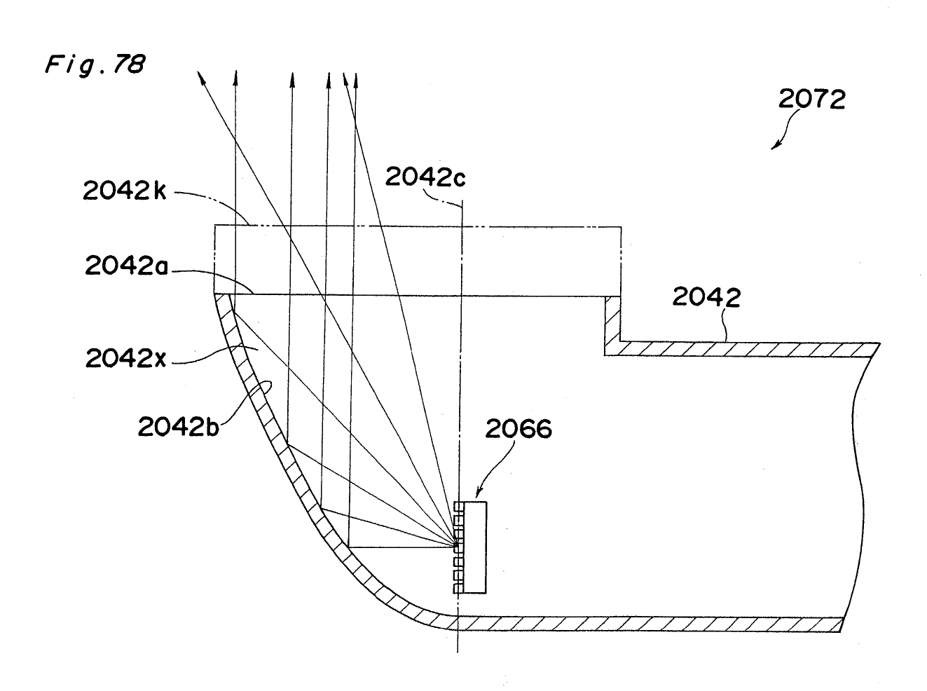

The photopolymerizer 2072 for medical use shown in FIG. 78 employs an integrated wafer 2066 and a reflecting surface 2042b having a cross section in a parabolic form.

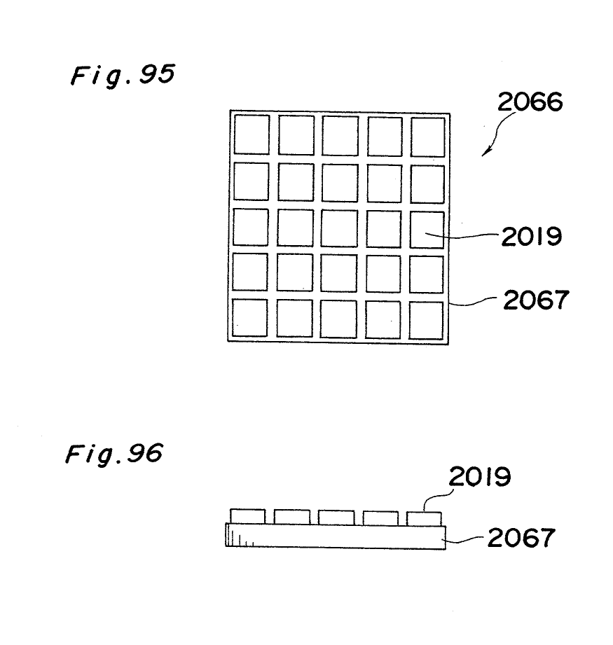

The integrated wafer 2066 has, as shown in FIGS. 95 and 96, a plurality of bare chips 2019 arranged on a substrate 2067 and is a light emitting element that is referred to as, for example, a power LED. As shown in FIG. 78, the integrated wafer 2066 is arranged on the parabolic center axis 2042c of the reflecting surface 2042b so as to emit light in the direction perpendicular to the parabolic center axis 2042c of the reflecting surface 2042b, but it may be arranged in a position shifted from the parabolic center axis 2042c. Also, the light emitting element may have a configuration that is arranged within a recess or may be an LED that is not arranged within a recess.

The reflecting surface 2042b is a portion of the surface of a parabola of revolution having the axis 2042c as the center and has a cross section that is a portion of a parabola. The reflecting surface 2042b is formed, for example, by finishing or polishing the surface of the material as a mirror surface. Or a dielectric film and a metal film such as of aluminum, gold or silver may be formed on the surface of the material. An optical coating for enhancing the reflectance may be applied to the reflecting surface 2040b.

Since the reflecting surface 2042b is a surface of a parabola of revolution, a major portion of light from the integrated wafer 2066 is emitted as parallel light parallel to the parabolic center axis 2042c after being reflected by the reflecting surface 2042b. Light may be condensed by providing a lens 2042k at the opening 2042a.

Here, instead of forming the reflecting surface 2042b as a surface of a parabola of revolution, the reflecting surface may be formed simply so that a cross section of the reflecting surface at an arbitrary position in the direction perpendicular to the paper surface in the figure becomes a portion of a parabola.

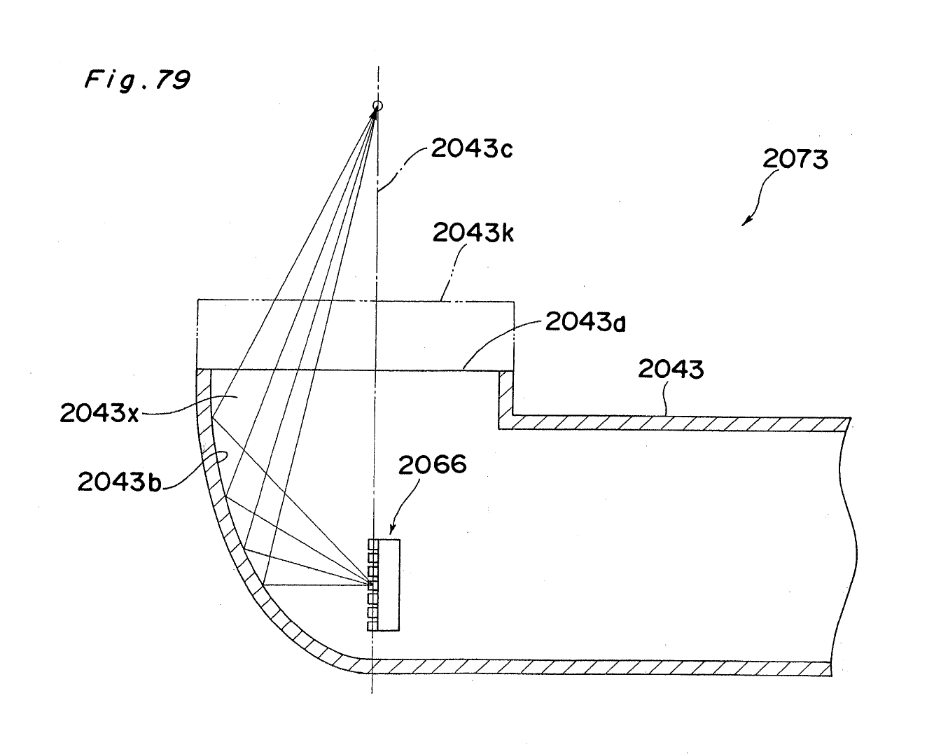

The photopolymerizer 2073 for medical use shown in FIG. 79 is provided with an integrated wafer 2066 and a reflecting surface 2043b having a cross section in an elliptic form.

As shown in the figure, the integrated wafer 2066 is placed at one of the centers, or in the vicinity thereof, of the ellipse of the reflecting surface 2043b so that light reflected from the reflecting surface 2043b is corrected to the other center of the ellipse, or to the vicinity thereof.

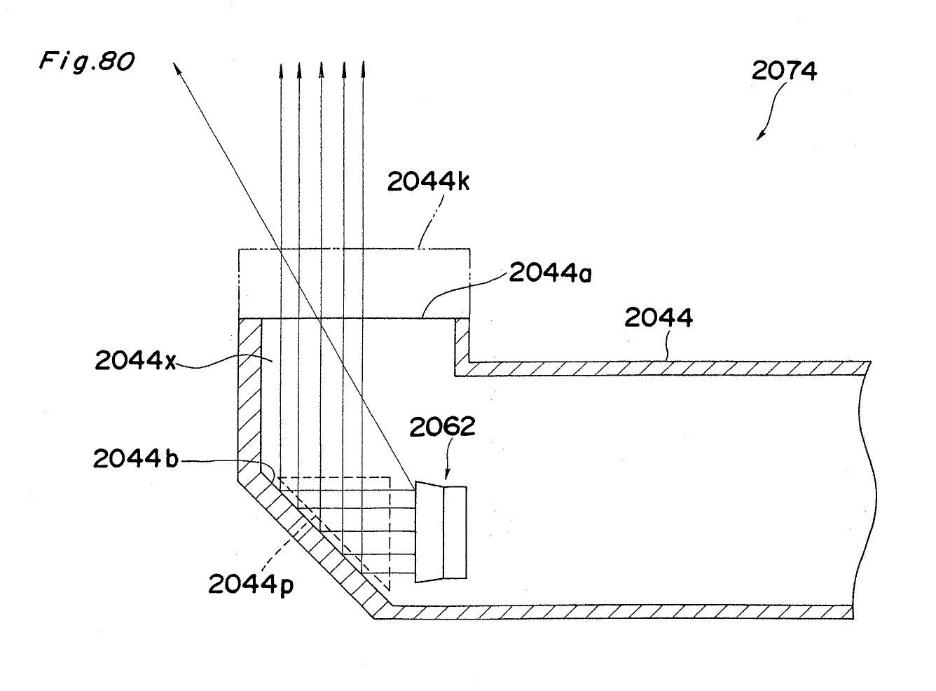

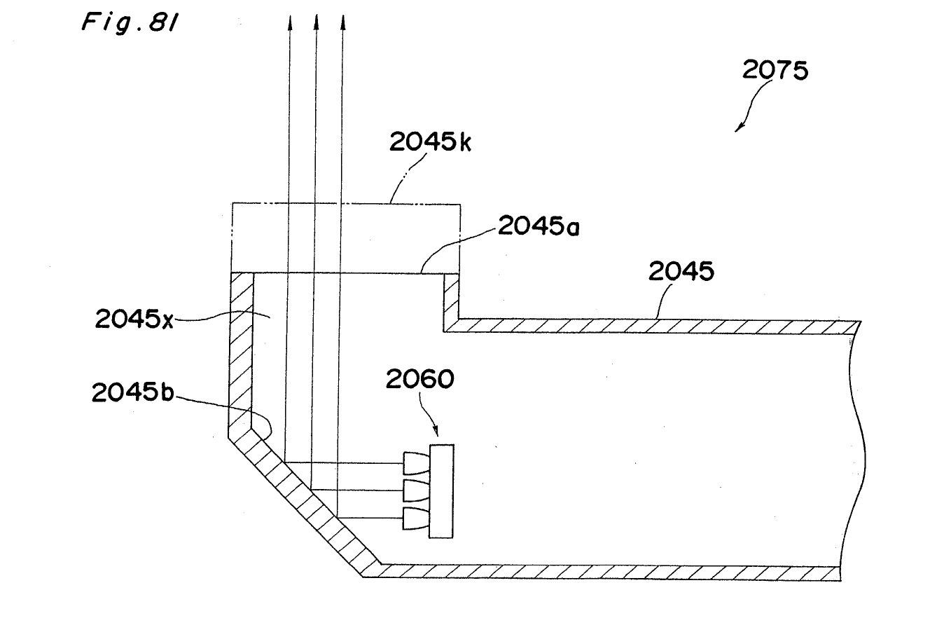

In the photopolymerizer 2074 for medical use shown in FIG. 80, a plane reflecting surface (or flat reflecting surface) 2044b is used, and an illumination apparatus (for example, illumination apparatus 2062 of FIG. 72) having one reflecting member is placed within the extension part 2044. Since the reflecting surface 2044b is plane or flat, parallel light from the illumination apparatus 2062 is changed in the direction from the reflecting surface 2044b and is outputted through the opening 2044a as parallel light without being condensed, as shown by the arrows in the figure. In the case that a lens 2044k is provided at the opening 2044a, light can be condensed.

The reflecting surface 2044b is arranged so as to form an angle of no less than 45 degrees and no greater than 135 degrees with respect to the direction in which the extension part 2044 extends and with respect to the opposite side to the tip portion. Thereby, light is emitted from the opening 2044a in the direction perpendicular to the direction in which the extension part 2044 extends or in the direction tilted towards the user's side (to the grip part side) and, therefore, it becomes easy to handle the photopolymerizer for medical use.

The flat reflecting surface may be formed by using a prism. For example, a equilateral triangular prism 2044p is arranged within the extension part 2044, as shown by the broken lines in FIG. 80.

In the photopolymerizer 2075 for medical use shown in FIG. 81, a plain (or flat) reflecting surface 2045b is used, and an illumination apparatus (for example, illumination apparatus 2060 of FIG. 66) having a plurality of reflecting members is arranged within an extension part 2045.

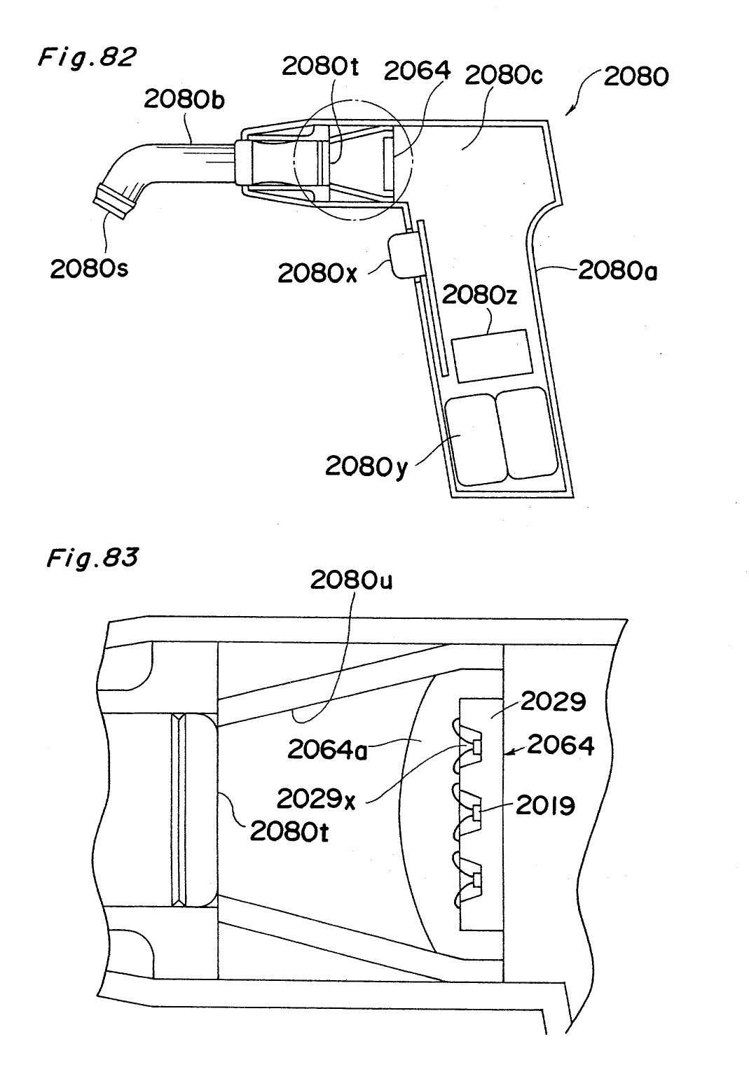

FIGS. 82 to 86 show the configurations of, so-called, gun-type and cordless-type photopolymerizers for medical use.

In a photopolymerizer 2080 for medical use shown in FIG. 82, a light guide 2080b is attached to an end portion of a housing 2080a formed in approximately an L-shape. An illumination apparatus 2064 is arranged within the housing 2080a so as to be opposed to one end surface 2080t of the light guide 2080b. With the construction, the light is emitted from the other end surface 2080s of the light guide 2080b. The photopolymerizer 2080 for medical use is of a cordless-type as aforementioned, in which an electric power source battery 2080y and a control circuit substrate 2080z are accommodated within the housing 2080a. In the construction, when an operational switch 2080x protruding from the housing 2080a is pressed, the light emitting elements 2019 in the illumination apparatus 2064 emit light, which is reflected on a reflecting surface 2080u of the reflecting member and is then condensed so as to enter the end surface 2080t of incidence of the light guide 2080b. Then, the light is emitted from a free end surface 2080s for irradiation of light. An operator grips the housing 2080a for utilization. The light emitting elements 2019 are provided inside of the extension part 2080c that extends from the grip part. Though it is preferable for the light emitting elements to be arranged within recesses, the photopolymerizer for medical use may have a configuration without recesses.

In an illumination apparatus 2064, as shown in FIG. 83 which is an enlarged view corresponding to a chained line in FIG. 82, the light emitting elements 2019 of bare chips are arranged in recesses 2029x formed in a substrate 2029, in the same manner as in the illumination apparatus 2053 shown in FIGS. 52 to 54. The illumination apparatus 2064 may be provided with a lens 2064a in order to enhance the light condensing characteristics in order to efficiently utilize the light.

The light guide 2080b having a high possibility of coming into contact with the teeth can be removed from the photopolymerizer 2080 for medical use so as to be sterilized. In addition, a variety of light guide 2080b having differing curved forms and/or sizes can be prepared, and any particular one can be selected and mounted on the photopolymerizer for medical use according to a particular purpose of utilization.

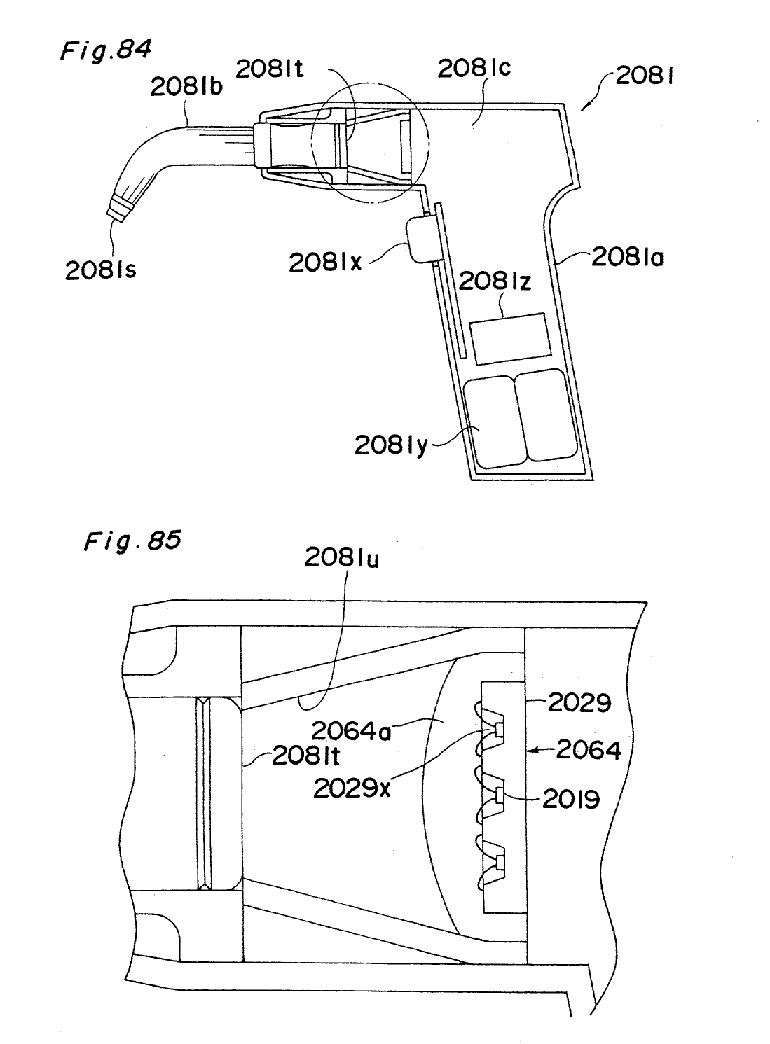

In a photopolymerizer 2081 for medical use shown in FIGS. 84 and 85, the outgoing light side (or side of irradiation of light) of the light guide 2081b is narrowed towards its tip side. Namely, the end surface 2081s on the outgoing light side (or the tip side) is made smaller so as to enhance the degree of condensing light. The other parts are formed in the same manner as that of the photopolymerizer 2080 for medical use in FIGS. 82 and 83. The housing 2081a is grasped by hand for utilization. The light emitting elements 2019 are provided inside the extension part 2081c that is extended from the grip part. Light from the illumination apparatus 2064 is reflected on the reflecting surface 2081u of the reflecting member, and the collected light is allowed to enter an end surface 2081t, corresponding to incidence of light, of the light guide 2081b so as to be emitted from the end surface 2081s, corresponding to irradiation of light.

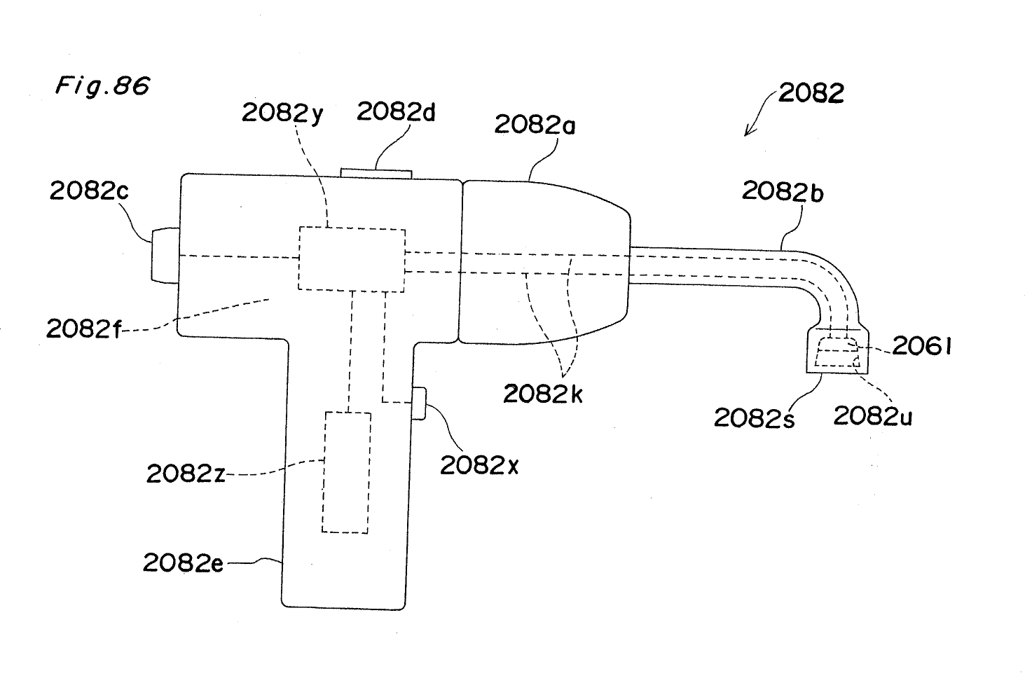

A photopolymerizer 2082 for medical use, shown in FIG. 86, is provided with, for example, the illumination apparatus 2061, as the light emitting module, of FIGS. 70 and 71 at the tip of the extension part 2082b which is connected to a housing 2082a so that the light is emitted from the tip 2082s of the extension part 2028b. Alternatively, the illumination apparatus 2061 may be provided inside of the extension part 2082f that extends from the grip part 2082e so that light is emitted from the tip 2082s of the extension part 2028b via a light guide. The light emitted from the illumination apparatus 2061 is emitted toward its front after being reflected on a reflecting surface 2082u of a reflecting member mounted at the tip of the extension part 2082b.

An output adjustment part 2082c, a display part 2082d and an operational switch 2082x are arranged on the surface of the housing 2082a, and a control substrate (i.e. control board) 2082y and an electric power supply battery 2082z are arranged inside of the housing. The electric power is supplied to the illumination apparatus 2061 from the control substrate 2082y via a lead wire 2082k. Here, each of the gun-type photopolymerizers, shown in FIGS. 82 to 86, is not limited to such a cordless type. Namely, each thereof can be formed as a type having an electric cord.

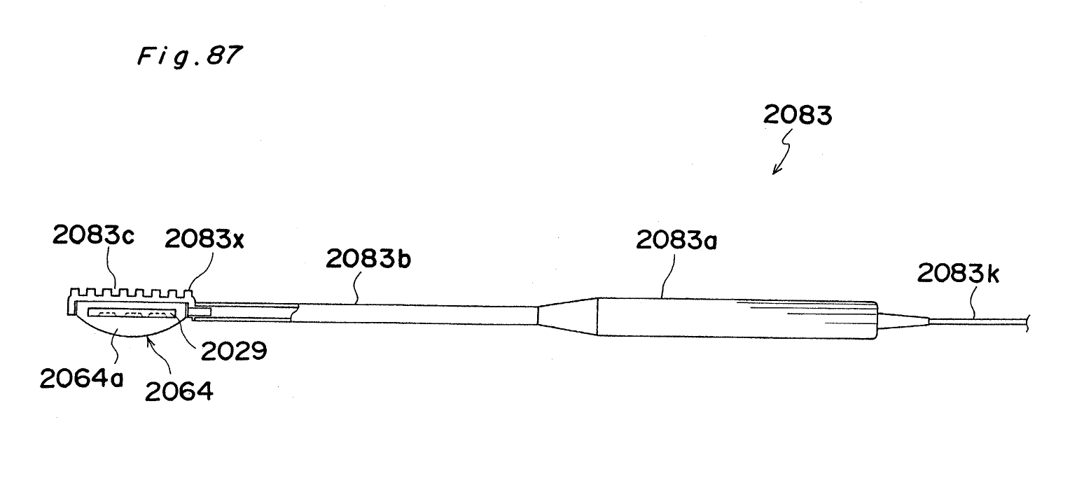

Each of photopolymerizers shown in FIGS. 87 to 92 is constructed as a so-called dental mirror-type photopolymerizer for medical use, in which the form thereof is similar to that of a dental mirror.

In the photopolymerizer 2083 for medical use shown in FIG. 87, the light emitting module 2064 is arranged at the tip portion 2083c of an elongate extension part 2083b connected to a grip part 2083a which is gripped by the hand of a user. The substrate 2029 side of the illumination apparatus 2064 is attached to the tip portion 2083 of the elongate extension part 2082b so that light is emitted in a direction different from, for example in the direction perpendicular to, the direction in which the elongate extension part 2083b extends. An electric power supply cord 2083k for supplying the electric power to the illumination apparatus 2064 is connected to the grip part 2083a.

Cooling fins 2083x are provided at the tip portion 2083c of the extension part 2083b. The cooling fins 2083x is formed in a cylindrical concentric form by using, for example, a metal material so that heat generated from the illumination apparatus 2064 is transmitted to the cooling fins 2083x, and so that the heat is dissipated, or discharged, from the cooling fins 2083x.

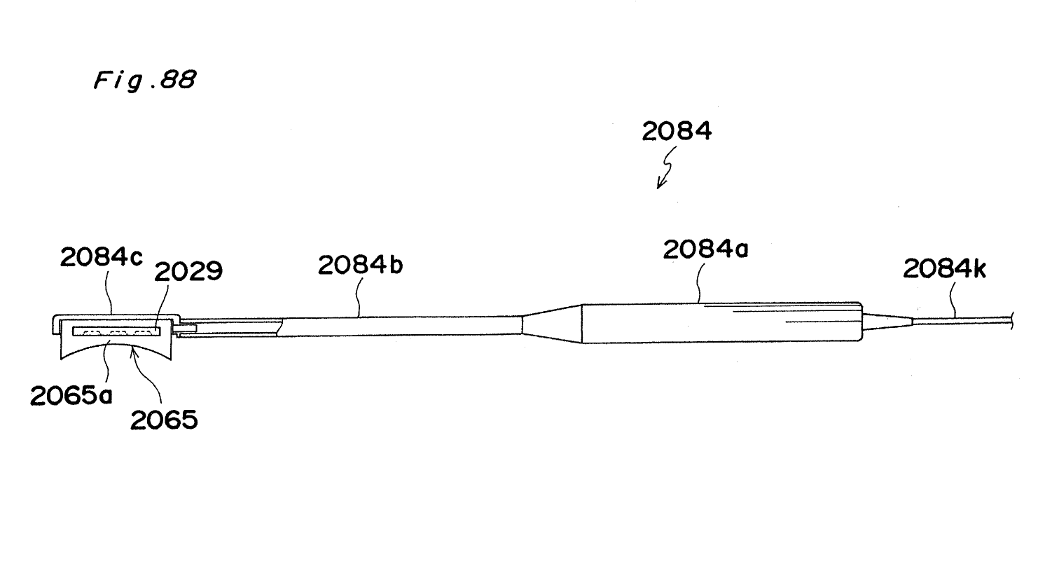

Alternatively, like the mirror-type photopolymerizer 2084 for medical use as shown in FIG. 88, the photopolymerizer can be constructed so that it has no cooling fins. Alternatively, the form of the lens 2065a provided with the light emitting module 2065, as the illumination apparatus, may be changed so as to have different condensing characteristics. The other parts of the configuration of the photopolymerizer 2084 for medical use are the same as in the photopolymerizer 2083 for medical use shown in FIG. 87.

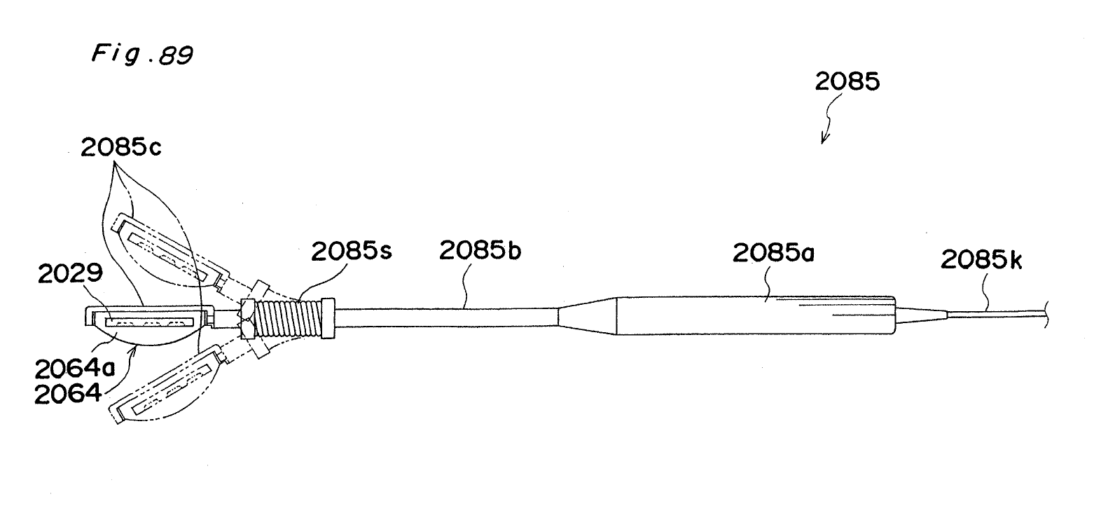

In a photopolymerizer 2085 for medical use shown in FIG. 89, a flexible part 2085s is provided in a middle part of the extension part 2085b. The flexible part 2085s has a flexibility to the degree that it can be bent by hand as shown by the chained lines in the figure and that the bent condition can be maintained. The flexible part 2085s can be appropriately bent and, thereby, the light can be emitted in a desired direction while the grip part 2085a is being gripped at a desirable angle at which the grip part 2085a is easily grasped. The other parts of the configuration of the photopolymerizer 2085 for medical use are the same as those in the photopolymerizer 2083 for medical use shown in FIG. 87.

Here, instead of providing the flexible part 2085s as a portion of the extension part 2085b, the entirety of the extension part 2085b may be formed so as to be bendable as a flexible part.

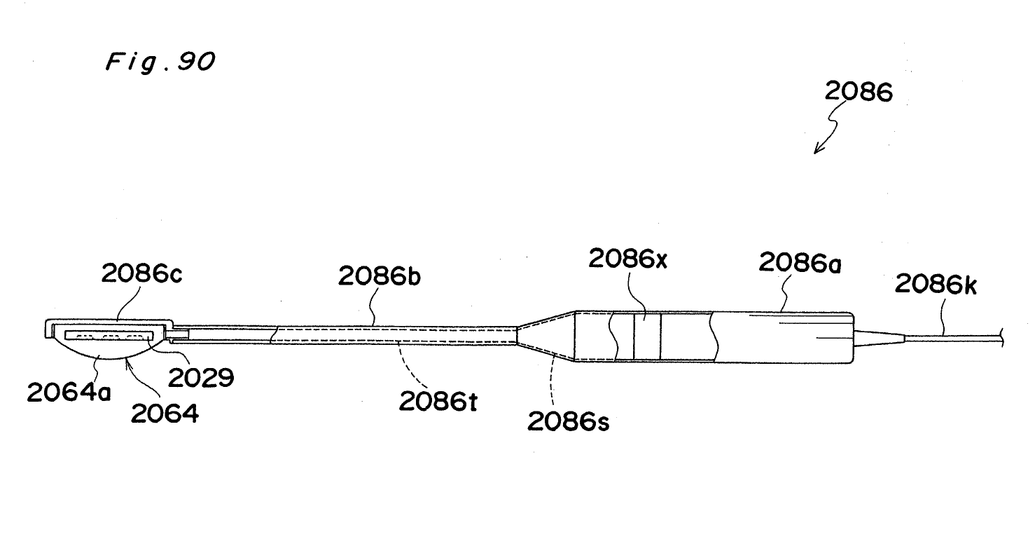

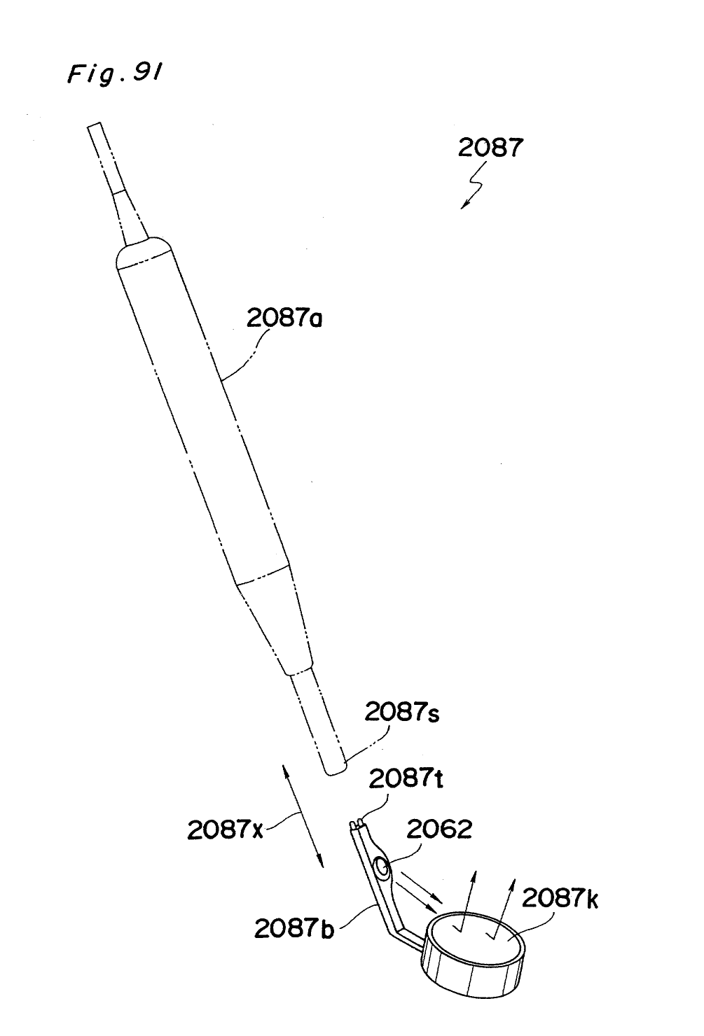

A photopolymerizer 2086 for medical use, shown in FIG. 90, is provided with a fan 2086x within the grip part 2086a so as to cool the light emitting module 2064 as the illumination apparatus. The extension part 2086b and the grip part 2086a are formed to be hollow so that air paths 2086s and 2086t are arranged through the hollow area. In the construction, the air for cooling is sent from the fan 2086x to the illumination apparatus 2064 that is attached to the tip portion 2086c of the extension part 2086b. The electric power is supplied to the fan 2086x from the electric power supply cord 2086k.

FIG. 91 shows a photopolymerizer 2087 for medical use which is constructed as a type having a mirror with a light. When a tip portion 2087b is connected to a body 2087a of the photopolymerizer 2087, connectors 2087s and 2087t are electrically connected to each other so that the light emitting module, as the illumination apparatus (for example, the illumination apparatus 2062 shown in FIGS. 72 and 73), provided at the tip portion 2087b emits blue light suitable for curing a photopolymerizing resin material toward a mirror 2087k positioning at the tip portion 2087b.

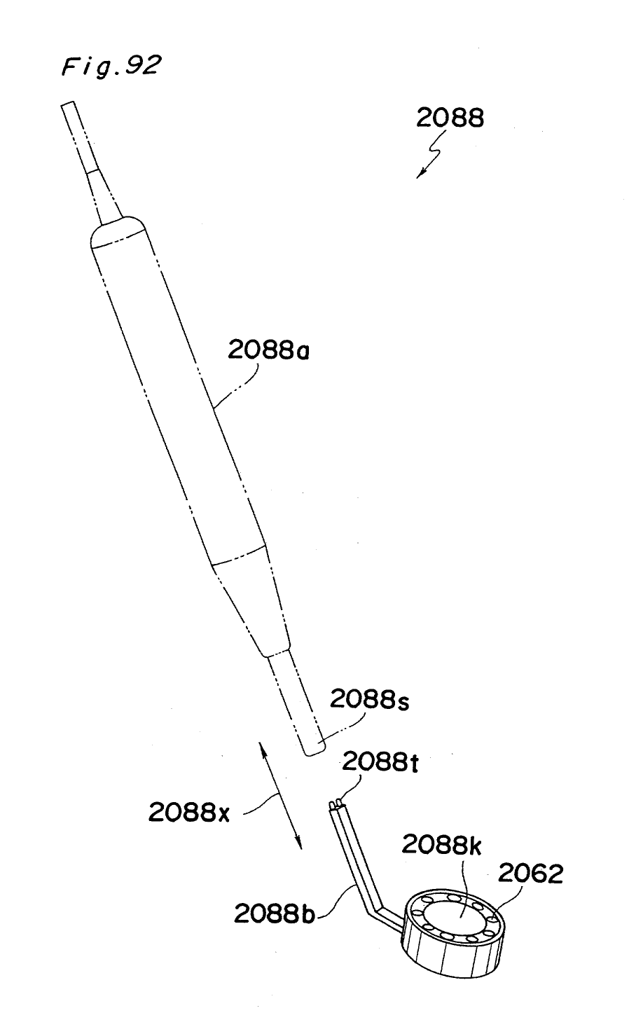

FIG. 92 shows a case in which a photopolymerizer 2088 for medical use is employed as a photopolymerizer. When a tip portion 2088b is connected to a body 2088a of the photopolymerizer, connectors 2088s and 2088t are electrically connected to each other so that the light emitting module as the illumination apparatus (for example, the illumination apparatus 2062 shown in FIGS. 72 and 73) arranged around a mirror 2088k at the tip portion 2088b emits blue light suitable for curing a photopolymerizing resin material.

As described above, in the photopolymerizer for medical use according to each of the above described embodiments, light from the light emitting elements can be effectively utilized by reflecting the light from the light emitting elements and, thereby, miniaturization and enhancement of output power can be achieved.

The present invention is not limited to each of the above described embodiments, and the present invention can be implemented in a variety of embodiments and modifications other than the above.

For example, the present invention can apply not only to the dental field, but also to the medical field at large. Also, the medical applications are not limited to a direct treatment or diagnosis, but they can also be directed towards preparation and/or formation of objects like dentures by dental technicians employing such photopolymerizers.

Also, not only the LED but also, for example, semiconductor laser, organic El, or the like, may be used as the light emitting element.

Also, it is possible to combine the above described illumination apparatus with the above described photopolymerizer in a variety of manners, in addition to, for example, the combinations shown in the above described embodiments.

What is claimed is:

1. A medical illuminator, comprising:
a base member; and
a plurality of light emitting elements for emitting light, in which the light emitting elements are integrated and provided in the base member, and in which the base member and the plurality thereof are formed as a light emitting module.

2. The medical illuminator as claimed in claim 1, wherein each of the plurality of light emitting elements is one of a bare chip and a chip element.

3. The medical illuminator as claimed in claim 2, wherein the light emitting module comprises a light collector which has one of a shape and a construction for collecting the light emitted from the light emitting elements.

4. The medical illuminator as claimed in claim 3, wherein the light collector comprises one of a lens for converging the light emitted from the light emitting elements in which the lens is provided on a side on which the light is emitted from the light emitting elements and a light converter for making parallel the light emitted from the light emitting elements in which the light converter is provided on the side.

5. The medical illuminator as claimed in claim 2, wherein the light emitting module is flat in shape, and
wherein the light is output from a main surface of the light emitting module.

6. The medical illuminator as claimed in claim 2, wherein the light emitting module is covered by a transparent resin on at least a side on which the light emitting elements emit the light.

7. The medical illuminator as claimed in claim 6, wherein the light emitting module is sealed by the transparent resin.

8. The medical illuminator as claimed in claim 2, which further comprises a cooler for cooling the light emitting module.

9. The medical illuminator as claimed in claim 2, wherein each of the light emitting elements is one of a light emitting diode and a laser semiconductor.

10. A medical light irradiator for photocuring which comprises the medical illuminator as claimed in claim 2, wherein the light emitted from the light emitting module is employed for curing photocuring resin material.

11. The medical light irradiator as claimed in claim 10, wherein the light emitting elements emit lights with different wavelengths.

12. The medical light irradiator as claimed in claim 11, wherein the light emitting elements include at least one first element for emitting white light and include at least one second element for emitting blue light, and
wherein the white light and the blue light can be selectively irradiated.

13. The medical light irradiator as claimed in claim 10, wherein there is provided a light collector inside the light emitting module.

14. The medical light irradiator as claimed in claim 13, wherein the light emitting module has a shape which has a property of collecting the light.

15. The medical light irradiator as claimed in claim 14, wherein each of the light emitting elements is provided with a predetermined angle in the light emitting module so that a light emitting surface of the each thereof is orientated towards a common point.

16. The medical light irradiator as claimed in claim 13, wherein the light collector comprises one of a lens for converging the light emitted from the light emitting elements in which the lens is provided on a side on which the light is emitted from the light emitting elements and a light converter for making parallel the light emitted from the light emitting elements in which the light converter is provided on the side.

17. The medical light irradiator as claimed in claim 10, wherein the light emitting module is flat in shape, and
wherein the light is output from a main surface of the light emitting module.

18. The medical light irradiator as claimed in claim 10, wherein the light emitting elements are driven by pulse.

19. The medical light irradiator as claimed in claim 10, wherein the light emitting module is provided on a tip part of the medical light irradiator.

20. The medical light irradiator as claimed in claim 19, which further comprises:
an elongate supporter; and
a light outputting part for outputting the light emitted from the light emitting module, in which one end of the elongate supporter is connected to the light outputting part,
wherein a direction in which the light is outputted from the light outputting part is different from a direction in which the elongate supporter extends.

21. The medical light irradiator as claimed in claim 19, which further comprises:
an elongate supporter; and
a light outputting part for outputting the light emitted from the light emitting module, in which one end of the elongate supporter is connected to the light outputting part,
wherein the elongate supporter has a flexible part which can be bent into a desirable shape and maintain the desirable shape.

22. The medical light irradiator as claimed in claim 10, which further comprises a cooler for cooling the light emitting module.

23. The medical light irradiator as claimed in claim 22, wherein the cooler has one of a fan, a Peltier element, and a heatsink.

24. The medical light irradiator as claimed in claim 22, which further comprises:
an elongate supporter; and
a light outputting part for outputting the light emitted from the light emitting module, in which one end of the elongate supporter is connected to the light outputting is part,
wherein the cooler is a ventilator through which a cooling air for cooling the light emitting module passes.

25. The medical light irradiator as claimed in claim 22, in which the cooler is a fan for cooling the light emitting module.

26. The medical light irradiator as claimed in claim 25, wherein the light emitting module and the fan for cooling the light emitting module are provided on a tip part of the medical light irradiator.

27. The medical light irradiator as claimed in claim 22, wherein the cooler is a heatsink which is provided on the light emitting module.

28. The medical light irradiator as claimed in claim 27, which further comprises a fan for cooling the heatsink.

29. The medical light irradiator as claimed in claim 10, which further comprises a metal housing inside which the light emitting module is installed.

30. The medical light irradiator as claimed in claim 10, wherein one of a light guide and an outer lens is provided in opposition to the light emitting module.

31. The medical light irradiator as claimed in claim 30, wherein the light guide is a taper type light guide.

32. The medical light irradiator as claimed in claim 30, wherein the one of the light guide and the outer lens is connected to the light emitting module detachably.

33. The medical light irradiator as claimed in claim 32, wherein the light guide can be selected from a plurality of light guides with different shapes.

34. The medical light irradiator as claimed in claim 10, wherein there are provided a controller for controlling emission of the light from the light emitting elements and a battery for supplying electricity to both of the controller and the light emitting elements, in a housing of the medical light irradiator.

35. A medical instrument which comprises the medical illuminator as claimed in claim 2, wherein the light emitted from the light emitting module is employed for illuminating oral cavity.

36. The medical instrument as claimed in claim 35, wherein each of the light emitting elements is a light emitting diode which emits white light.

37. The medical instrument as claimed in claim 35, wherein the light emitting elements include at least one first element for emitting white light and include at least one second element for emitting blue light, wherein the white light and the blue light can be selectively irradiated.

38. The medical instrument as claimed in claim 35, wherein the light emitting module is arranged at one of a location corresponding to a head of the medical instrument and a location in vicinity of the head.

39. The medical instrument as claimed in claim 35, which further comprises a light guide for leading the light from the light emitting module to a light projecting part which is provided at one of a location corresponding to a head of the medical instrument and a location in vicinity of the head.

40. The medical instrument as claimed in claim 35, wherein an air is employed for cooling the light emitting module.

41. A medical unit which comprises the medical illuminator as claimed in claim 2, wherein the light emitted from the light emitting module is employed for illumination.

42. The medical unit as claimed in claim 41, wherein the light emitting elements include at least one first element for emitting white light and include at least one second element for emitting blue light, wherein the white light and the blue light can be selectively illuminated.

43. A medical light irradiator for photocuring which comprises the medical illuminator as claimed in claim 1.

44. The medical light irradiator as claimed in claim 43, which further comprises:
a light leading part which has a surface of incidence and a surface of irradiation that is smaller than the surface of incidence, in which the light emitted from the light emitting module, enters the surface of incidence, is led to the surface of irradiation, and is irradiated from the surface of irradiation,
wherein the light emitted from the light emitting module is a light suitable for curing photocuring resin material.

45. The medical light irradiator as claimed in claim 43, which further comprises:
a light leading part which has a surface of incidence and a surface of irradiation, in which the light emitted from the light emitting module, enters the surface of incidence, is led to the surface of irradiation, and is irradiated from the surface of irradiation,
wherein the light emitted from the light emitting module is a light suitable for curing photocuring resin material, and
wherein the light emitting module and the light leading part are provided on an end of the medical light irradiator.

46. The medical light irradiator as claimed in claim 45, wherein the surface of irradiation is smaller than the surface of incidence in area.

47. The medical light irradiator as claimed in claim 45, wherein the light leading part is detachably provided on a housing of the medical light irradiator.

48. The medical light irradiator as claimed in claim 47, wherein the light leading part can be selected from a plurality of light leading parts with different shapes.

49. The medical light irradiator as claimed in claim 43, which further comprises one of a conversion lens for narrowing directivity of the light emitted from the light emitting module, and a condenser for condensing the light emitted from the light emitting module and for directly irradiating the light toward outside,
wherein the light emitted from the light emitting module is a light suitable for curing photocuring resin material, and
wherein the light emitting module, and the one of the conversion lens and the condenser, are provided on an end of the medical light irradiator.

50. The medical light irradiator as claimed in claim 49, which further comprises a converting lens for narrowing directivity of the light emitted from the light emitting elements, wherein the converting lens is provided between the light emitting elements and the condenser.

51. The medical light irradiator as claimed in claim 43, wherein the light emitting module is supported by a tip portion of an elongate supporter, and
wherein a direction in which the light is emitted from the light emitting module, is different from a direction in which the elongate supporter extends.

52. The medical light irradiator as claimed in claim 51, wherein the light emitting module is flat in shape, and
wherein the light is output from a main surface of the light emitting module.

53. The medical light irradiator as claimed in claim 43, which further comprises an elongate supporter having a tip portion,
wherein a tip portion member is connected to the tip portion, and
wherein the light emitting module is provided inside the tip portion member.

54. The medical light irradiator as claimed in claim 43, which further comprises an elongate supporter, in which the light emitting module is supported by an end part of the elongate supporter,
wherein the elongate supporter has a flexible part which can be bent into a desirable shape and maintain the desirable shape.

55. The medical light irradiator as claimed in claim 43, wherein each of the light emitting elements is provided with a predetermined angle in the light emitting module so that the light emitted from the light emitting elements is irradiated towards a common point,
wherein there is provided a light leading part which has a surface of incidence and a surface of irradiation that is smaller than the surface of incidence, in which the light emitted from the light emitting module, enters the surface of incidence, is led to the surface of irradiation, and is irradiated from the surface of irradiation, and
wherein the surface of incidence is located at the common point.

56. The medical light irradiator as claimed in claim 43, wherein the plurality of light emitting elements include light emitting elements which emit lights having different wavelengths.

57. The medical light irradiator as claimed in claim 43, wherein the light emitting elements are driven by pulse.

58. The medical light irradiator as claimed in claim 43, wherein there are provided a controller for controlling emission of the light from the light emitting elements and a battery for supplying electricity to both of the controller and the light emitting elements, in a housing of the medical light irradiator.

59. The medical light irradiator as claimed in claim 43, wherein the light is a light suitable for curing photocuring resin, and
wherein there is provided a cooler for cooling the plurality of light emitting elements.

60. The medical light irradiator as claimed in claim 59, wherein the cooler is built in the light emitting module.

61. The medical light irradiator as claimed in claim 43, which further comprises:
a reflection surface for reflecting the light emitted from each of the light emitting elements,
wherein the light emitted from the light emitting module is a light suitable for curing photocuring resin material.

62. The medical light irradiator as claimed in claim 61, wherein the base member comprises a support member having one or more concave parts in which the light emitting elements are provided, and
wherein the support member has a plurality of reflecting surfaces in the concave parts, in which the reflecting surfaces are part of the reflection surface, and in which the light emitted from the light emitting elements is reflected by the reflecting surfaces so that the light reflected thereby is led toward openings of the concave parts.

63. The medical light irradiator as claimed in claim 62, wherein each of the reflecting surfaces in the concave parts has a cross-sectional shape which includes at least a part of one of an ellipse and a parabola.

64. The medical light irradiator as claimed in claim 62, wherein each of the light emitting elements is a bear chip, and
wherein the support member is a substrate, in which each of the reflecting surfaces forms on at least a part of each of the concave parts.

65. The medical light irradiator as claimed in claim 64, which further comprises an optical element which has one of a function for collecting the light irradiated from the openings of the concave parts and a function for making parallel the light irradiated from the openings thereof.

66. The medical light irradiator as claimed in claim 65, wherein the optical element is one of a lens having a spherical surface and a lens having a non-spherical surface.

67. The medical light irradiator as claimed in claim 66, wherein the lens is mounted on each of the openings of the concave parts, and
wherein each of the concave parts is filled up by a transparent resin.

68. The medical light irradiator as claimed in claim 64, wherein the substrate is made of one of ceramic, alumina, and metal coated with insulating material.

69. The medical light irradiator as claimed in claim 64, wherein each of the light emitting elements is positioned away from a bottom surface of the each of the concave parts formed in the substrate.

70. The medical light irradiator as claimed in claim 64, wherein the bear chip is fixed on the substrate at each of the concave parts, by wireless bonding.

71. The medical light irradiator as claimed in claim 64, wherein the bear chip is made of an integrated wafer.

72. The medical light irradiator as claimed in claim 64, wherein each of the reflecting surfaces is formed on at least a part of each of the concave parts of the substrate, in which the each of the reflecting surfaces has a cross-sectional shape which includes at least a part of one of an ellipse and a parabola.

73. The medical light irradiator as claimed in claim 64, wherein each of the reflecting surfaces is a reflective coating formed on the substrate corresponding to each of the concave parts.

74. The medical light irradiator as claimed in claim 61, wherein each of the light emitting elements is a bear chip,
wherein the base member comprises a support member which has:
a substrate on which the light emitting elements are provided; and
a reflecting member which has a penetration hole an inner surface of which surrounds the light emitting elements arranged on the substrate, in which the reflecting member is provided on the substrate, and
wherein a reflecting surface is formed on at least a part of an inner surface of the reflecting member, in which the reflecting surface is part of the reflection surface.

75. The medical light irradiator as claimed in claim 74, which further comprises an optical element which has one of a function for collecting the light irradiated from the penetration hole of the reflecting member and a function for making parallel the light irradiated from the penetration hole thereof.

76. The medical light irradiator as claimed in claim 75, wherein the optical element is one of a lens having a spherical surface and a lens having a non-spherical surface.

77. The medical light irradiator as claimed in claim 76, wherein the lens is mounted on an opening of the penetration hole of the reflecting member, and
wherein the penetration hole is filled up by a transparent resin.

78. The medical light irradiator as claimed in claim 74, wherein the substrate is made of one of ceramic, alumina, and metal coated with insulating material.

79. The medical light irradiator as claimed in claim 74, wherein the light emitting elements are positioned away from the substrate.

80. The medical light irradiator as claimed in claim 74, wherein the bear chip is fixed on the substrate, by wireless bonding.

81. The medical light irradiator as claimed in claim 74, wherein the bear chip is made of an integrated wafer.

82. The medical light irradiator as claimed in claim 74, wherein the reflecting surface being formed on at least the part of the inner surface of the reflecting member has a cross-sectional shape which includes at least a part of one of an ellipse and a parabola.

83. The medical light irradiator as claimed in claim 74, wherein the reflecting surface is a reflective coating formed on the inner surface of the penetration hole of the reflecting member.

84. The medical light irradiator as claimed in claim 61, which further comprises:
a holding part which is held by hand; and
an extension part which extends from the holding part,
wherein one of a tip part of the extension part and a part near the tip part, has an opening, in which the light emitting elements are provided in a space connecting to the opening.

85. The medical light irradiator as claimed in claim 84, wherein one of a light which is collected and a parallel light, is irradiated from the opening.

86. The medical light irradiator as claimed in claim 85, wherein the light emitting elements emit the light generally in a direction in which the extension part extends, and
wherein the reflection surface is provided inside the space, in which the reflection surface reflects the light emitted from the light emitting elements in a direction different from the direction in which the extension part extends.

87. The medical light irradiator as claimed in claim 86, wherein the reflection surface has a cross-sectional shape which includes at least a part of one of an ellipse and a parabola.

88. The medical light irradiator as claimed in claim 86, wherein the reflection surface is a flat surface, and
wherein an angle formed by the flat surface with respect to the direction in which the extension part extends and with respect to the holding part, is between 45 degrees and 135 degrees.

89. The medical light irradiator as claimed in claim 61, which further comprises a light guide,
wherein the light emitting elements are provided in opposition to an edge surface of incidence of the light guide.

90. The medical light irradiator as claimed in claim 89, wherein one of a light which is collected and a parallel light, is irradiated from the light emitting elements toward the edge surface of incidence of the light guide.

91. The medical light irradiator as claimed in claim 61, which further comprises a cooler for cooling the light emitting elements.
